(19) United States
(12) Patent Application Publication
(10) Pub. No.: US 2024/0335537 A1
(43) Pub. Date: Oct. 10, 2024

(54) ENGINEERED SOLUBLE DECOY RECEPTORS TO ENHANCE CANCER IMMUNOTHERAPY

(71) Applicant: Baylor College of Medicine, Houston, TX (US)

(72) Inventors: Pradip Bajgain, Houston, TX (US); Juan Fernando Vera Valdes, Houston, TX (US); Ann Marie Leen, Houston, TX (US); Mohsen Basiri, Houston, TX (US)

(73) Assignee: Baylor College of Medicine, Houston, TX (US)

(21) Appl. No.: 18/681,931

(22) PCT Filed: Aug. 12, 2022
(86) PCT No.: PCT/US2022/074917

§ 371 Date: Feb. 7, 2024

Related U.S. Application Data
(60) Provisional application No. 63/232,352, filed on Aug. 12, 2021.

(51) Int. Cl. A61K 39/00; A61K 38/00; A61P 35/00; C07K 14/54; C07K 14/55; C07K 14/705; C07K 14/71; C07K 14/715

(52) U.S. Cl. CPC A61K 39/4635; A61K 39/4611; A61K 39/4613; A61P 35/00; C07K 14/5418; C07K 14/5443; C07K 14/55; C07K 14/70578; C07K 14/71; C07K 14/7155; A61K 38/00

(57) ABSTRACT

Disclosed herein are methods and compositions for modulating immune responses by using decoy molecules to bind to soluble or other ligands. In specific embodiments, the decoy molecules comprise a domain that binds to a target immunosuppressive ligand and another domain that releases immunostimulatory signals when activated. In specific embodiments, the decoy molecules are soluble and are secreted by transgenic T cells at a tumor site, such as upon antigen engagement, and they protect the transgenic cells themselves and also bystander (e.g., non-modified) endogenous immune cells.

This application claims priority to U.S. Provisional Patent Application Ser. No. 63/232,352, filed Aug. 12, 2021, which is incorporated by reference herein in its entirety.

### TECHNICAL FIELD

Embodiments of the disclosure encompass at least the fields of cell biology, molecular biology, and medicine.

### BACKGROUND

The use of chimeric antigen receptor-modified T cells has been associated with remarkable clinical success when used to treat hematologic malignancies. However, extension to solid tumors has proven more challenging, likely due to a number of factors including the hostile tumor microenvironment (TME) replete with suppressive cells and molecules designed to limit effector T cell function. One such example is FasL, a molecule expressed by a range of TME cells including tumor cells, tumor endothelium, polymorphonuclear myeloid derived suppressor cells (PMN-MDSCs), M2 macrophages, regulatory T cells (Tregs), and cancer-associated fibroblasts (CAFs), which, upon engagement with its endogenous receptor Fas (expressed by activated T cells), induces apoptotic T cell death.

Under normal physiological conditions, Fas/FasL signaling plays a crucial role in maintaining immune cell homeostasis. For example, contraction of antigen-specific cytotoxic T lymphocytes following resolution of an immune response is partially mediated by FasL-induced apoptosis. Similarly, Fas/FasL signaling plays an important role in preventing potential autoimmune reactivity due to continued T cell activation and proliferation even after eradication of the threat. However, tumors exploit this pathway as a mechanism to evade immune-mediated elimination. Indeed, in preclinical studies, Zhu et al. demonstrated that FasL-expressing PMN-MDSCs found in TiRP melanoma tumors were able to induce apoptotic cell death of tumor-infiltrating lymphocytes (TILs)—an effect that was alleviated by blocking Fas/FasL engagement. Similarly, Lakins and colleagues showed that CAFs and other stromal cells recruited to the tumor site in mice engrafted with B16.OVA tumors could induce OVA-specific CD8+ T cell death via Fas. Finally, elevated levels of FasL in both tumor and serum samples have been detected clinically in various solid tumors including bladder, breast, cervical, gastric, pancreatic cancers, and sarcoma, and correlated with disease progression, increased metastasis, and poor patient survival. For instance, Zietz and colleagues reported that FasL was detected in >70% of the 40 angiosarcoma tumors analyzed, and found that high level expression correlated with decreased CD8+ TILs and inferior patient survival.

The present disclosure provides solutions to needs in the art of cancer therapy, including at least by overcoming hindrances from the TME.

### BRIEF SUMMARY

The present disclosure is directed to methods and compositions related to cancer therapy. In particular embodiments, the disclosure provides methods and compositions for treatment of cancer of any kind, including solid tumors or hematological malignancies.

Embodiments of the disclosure encompass soluble recombinant proteins comprising (1) at least one inhibitory protein domain, wherein the inhibitory protein domain can bind to at least one immunosuppressive ligand, and (2) at least one activating protein domain. In some cases, the inhibitory protein domain comprises an extracellular domain selected from the group consisting of TGFBR2, FAS, IL4R, IL10R, and a combination thereof. In some cases, the immunosuppressive ligand is selected from the group consisting of TGF-β, FASL, IL4, IL10, or a combination thereof. The inhibitory protein domain may comprise one or more mutations from a natural protein sequence of the inhibitory protein domain. In specific cases, the activating protein domain comprises at least one domain selected from the group consisting of IL-2, IL-7, IL-15, and a combination thereof. At least one inhibitory protein domain may be linked to at least one activating protein domain via a protein linker. An example of a protein linker comprises a G-S linker, such as the G-S linker comprises the protein sequence comprising GGGSGGGGSGGGGSGGG (SEQ ID NO:1).

In specific embodiments, the recombinant protein comprises: an extracellular domain of TGFBR2 and IL-2; an extracellular domain of TGFBR2 and IL-7; an extracellular domain of TGFBR2 and IL-15; an extracellular domain of FAS and IL-2; an extracellular domain of FAS and IL-7; an extracellular domain of FAS and IL-15; an extracellular domain of IL4R and IL-7; an extracellular domain of IL4R and IL-15; an extracellular domain of IL4R and IL-2; an extracellular domain of IL10R and IL-2; an extracellular domain of IL10R and IL-7; or an extracellular domain of IL1OR and IL-15.

Also included are nucleic acids comprising a sequence encoding any recombinant protein encompassed herein. In specific embodiments, there are one or more vectors comprising a nucleic acid sequence encoding any recombinant protein encompassed herein. Specific examples include viral vectors or non-viral vectors. A genetic vector may comprise a transient expression vector or a stable expression vector. Cells may comprise any recombinant protein encompassed herein and/or any vector encompassed herein. Examples of cells include the following: T lymphocyte, a natural killer cell, a macrophage, a mesenchymal stromal cell, tumor infiltrating cell, NK cell, NK T cell, fibroblast, or mixture thereof. The cell may or may not be genetically modified to express at least one additional recombinant protein, such as one or more engineered antigen receptors and/or antibodies of any kind. In specific cases, the engineered receptor comprises one or more chimeric antigen receptors and/or one or more transgenic T cell receptors. In specific examples, the cell is a T cell, NK cell, or NK T cell comprising one or more chimeric antigen receptors and/or one or more transgenic T cell receptors. In specific cases, the cell is a tumor-specific T cell generated by ex vivo antigen/peptide stimulation.

In specific embodiments, a cell comprises a recombinant protein comprising 1, 2, or more of the following: an extracellular domain of TGFBR2 and IL-2; an extracellular domain of TGFBR2 and IL-7; an extracellular domain of TGFBR2 and IL-15; an extracellular domain of FAS and IL-2; an extracellular domain of FAS and IL-7; an extracellular domain of FAS and IL-15; an extracellular domain of IL4R and IL-2; an extracellular domain of IL4R and IL-7; an extracellular domain of IL4R and IL-15; an extracellular domain of IL10R and IL-2; an extracellular domain of IL10R and IL-7; or an extracellular domain of IL10R and IL-15.

Embodiments of the disclosure include methods of treating an individual comprising administering a therapeutically effective amount of any recombinant protein encompassed herein and/or a therapeutically effective amount of any cells encompassed herein. In specific embodiments, the method comprises administering a therapeutically effective amount of any cells encompassed herein and comprises administering a therapeutically effective amount of cells comprising anti-cancer activity. In specific embodiments, the cells are autologous with respect to the individual and are further defined as immune cells engineered to express the protein. In some embodiments, the cells are allogeneic with respect to the individual and are further defined as immune cells engineered to express the protein. In some methods, the immune cells are T lymphocyte, a natural killer cell, a macrophage, a mesenchymal stromal cell, tumor infiltrating cell, NK cell, or NK T cells. In some cases of the method, the cell are autologous or allogeneic with respect to the individual and are further defined as virus-specific T cells. In specific embodiments, the cells comprising anti-cancer activity comprise one or more engineered antigen receptors or one or more antibodies, any of which target a cancer antigen. In particular embodiments of the method, the individual has or is suspected of having cancer. Any therapeutically effective amount may comprise a single dose or multiple doses. In some cases of the method, the method further comprises administering a therapeutically effective amount of one or more additional therapeutic compositions to the individual, such as one or more compositions that can activate immune responses directly or indirectly. The method may further comprise the step of selecting the recombinant protein based on a cancer type of the individual.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1G show CAR T cells are susceptible to tumor-expressed FasL mediated apoptosis. (1A) Representative and summary FACS data demonstrating CAR transduction efficiency, day 5 post transduction (mean±SEM, n=5). Statistical significance was calculated using unpaired t test, p<0.01. (1B) Cytolytic activity of non-transduced control T cells (NT) and CAR.PSCA T cells (CAR) tested in ⁵¹Cr-release assay against PSCA-ve 293T and PSCA+ve CAPAN1 and CFPAC1 pancreatic cancer cells (mean±SEM, n=5). Statistical difference was calculated using unpaired t test, p<0.05. (1C) Fas (CD95) expression on a representative donor T cells (1D) T cell Fas expression summary data (mean±SEM, n=5) Significance determined using unpaired t test, p<0.0001. (1E) Viability of CAR T cells exposed to various concentrations of recombinant FasL (1F) Representative FACS plots showing expression of FasL by CAPAN1 and CFPAC1 tumor cells under normal culture conditions and after 24 hr exposure to IFNγ. (1G) Survival of CAR.PSCA T cells 3 days after exposure to FasL+ CAPAN1 tumor cells in absence or presence of Fas-blocking antibody ZB4 (mean±SEM, n=4). (1H) Illustration demonstrating the susceptibility of CAR T cells to suppression by solid tumors via the Fas/FasL signaling axis.

FIGS. 2A-2H show developing an engineered Fas decoy receptor to sequester FasL. (2A) Schematic diagram of Fas decoy function. (2B) Fas decoy (FD) construct schema (top) and representative FACS plot demonstrating CAR and CAR+FD transduction efficiency, day 5 post transduction (bottom). (2C) Summary FACS data indicating expression of the CAR and CAR+decoy transgenes by T cells (mean±SEM, n=4). (2D) Phenotype analysis of CAR.PSCA and CAR. FD T cells assessed 10 days post-transduction (mean±SEM, n=3) (2E) Comparison of cell expansion of CAR and CAR.FD T cells. (2F) Secretion of effector cytokines by CAR and CAR.FD T cells. (2G) Comparison of cytolytic function of CAR and CAR.FD T cells in chromium release assay. (2H) Secretion of fas decoy (FD.0) molecule by CAR-only and CAR.FD T cells in absence or presence of recombinant PSCA 48-hours post stimulation as measured by soluble Fas ELISA (mean±SEM, n=4). Statistical difference was calculated using unpaired t test, p<0.05;

FIGS. 3A-3D show Fas decoy-engineered T cells exhibit superior function in presence of FasL. (3A) Neutralization of recombinant FasL by FD.0 molecules present in conditioned media obtained from FD-expressing 293T cells (293T.FD) tested in an ELISA based assay. (3B) Summary FACS data demonstrating viability of CAR.PSCA T cells when cultured overnight in conditioned media obtained from activation cultures of 293T or 293T.FD T cells (mean±SEM, n=3). Statistical difference between treatment groups was calculated using unpaired t test. (3C) Quantitative data showing anti-tumor activity of CAR.PSCA and CAR.FD T cells indicated by fold change in tumor cell numbers in co-culture with CAPAN1 tumor cells (mean±SEM, n=4). Statistical difference was determined using one-way ANOVA, p<0.05. (3D) Summary data showing quantification of T cells during the coculture experiment (mean±SEM, n=4). Significance was calculated using unpaired t test, p<0.05;

FIGS. 4A-4G show combining the Fas decoy and IL-15 fusion protein (FD.15) to enhance T cell activity (4A) Schematic diagram of Fas decoy and IL-15 fusion (FD+15) transgene. (4B) Representative FACS plot demonstrating CAR PSCA, CAR.FD, and CAR.FD+15 transduction efficiency, day 5 post transduction. (4C) Summary FACS data indicating expression of the CAR, CAR.FD, and CAR.FD+15 transgenes by T cells (mean±SEM, n=4). Statistical difference was determined using one-way ANOVA, p<0.05. (4D) Secretion of FD+15 molecule by activated T cells measured by Fas and IL-15 ELISAs. (4E) Expansion of CAR, CAR.FD, and CAR.FD+15 T cells upon stimulation. (4F) Anti-tumor activity of CAR, CAR.FD, and CAR.FD+15 T cells in co-culture against CAPAN1 tumor cells measured by co-culture assay. (4G) Quantification of T cells by flow cytometry in the co-culture experiment illustrated in panel F.

FIGS. 5A-5G show decoy engineered CAR T cells demonstrate enhanced in vivo anti-tumor activity (5A) Schematic representation of the in vivo model for the assessment of the decoy engineered T cells. (5B) Summary of the weight of the mice in the course of the in vivo study (mean±SEM, n=5). (5C) Representative mice images demonstrating localization and expansion of the non-transduced (NT) cells by bioluminescence imaging (left), summary of T cell bioluminescence (line) and tumor volume (bars) on the right panel. (5D) Representative mice images demonstrating localization and expansion of the CAR PSCA cells by bioluminescence imaging (left), summary of T cell bioluminescence (line) and tumor volume (bars) on the right panel. (5E) Representative mice images demonstrating localization and expansion of the CAR.FD cells by bioluminescence imaging (left), summary of T cell bioluminescence (line) and tumor volume (bars) on the right panel. (5F) Representative mice images demonstrating localization and expansion of the CAR.FΔ+15 cells by bioluminescence imaging (left), summary of T cell bioluminescence (line) and tumor volume (bars) on the right panel. (5G) Representative mice images demonstrating localization and expansion of the CAR.FD+15 cells by bioluminescence imaging (left), summary of T cell bioluminescence (line) and tumor volume (bars) on the right panel. Statistical differences in tumor volumes between different treatment groups calculated using one-way ANOVA, p<0.05 on day 35;

FIGS. 6A-6E show decoy-engineered T cells promote activity of bystander effector cells (6A) Schematic representation of the in vivo model for the assessment of bystander effects of decoy engineered T cells. (6B) Representative mice images demonstrating localization and expansion of the NT cells by bioluminescence imaging (left), summary of T cell bioluminescence (line) and tumor volume (bars) on the right panel. (6C) Representative mice images demonstrating localization and expansion of the CAR PSCA and CAR.MUC1 T cells by bioluminescence imaging (left), summary of T cell bioluminescence (lines) and tumor volume (bars) on the right panel. (6D) Representative mice images demonstrating localization and expansion of the CAR.FD and CAR.MUC1 T cells by bioluminescence imaging (left), summary of T cell bioluminescence (lines) and tumor volume (bars) on the right panel. (6E) Representative mice images demonstrating localization and expansion of the CAR.FD+15 and CAR.MUC1 T cells by bioluminescence imaging (left), summary of T cell bioluminescence (line) and tumor volume (bars) on the right panel.

FIG. 7 shows expression of MUC1 antigen by tumor cells on day 35 post T cell administration of T cells;

FIGS. 8A-8C show the suppressive effect of the tumor microenvironment. (8A) A Tumor cell secreting a suppressive signal that is then received by a surface receptor of a nearby T cell and subsequent T cell anergy. (8B) Modified T cell releasing a decoy protein, and subsequent neutralization of tumor-derived suppressive signal. (8C) Modified T cell releasing a decoy protein, that simultaneously neutralizes tumor derived suppressive signal and provides stimulation of surrounding T cells.

FIG. 9 shows a single domain soluble decoy capable of neutralizing targeted immunosuppressive ligands (left panel) and a double domain soluble decoy capable of neutralizing targeted immunosuppressive ligands and providing immunostimulatory signals (right panel).

FIG. 10 shows modularity of the components forming the fusion decoy. Various combinations can be designed depending on the target (suppressive signal) and payload (stimulatory signal).

FIG. 11 shows the concentration of suppressive signal relative to concentration of a decoy.

FIG. 12 shows immune cell number over time in the presence of a fusion decoy, a decoy, or no decoy.

FIG. 13 shows the expected window of therapeutic effect as a function of decoy concentration for two different delivery systems.

FIG. 14 shows an example of a bystander effect when using cells producing low or high amounts of decoy

FIG. 15 shows number of activated by stander cells relative to number of low-decoy producing cells.

FIG. 16 shows number of activated by stander cells relative to number of high-decoy producing cells.

FIG. 17 shows free suppressive signal concentration relative to decoy concentration. The horizontal dotted line represents an arbitrary concentration of the suppressive molecules above which they induce immunosuppression. The vertical dotted lines indicate the doses of decoy needed to neutralize the suppressive signals.

FIG. 18 shows decoy and suppressive signal concentration over time when using cells transiently expressing decoy.

FIG. 19 shows decoy and suppressive signal concentration over time with multiple administrations of transiently decoy expressing cells

FIGS. 20-37 show different embodiments for administration of decoy, decoy producing cells, and/or effector cells to an individual.

FIG. 38 shows decoy derived from engineered effector cells, including CAR T cells, γδ TCRs, Tumor CTLs, or NK cells. Effector cells used in combination with decoy include CAR T cells, γδ TCRs, Tumor CTLs, or NK cells.

FIGS. 39A-39B show examples of manufacturing transient expressing decoy T cells. (39A) T cells are cultured from a patients PBMCs which are then transfected with mRNA. The transfected T cells now produce decoy and can be administered right away while remaining cells are cryopreserved for additional infusions (39B) Decoy concentration from T cells transfected with mRNA encoding the decoy decreases over time.

FIG. 40 shows cells expansion over time when decoy is introduced at the beginning of a culture. The figure also represents expansion in an individual after decoy administration.

FIG. 41 shows cells expansion over time when decoy is introduced at the beginning and during a culture. The figure also represents expansion in an individual after decoy administration.

FIG. 42 shows cells expansion over time when decoy is introduced multiple times during a culture. The figure also represents expansion in an individual after decoy administration.

FIG. 43 shows cells expansion over time when decoy is introduced at the beginning and multiple times during a culture. The figure also represents expansion in an individual after decoy administration.

FIGS. 44-66 show different embodiments of administering one or more decoy therapies optionally with one or more non-decoy therapies.

FIGS. 67A-67E shows generated PSCA-targeting CAR T cell and cancer cell line characteristics. (67A) Schematic representation of the second-generation engineered human codon-optomized CAR containing an anti-PSCA scFv, an IgG2 hinge-CH3 domain, and a CD28 costimulatory endodomain. (67B) PSCA levels in 293T, CAPAN-1, and CFPAC-1 cells. (67C) CD95 levels in T cells over time. (67D) Expression of FasL in various cancer cell lines. (67E) Expression of Fas in various cancer cell lines.

FIGS. 68A-68D shows FD and Fas concentration levels in transgenic cell lines. (68A) FD levels when stimulated with plate-bound recombinant PSCA. (68B) Levels when stimulated with K562-PSCA. (68C) Duration of FD over time. (68D) Stability of FD at 37° C.

FIG. 69 shows Annexin V and 7-AAD staining in a representative donor after 24 hours of exposure to supernatants.

FIGS. 70A-70B. FIG. 70A shows expression of markers in CAR, CAR.FD, and CAR.FD+15 cell lines and FIG. 70B shows effector cytokine production.

FIGS. 71A-71B shows CAR.MUC1 characteristics. (71A) Schematic representation of CAR.MUC1. (71B) MUC1 expression after treatment with CAR T cells described herein.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, “allogeneic” refers to tissues or cells or other material from another body that in a natural setting are immunologically incompatible or capable of being immunologically incompatible, although from one or more individuals of the same species.

The term “autologous” as used herein refers to cells or tissues obtained from the same individual.

The term “decoy” as used herein refers to a molecule, such as a recombinant protein including certain recombinant proteins encompassed herein, that binds to a soluble or a cell-surface molecule in order to block the molecule from one or more activities, such as binding to a protein (or a receptor) on a cell. In some embodiments, the recombinant protein comprises or embodies a decoy. The decoy, by binding to the soluble or cell-surface expressed molecule, stops the molecule from triggering a signal cascade, in specific embodiments. For example, a decoy capable of binding to a soluble TGF-β molecule blocks the TGF-β from binding the TGF-β receptor (TGFBR) expressed on cells and the resultant signaling cascade. The decoy, in some embodiments, activates an immune response. In some embodiments, “decoy” is used interchangeably with “decoy product.” In specific cases, the decoy comprises an inhibitory moiety, such as an extracellular domain of a cellular receptor, fused with a moiety that is capable of modulating the activity of immune cells. The source of the decoy can be cell-generated, in specific embodiments.

As used herein, “decoy therapy”, “decoy therapeutic”, or “decoy treatment” may be used interchangeably and refer to therapeutic compositions comprising at least one decoy. A decoy therapy, decoy therapeutic, and/or decoy treatment may be administered to an individual.

The term “engineered” “or “engineering” as used herein refers to an entity that is generated by the hand of man (or the process of generating same), including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they have reduced expression of one or more endogenous genes and/or because they express one or more heterologous genes (such as a decoy), in which case(s) the engineering is all performed by the hand of man. With respect to an antigen receptor, the antigen receptor may be considered engineered because it comprises multiple components that are genetically recombined to be configured in a manner that is not found in nature, such as in the form of a fusion protein of components not found in nature so configured.

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

The disclosure provides compositions and methods for overcoming biological barriers at the TME, including at least FasL-related biological barriers. Given the detrimental impact of tumor-derived FasL on both endogenous and adoptively transferred immune effectors, the disclosure provides embodiments demonstrating protection of effector T cells using a novel secreted Fas decoy molecule as one example, which in some embodiments is referred to as FD. This example of a molecule was designed, among other benefits, (a) to be produced upon T cell engagement with cognate antigen at the tumor site; and (b) developed to shield both transgenic and bystander (non-modified) cells from FasL-induced cell death. Using pancreatic cancer models, only as one representative type of cancer, embodiments herein demonstrate the enhanced T cell expansion and sustained effector function of decoy-modified T cells in vitro and in vivo. Furthermore, by additionally incorporating the pro-proliferative cytokine IL-15, embodiments herein are able to promote T cell growth and survival selectively at the tumor site, thereby producing potent and durable anti-tumor effects. In specific embodiments, the decoy molecule and cytokine are selectively produced upon antigen engagement in the TME.

### II. Compositions

Aspects of the present disclosure concern recombinant proteins capable of modulating immune responses. The recombinant protein, in certain aspects of the disclosure, binds soluble molecules that inhibit immune function. In certain embodiments, the recombinant protein activates immune function. The recombinant protein may be a soluble protein, and when expressed in a cell, may be transported out of, or secreted from, the cell. The release from the cells may be active or passive.

The recombinant proteins may comprise at least one domain and in specific cases comprise two domains. In some embodiments, the domain comprises one or more ligand binding domains. The ligand binding domain may comprise a domain from a receptor, such as a domain from transforming growth factor beta receptor 2 (TGFBR2), FAS, interleukin 4 receptor (IL4R), and/or interleukin 10 receptor (IL10R). The ligand binding domain may comprise a domain that binds to transforming growth factor beta (TGF-0), FASL, and/or IL-4, respectively.

Embodiments of the disclosure include soluble recombinant proteins that are further defined as comprising (1) at least one inhibitory protein domain, wherein the inhibitory protein domain can bind to at least one immunosuppressive ligand, and (2) at least one activating protein domain. In specific cases, the inhibitory protein domain comprises an extracellular domain selected from the group consisting of TGFBR2, FAS, IL4R, IL10R, and a combination thereof. In some cases, the activating protein domain comprises at least one domain selected from the group consisting of IL-2, IL-7, IL-15, and a combination thereof.

In some embodiments, a domain on the recombinant protein comprises one or more activating domains. The activating domain may bind to one or more receptors on a cell, such as an immune cell, and activate an immune response upon binding, in at least some cases. Examples of activating domains include domains from IL-2, IL-7, and/or IL-15.

When the recombinant protein comprises two or more domains, the domains may or may not be linked with a protein linker. The linker may be any suitable linker for linking the domains, such as a G-S linker. In some embodiments, the linker comprises the sequence GGGSGGGGSGGGGSGGG (SEQ ID NO:1) or a functional derivative thereof.

Embodiments of the disclosure include recombinant proteins comprising at least one inhibitory moiety, including in some cases a moiety that is derived from a cellular receptor by truncation of the transmembrane domain from the receptor protein amino acid sequence. The inhibitory moiety may be fused to one or more fusion moieties. In specific cases, the inhibitory moiety is derived from any of TGFBR2, FAS, IL4R, or IL10R. In specific embodiments, the protein comprises a fusion moiety that comprises at least one polypeptide that is capable of inducing the activity of immune cells, and in specific cases they encompass any of biologically active forms of IL-2, IL-7 or IL-15.

In some embodiments, the recombinant protein is Fas Decoy (FD) and comprises the sequence of

(SEQ ID NO: 2)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSN

SEQ ID NO:2 may be encoded by SEQ ID NO:3, as follows:

(SEQ ID NO: 3)
ATGTTAGGTATCTGGACTCTGCTGCCTCTAGTGTTGACCTCTGTCGCTAG
ACTGAGCAGTAAATCAGTCAACGCTCAGGTGACTGATATTAACTCCAAAG
GGCTGGAGCTTCGGAAAACGGTGACAACAGTTGAGACCCAGAACCTGGAG
GGACTTCACCACGACGGTCAGTTTTGCCACAAACCTTGTCCCCCTGGCGA
ACGAAAAGCACGAGATTGCACTGTCAACGGAGACGAGCCAGACTGCGTCC
CCTGTCAGGAGGGCAAAGAGTACACTGATAAGGCCCACTTTTCCTCAAAG
TGCCGCCGCTGTAGGCTGTGTGACGAAGGCCACGGGCTCGAAGTTGAAAT
CAATTGCACGAGAACACAGAACACTAAGTGTCGTTGCAAGCCGAATTTCT
TTTGTAACAGCACAGTCTGCGAGCACTGCGACCCTTGTACGAAGTGTGAG
CACGGGATTATTAAGGAGTGTACCCTAACCTCCAATACCAAGTGCAAGGA
AGAAGGAAGCAGGAGCAACTGA

In some embodiments, the recombinant protein is mutant Fas Decoy (FA) comprising the following sequence:

(SEQ ID NO: 4)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CSRCRLCDEGHGLEVEINCTRTQNTKCRCKPNFFCNSTVCEHCDPCTKCE
HGIIKECTLTSNTKCKEEGSRSN

SEQ ID NO:4 may be encoded by SEQ ID NO:5 as follows:

(SEQ ID NO: 5)
ATGTTAGGTATCTGGACTCTGCTGCCTCTAGTGTTGACCTCTGTCGCTAG
ACTGAGCAGTAAATCAGTCAACGCTCAGGTGACTGATATTAACTCCAAAG
GGCTGGAGCTTCGGAAAACGGTGACAACAGTTGAGACCCAGAACCTGGAG
GGACTTCACCACGACGGTCAGTTTTGCCACAAACCTTGTCCCCCTGGCGA
ACGAAAAGCACGAGATTGCACTGTCAACGGAGACGAGCCAGACTGCGTCC
CCTGTCAGGAGGGCAAAGAGTACACTGATAAGGCCCACTTTTCCTCAAAG
TGCTCTCGCTGTAGGCTGTGTGACGAAGGCCACGGGCTCGAAGTTGAAAT
CAATTGCACGAGAACACAGAACACTAAGTGTCGTTGCAAGCCGAATTTCT
TTTGTAACAGCACAGTCTGCGAGCACTGCGACCCTTGTACGAAGTGTGAG
CACGGGATTATTAAGGAGTGTACCCTAACCTCCAATACCAAGTGCAAGGA
AGAAGGAAGCAGGAGCAACTGA

In some embodiments, the recombinant protein is mutant TGFβ Decoy comprising the following sequence:

(SEQ ID NO: 6)
MKWVTFLLLLFISGSAFSAAANGAVKFPQLCKFCDVRFSTCDNQKSCMSN
CSITSICEKPQEVCVAVWRKNDENITLETVCHDPKLPYHDFILEDAASPK
CIMKEKKKPGETFFMCSCSSDECNDNIIFSEEYNTSNPDGLGPVESSPGH
GLDTAAAGPEPSTRCELSPINASHPVQALMESFTVLSGCASHGTTGLPRE
VHVLNLRSTDQGPGQRQREVTLHLNPIASVHTHHKPIVFLLNSPQPLVWR
LKTERLAAGVPRLFLVSEGSVVQFPSGNFSLTAETEERNFPQENEHLLRW
AQKEYGAVTSFTELKIARNIYIKVGEDQVFPPTCNIGKNFLSLNYLAEYL
QPKAAEGCVLPSQPHEKEVHIIELITPSSNPYSAFQVDIIVDIRPAQEDP
EVVKNLVLILKSKKSVNWVIKSFDVKGNLKVIAPNSIGFGKESERSMTMT
KLVRDDIPSTQENLMKWALDAGYRPVTSYTMAPVANRFHLRLENNEEMRD
EEVHTIPPELRILLDPDKLPQLCKFCDVRFSTCDNQKSCMSNCSITSICE
KPQEVCVAVWRKNDENITLETVCHDPKLPYHDFILEDAASPKCIMKEKKK
PGETFFMCSCSSDECNDNIIFSEEYNTSNPD

SEQ ID NO:6 may be encoded by SEQ ID NO:7 as follows:

(SEQ ID NO: 7)
ATGAAATGGGTGACATTTCTGCTGCTGCTGTTTATCTCCGGTTCCGCTTT
TTCCGCCGCTGCCAACGGGGCCGTGAAATTTCCACAGCTGTGCAAGTTTT
GTGACGTACGGTTTTCCACCTGCGACAACCAAAAGTCTTGTATGTCTAAT
TGCAGTATCACCTCCATTTGTGAGAAGCCACAGGAGGTCTGCGTTGCCGT
ATGGCGGAAGAATGACGAAAACATTACACTTGAAACCGTGTGCCACGACC
CTAAACTTCCATACCATGACTTCATCCTGGAAGATGCCGCTAGTCCGAAG
TGCATTATGAAAGAGAAGAAAAAGCCCGGCGAGACATTCTTCATGTGTTC
CTGTTCTTCTGATGAGTGCAACGACAACATCATCTTCAGTGAGGAATATA
ACACAAGCAACCCTGACGGACTGGGCCCCGTGGAAAGTTCCCCAGGCCAC
GGCTTAGACACAGCAGCTGCAGGTCCTGAGCCCAGTACTCGATGCGAGCT
TAGTCCCATTAATGCTTCTCACCCCGTCCAAGCACTCATGGAGTCTTTCA
CAGTACTCAGCGGCTGCGCATCGCACGGCACTACCGGACTGCCCAGGGAG
GTCCATGTTTTGAATCTCCGCTCAACTGATCAGGGCCCAGGCCAGAGGCA
GCGAGAGGTGACTCTCCATCTGAATCCCATCGCAAGCGTCCATACACACC
ACAAGCCAATAGTTTTCCTGTTAAACTCTCCACAGCCACTAGTATGGAGA
CTCAAGACCGAGAGACTGGCTGCCGGGGTGCCTAGACTTTTCCTGGTCAG
TGAAGGTTCCGTGGTCCAGTTTCCTTCCGGCAACTTCAGCCTGACTGCTG
AGACCGAAGAGCGTAACTTCCCACAAGAGAATGAACATTTGCTGAGATGG
GCTCAAAAGGAATACGGCGCAGTAACCTCCTTTACGGAGCTGAAGATCGC
GCGCAACATCTATATTAAAGTAGGCGAGGACCAAGTCTTTCCTCCAACCT
GTAACATCGGAAAGAACTTTCTGTCGCTGAACTATCTGGCCGAATATCTG
CAACCTAAAGCCGCTGAGGGGTGCGTTTTACCATCTCAGCCCCATGAAAA
GGAGGTACATATCATAGAGTTAATAACCCCCAGTAGCAACCCATATTCCG
CCTTTCAGGTTGACATAATCGTCGATATTAGACCAGCCCAGGAAGACCCC
GAAGTGGTCAAAAACCTTGTACTCATCTTGAAATCAAAGAAGAGCGTGAA
TTGGGTGATCAAAAGCTTCGACGTTAAAGGGAACCTGAAAGTGATTGCTC
CTAACAGCATCGGCTTCGGGAAGGAGTCGGAGCGGAGTATGACGATGACG
AAACTAGTGCGGGATGATATTCCATCAACGCAAGAGAACTTAATGAAATG
GGCACTGGATGCAGGTTATAGACCAGTGACCAGCTATACCATGGCCCCCG
TAGCCAATAGATTCCACCTAAGGCTAGAAAACAACGAGGAAATGCGCGAC
GAGGAGGTCCATACAATCCCTCCAGAGCTGAGGATCTTGTTGGACCCAGA
CAAGCTGCCACAGTTGTGTAAGTTCTGTGATGTCCGCTTTTCAACATGCG
ATAACCAGAAGAGCTGCATGTCAAATTGCAGTATTACATCAATTTGTGAA
AAGCCCCAGGAGGTATGTGTGGCCGTTTGGAGGAAAAACGATGAGAACAT
AACATTGGAGACGGTGTGTCATGATCCCAAACTGCCGTATCATGATTTCA
TACTGGAGGATGCCGCCTCCCCCAAATGCATCATGAAGGAAAAGAAAAAG
CCTGGAGAAACTTTTTTCATGTGCAGCTGCTCAAGTGACGAATGCAATGA
TAACATTATCTTCAGCGAAGAATATAACACGTCAAATCCTGACTAG

In some embodiments, the recombinant protein is mutant TGFβ sensing decoy (T3zR) comprising the following sequence:

(SEQ ID NO: 8)
MGRGLLRGLWPLHIVLWTRIASTIPPHVQKSVNNDMIVTDNNGAVKFPQL
CKFCDVRFSTCDNQKSCMSNCSITSICEKPQEVCVAVWRKNDENITLETV
CHDPKLPYHDFILEDAASPKCIMKEKKKPGETFFMCSCSSDECNDNIIFS
EEYNTSNPDLLLVIFQTTTPAPRPPTPAPTIASQPLSLRPEACRPAAGGA
VHTRGLDFACDIYIWAPLAGTCGVLLLSLVITLYCRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPREGR
GSLLTCGDVEENPGP

SEQ ID NO:8 may be encoded by SEQ ID NO:9, as follows:

(SEQ ID NO: 9)
ATGGGTCGAGGACTTCTAAGAGGTCTGTGGCCACTTCATATTGTTCTGTG
GACGCGTATCGCTTCTACAATACCACCTCACGTTCAGAAGTCAGTTAATA
ACGACATGATTGTCACTGACAACAACGGTGCAGTCAAGTTTCCACAACTG
TGTAAATTTTGTGATGTGAGATTTTCCACCTGTGACAACCAGAAATCCTG
CATGAGCAACTGCAGCATCACCTCCATCTGTGAGAAGCCACAGGAAGTCT
GTGTGGCTGTATGGAGAAAGAATGACGAGAACATAACACTAGAGACAGTT
TGCCATGACCCCAAGCTCCCCTACCATGACTTTATTCTGGAAGATGCTGC
TTCTCCAAAGTGCATTATGAAGGAAAAAAAAAAGCCTGGTGAGACTTTCT
TCATGTGTTCCTGTAGCTCTGATGAGTGCAATGACAACATCATCTTCTCA
GAAGAATATAACACCAGCAATCCTGACTTGTTGCTAGTCATATTTCAAAC
CACAACTCCTGCGCCACGGCCTCCGACGCCCGCTCCTACGATAGCCAGTC
AGCCCTTGTCATTGCGACCCGAAGCCTGTCGGCCTGCAGCTGGAGGTGCA
GTGCACACGAGGGGGCTGGACTTCGCCTGTGATATCTACATCTGGGCGCC
CTTGGCCGGGACTTGTGGGGTCCTTCTCCTGTCACTGGTTATCACCCTTT
ACTGCAGAGTGAAGTTCAGCAGGAGCGCAGACGCCCCCGCGTACCAGCAG
GGCCAGAACCAGCTCTATAACGAGCTCAATCTAGGACGGAGAGAGGAGTA
CGATGTTTTGGACAAGAGACGTGGCCGGGACCCTGAGATGGGGGGAAAGC
CGAGAAGGAAGAACCCTCAGGAAGGCCTGTACAATGAACTGCAGAAAGAT
AAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGG
ACACCTACGACGCCCTTCACATGCAGGCCCTGCCCCCTCGCGAGGGCAGA
GGAAGTCTACTTACATGCGGTGATGTGGAAGAGAATCCCGGCCCTTAG

In some embodiments, the recombinant protein is Fas Decoy-IL-15 Fusion (FD+15) comprising the following sequence:

(SEQ ID NO: 10)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNTRGGGSGGGGPWNWVNVISDLKKIEDL
IQSMHIDATLYTESDVHPSCKVTAMKCFLLELQVISLESGDASIHDTVEN
LIILANNSLSSNGNVTESGCKECEELEEKNIKEFLQSFVHIVQMFINTS

SEQ ID NO:10 may be encoded by SEQ ID NO:11 as follows:

(SEQ ID NO: 11)
ATGTTAGGTATCTGGACTCTGCTGCCTCTAGTGTTGACCTCTGTCGCTAG
ACTGAGCAGTAAATCAGTCAACGCTCAGGTGACTGATATTAACTCCAAAG
GGCTGGAGCTTCGGAAAACGGTGACAACAGTTGAGACCCAGAACCTGGAG
GGACTTCACCACGACGGTCAGTTTTGCCACAAACCTTGTCCCCCTGGCGA
ACGAAAAGCACGAGATTGCACTGTCAACGGAGACGAGCCAGACTGCGTCC
CCTGTCAGGAGGGCAAAGAGTACACTGATAAGGCCCACTTTTCCTCAAAG
TGCCGCCGCTGTAGGCTGTGTGACGAAGGCCACGGGCTCGAAGTTGAAAT
CAATTGCACGAGAACACAGAACACTAAGTGTCGTTGCAAGCCGAATTTCT
TTTGTAACAGCACAGTCTGCGAGCACTGCGACCCTTGTACGAAGTGTGAG
CACGGGATTATTAAGGAGTGTACCCTAACCTCCAATACCAAGTGCAAGGA
AGAAGGAAGCAGGAGCAACTGAACGCGTGGCGGAGGTTCAGGAGGAGGTG
GCCCATGGAATTGGGTCAACGTTATTAGTGACCTAAAAAAGATTGAAGAC
CTGATTCAGTCTATGCACATCGACGCAACCCTCTACACAGAGTCGGACGT
GCACCCTAGCTGCAAGGTTACGGCAATGAAGTGCTTCCTTCTCGAACTTC
AGGTTATTTCACTTGAATCCGGTGACGCTTCGATTCACGACACTGTCGAA
AATCTGATAATACTCGCCAACAACTCCCTTAGCTCGAACGGGAACGTAAC
CGAGAGCGGGTGCAAAGAGTGCGAAGAGTTAGAGGAAAAAAATATTAAGG
AATTTCTGCAGTCCTTTGTGCATATTGTCCAAATGTTTATAAATACTAGC
TGA

In some embodiments, the recombinant protein is Mutant Fas Decoy-IL-15 Fusion (FΔ+15) comprising the following sequence:

(SEQ ID NO: 12)
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CSRCRLCDEGHGLEVEINCTRTQNTKCRCKPNFFCNSTVCEHCDPCTKCE
HGIIKECTLTSNTKCKEEGSRSNTRGGGSGGGGPWNWVNVISDLKKIEDL
IQSMHIDATLYTESDVHPSCKVTAMKCFLLELQVISLESGDASIHDTVEN
LIILANNSLSSNGNVTESGCKECEELEEKNIKEFLQSFVHIVQMFINTS

SEQ ID NO:12 may be encoded by SEQ ID NO:13 as follows:

(SEQ ID NO: 13)
ATGTTAGGTATCTGGACTCTGCTGCCTCTAGTGTTGACCTCTGTCGCTAG
ACTGAGCAGTAAATCAGTCAACGCTCAGGTGACTGATATTAACTCCAAAG
GGCTGGAGCTTCGGAAAACGGTGACAACAGTTGAGACCCAGAACCTGGAG
GGACTTCACCACGACGGTCAGTTTTGCCACAAACCTTGTCCCCCTGGCGA
ACGAAAAGCACGAGATTGCACTGTCAACGGAGACGAGCCAGACTGCGTCC
CCTGTCAGGAGGGCAAAGAGTACACTGATAAGGCCCACTTTTCCTCAAAG
TGCTCTCGCTGTAGGCTGTGTGACGAAGGCCACGGGCTCGAAGTTGAAAT
CAATTGCACGAGAACACAGAACACTAAGTGTCGTTGCAAGCCGAATTTCT
TTTGTAACAGCACAGTCTGCGAGCACTGCGACCCTTGTACGAAGTGTGAG
CACGGGATTATTAAGGAGTGTACCCTAACCTCCAATACCAAGTGCAAGGA
AGAAGGAAGCAGGAGCAACTGAACGCGTGGCGGAGGTTCAGGAGGAGGTG
GCCCATGGAATTGGGTCAACGTTATTAGTGACCTAAAAAAGATTGAAGAC
CTGATTCAGTCTATGCACATCGACGCAACCCTCTACACAGAGTCGGACGT
GCACCCTAGCTGCAAGGTTACGGCAATGAAGTGCTTCCTTCTCGAACTTC
AGGTTATTTCACTTGAATCCGGTGACGCTTCGATTCACGACACTGTCGAA
AATCTGATAATACTCGCCAACAACTCCCTTAGCTCGAACGGGAACGTAAC
CGAGAGCGGGTGCAAAGAGTGCGAAGAGTTAGAGGAAAAAAATATTAAGG
AATTTCTGCAGTCCTTTGTGCATATTGTCCAAATGTTTATAAATACTAGC
TGA

Compositions encompassed herein include nucleic acids and/or genetic viral or non-viral vectors comprising sequences encoding at least one of the recombinant proteins disclosed herein. The nucleic acids and/or genetic vectors may encode sequences for transient expression of the recombinant protein. The nucleic acids and/or genetic vectors may encode sequences for constitutive expression of the recombinant protein. The nucleic acids and/or genetic vectors may encode sequences for inducible expression of the recombinant protein. The nucleic acids and/or genetic vectors may encode sequences for tissue-specific expression of the recombinant protein.

Compositions encompassed herein include a cell comprising at least one of the recombinant proteins, nucleic acids, and/or genetic vectors. disclosed herein. The cells may be any cell type including T lymphocytes, natural killer (NK) cells, NK T cells, B cells, myeloid cells including macrophages, dendritic cells, mesenchymal stem cells, and/or fibroblasts. The cell may be genetically modified to express the recombinant protein and/or express one or more other heterologous proteins such as one or more antigen receptors. In specific embodiments, the antigen receptors target one or more cancer antigens.

Compositions encompassed herein comprise a therapeutic composition comprising a recombinant protein, a nucleic acid, a genetic vector, and/or a cell of the disclosure.

### A. Decoy Proteins

In embodiments herein, the decoy protein may be produced by recombinant DNA/exogenous expression methods or produced by solid-phase peptide synthesis (SPPS) or other in vitro methods. In particular embodiments, there are isolated nucleic acid segments and recombinant vectors incorporating nucleic acid sequences that encode a polypeptide (e.g., a decoy protein or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments, the size of a decoy protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, 650, 700, 750, 800, 850, 900, 950, 1000 or more contiguous amino acids or nucleic acids, or any range derivable therein, of SEQ ID NOs 2-13.

In some embodiments, certain amino acids of one or more of the domains in the decoy protein may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable change of their biological utility or activity. It is also contemplated by the inventors that various changes may be made in the DNA sequences of genes that encode proteins with predictable modulation of their biological utility or activity.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### B. Nucleic Acids

In certain embodiments, there are decoy protein-encoding polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy.

### C. Therapeutic Compositions

In certain aspects, the decoy compositions or agents for use in the methods herein (including cells that express the decoy proteins) are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as a tumor or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### III. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, a decoy therapy and a non-decoy therapy are administered substantially simultaneously. In some embodiments, the decoy therapy and a non-decoy therapy are administered sequentially. In some embodiments, the decoy therapy is administered before administering the non-decoy therapy. In some embodiments, the decoy therapy is administered after administering the non-decoy therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, the decoy therapy comprises a recombinant protein, a nucleic acid encoding for the recombinant protein, a vector comprising the nucleic acid encoding for the recombinant protein, or a cell comprising the recombinant protein, a nucleic acid encoding for the recombinant protein, or a vector comprising the nucleic acid encoding for the recombinant protein. In some embodiments, a single dose of the recombinant protein therapy is administered. In some embodiments, multiple doses of the recombinant protein are administered. In some embodiments, the recombinant protein is administered at a dose of between 1 mg/kg and 5000 mg/kg. In some embodiments, the recombinant protein is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some embodiments, a single dose of the non-decoy therapy is administered. In some embodiments, multiple doses of the non-decoy therapy are administered. In some embodiments, the non-decoy therapy is administered at a dose of between 1 mg/kg and 100 mg/kg. In some embodiments, the non-decoy therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

In embodiments wherein any kind of cells are utilized as the pharmaceutical composition administered to the individual in need thereof, the dose may be 1×10⁷ to 1×10⁹ cells per m² and any range derivable therein.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### IV. Methods of Use

Embodiments of the present disclosure concern methods for the use of decoy proteins or cells that express the decoy proteins, such as immune effector cells. The proteins or cells provided herein may be used for treating or preventing a medical disease or disorder. The method includes administering to the subject a therapeutically effective amount of the proteins or cells, thereby treating or preventing the disorder in the subject. In certain embodiments of the present disclosure, cancer is treated at least in part by using compositions that elicit an immune response.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments of the present disclosure, immune cells are delivered to an individual in need thereof, such as an individual that has cancer. The cells then enhance the individual's immune system to attack the cancer cells. In some cases, the individual is provided with one or more doses of the decoy proteins or immune cells expressing them. In cases where the individual is provided with two or more doses of the decoy proteins or immune cells expressing them, the duration between the administrations may be 1, 2, 3, 4, 5, 6, 7, or more days, or 1, 2, 3, or 4 weeks, or 1, 2, 3, 4, 5, 6, 7, 8, 9, 10, 11, 12, or more months.

When cells expressing the decoy proteins are utilized, suitable doses for a therapeutic effect may be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

The decoy protein-expressing immune cells encompassed herein have many potential uses, including experimental and therapeutic uses. In particular, it is envisaged that such cell populations will be extremely useful in suppressing undesirable or inappropriate immune responses. In such methods, a small number of immune cells are removed from a patient and then manipulated to express the decoy protein(s) and optionally expanded ex vivo before reinfusing them into the patient. A therapeutic method could comprise obtaining immune cells from an individual; manipulating the cells to express the decoy protein(s); optionally expanding the immune cells ex vivo; and administering the cells to a recipient that is different than the individual from which the immune cells were originally obtained.

A pharmaceutical composition of the present disclosure can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

In certain embodiments, the decoy protein-expressing immune cells are administered in combination with a second therapeutic agent. For example, the second therapeutic agent may comprise T cells, an immunomodulatory agent, a monoclonal antibody, or a chemotherapeutic agent. In non-limiting examples, the immunomodulatory agent is lenolidomide, the monoclonal antibody is rituximab, ofatumab, or lumiliximab, and the chemotherapeutic agent is fludarabine or cyclophosphamide.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

Desirably an effective amount or sufficient number of the decoy proteins or decoy protein-expressing immune cells is present in the composition and introduced into the subject such that long-term, specific, anti-tumor responses are established to reduce the size of a tumor or eliminate tumor growth or regrowth than would otherwise result in the absence of such treatment. Desirably, the amount of proteins or cells administered to the subject causes a 10%, 20%, 30%, 40%, 50%, 60%, 70%, 80%, 90%, 95%, 98%, or 100% decrease in tumor size when compared to otherwise same conditions wherein the immune cells are not present.

Accordingly, the amount of immune cells administered should take into account the route of administration and should be such that a sufficient number of the immune cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of immune cells desirably should be sufficient to provide in the subject being treated at least from about 1×10⁶ to about 1×10⁹ immune cells, even more desirably, from about 1×10⁷ to about 5×10⁸ immune cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of decoy proteins or decoy protein-expressing immune cells to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on inter-individual differences in pharmacokinetics, drug disposition, and metabolism. One skilled in the art readily can make any necessary adjustments in accordance with the exigencies of the particular situation.

Embodiments of the disclosure include methods for modifying cells with a genetic vector to express the soluble decoy proteins. In specific embodiments, the genetic vector encompasses any of viral and non-viral vectors that are capable of modifying the cells to permanently express the decoy protein. In specific cases, the genetic vector encompasses any of viral and non-viral vectors that are capable of modifying the cells to transiently express the decoy protein. The modified cells encompass any of T lymphocytes, natural killer cells, macrophages, mesenchymal stromal cells or fibroblasts from either autologous or allogenic sources. In some cases, the modified cells are also modified to express one or more additional proteins.

Embodiments of the disclosure include methods for administration of an effective amount of the decoy protein to treat cancer patients, including systemic or local administration of a therapeutically effective amount of a composition comprising the decoy proteins. An effective dose of the modified cells may be given in single or multiple doses. The method may utilize combination with other therapies that activate immune responses directly or indirectly.

### V. Kits

In some embodiments, a kit that can include, for example, one or more media and components for the production of decoy proteins and/or immune cells is provided. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: CAR T Cells are Susceptible to Tumor-Expressed FasL Mediated Apoptosis

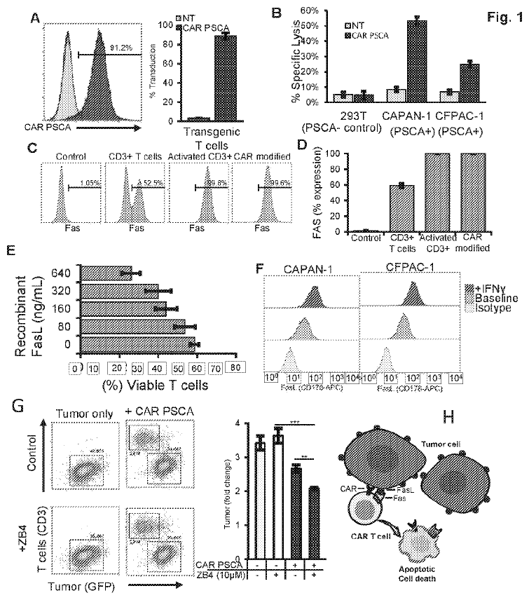

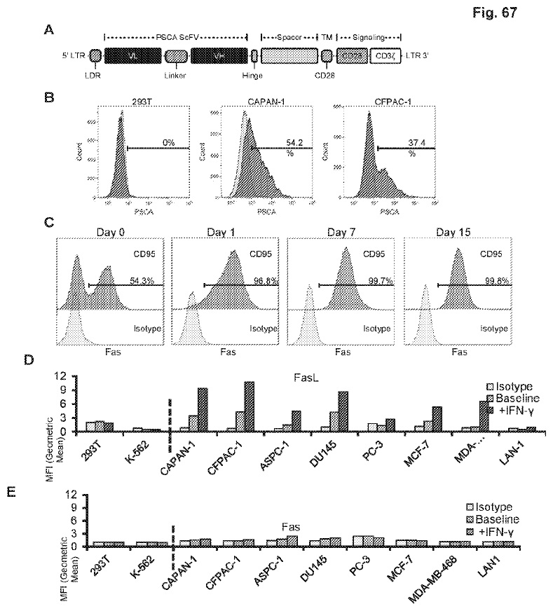

In order to target PSCA, we have previously engineered a second-generation human codon-optimized CAR containing an anti-PSCA scFv (clone: 2B3), an IgG2 hinge-CH3 domain, and a CD28 costimulatory endodomain (FIG. 67A). We were able to efficiently transduce T cells derived from healthy donor PBMCs to express this CAR by retroviral transduction (FIG. 1A—histogram-representative donor, bar graph-summary data), which enabled specific lysis of PSCA-expressing target cells as assessed in a short-term (5-hr) 51Cr-release assay. As shown in FIG. 1B, anti-tumor activity against PSCA+ pancreatic cancer cell lines CAPAN-1 and CFPAC-1 correlating with their PSCA expression levels (FIG. 67B) was observed with minimal activity against PSCA- 293T cells.

To better understand the impact of Fas-FasL pathway on CAR T cells, we first explored the dynamics of Fas expression. At baseline approximately half of the unstimulated CD3+ T cells in donor PBMC expressed Fas and within 24 hr of activation with anti-CD3 and anti-CD28 antibodies, virtually all T cells expressed Fas which was maintained after retroviral transduction for at least 15 days (FIG. 1C and FIG. 67C). Importantly, this Fas upregulation conferred T cells sensitive to FasL-mediated apoptosis, as illustrated in FIG. 1E, where CAR T cell viability (assessed by Annexin-V and 7-AAD staining) decreased upon exposure to increasing concentrations of recombinant FasL. To understand the susceptibility of T cells to tumor ‘counterattack’, we measured FasL on a range of tumor cell lines representing multiple solid tumors [e.g. pancreatic (CAPAN-1 and CFPAC-1), prostate, and breast cancer] and found that almost all expressed FasL at baseline, which was further upregulated upon IFNγ exposure (FIG. 1F and FIG. 67D). Interestingly, Fas expression on these lines was minimal/absent under the same conditions (FIG. 67E). To test the impact of tumor-expressed FasL on CAR T cells, we performed CAPAN-1 tumor-CAR PSCA T cell co-cultures in the absence or presence of a Fas-blocking antibody (clone: ZB4, 10 uM). Quantification of residual tumor cells 3-days post co-culture demonstrated improved anti-tumor activity of CAR PSCA T cells in the presence of the blocking antibody as shown in FIG. 1F (density plot-representative donor, bar graph-summary data). Taken together, this data suggests that interactions between Fas on CAR T cells and FasL expressed by tumor cells can lead to the suppression of effector T cells and consequently result in suboptimal tumor control (FIG. 1G).

### Example 2: Developing an engineered Fas decoy receptor to sequester FasL

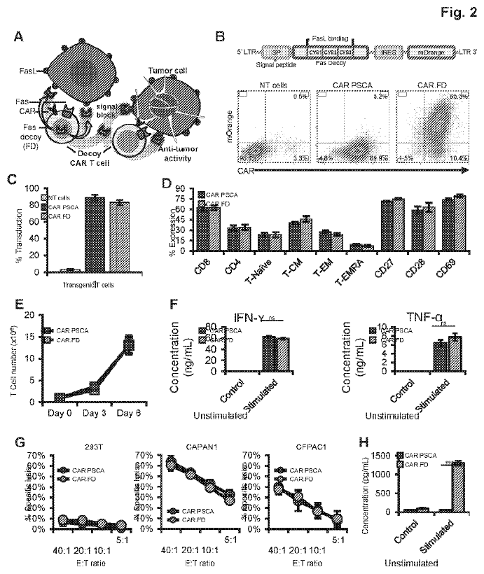

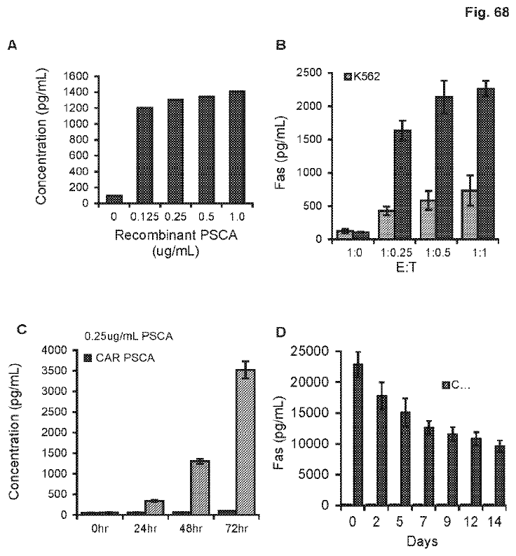

To protect T cells from FasL-mediated suppression, we developed a secretable decoy molecule (FD) to prevent Fas engagement (FIG. 2A). Our retroviral vector encodes the extracellular portion of Fas linked with mOrange via an IRES sequence to facilitate transgene detection (FIG. 2B) and can be co-expressed with the CAR at high efficiency (FIG. 2B —representative donor; and FIG. 2C—summary data). To evaluate the impact of transgenic FD expression, we performed flow cytometric analysis on our cell populations, which revealed that both CAR PSCA and CAR.FD T cells were similar with respect to CD4:CD8 distribution as well as expression of activation (e.g. CD69, CD27, and CD28), and memory markers (naïve/undifferentiated −CCR7+CD45RO−, central memory −CCR7+CD45RO+, effector memory—CCR7−CD45RO+, and effector/terminally differentiated—CCR7-CD45RO−) (FIG. 2D). Next, to assess if FD expression affected cell expansion upon antigen exposure, we stimulated CAR and CAR.FD T cells with irradiated artificial antigen presenting cells (K562 cells engineered to express PSCA—K562-PSCA) and found no significant difference (p=0.96) in growth over the course of 6 days (FIG. 2E). Both T cell populations also secreted comparable amounts of effector cytokines IFN-γ and TNF-α upon stimulation with K562-PSCA cells as shown in FIG. 2F. Next, we confirmed that the CAR-mediated cytolytic activity of CAR.FD T cells was unaffected by FD in a 51Cr-release assay, where transgenic cells lysed PSCA+ tumor targets CAPAN-1 and CFPAC-1 at levels similar to CAR-only modified T cells at various E:T ratios (FIG. 2G). Finally, to confirm that CAR.FD cells produced the decoy, T cells were stimulated with plate-bound recombinant PSCA and 48-hrs later supernatant was harvested for Fas quantification by ELISA. As shown in FIG. 2H, only dual-transgenic T cells produced FD, and the levels produced correlated with the magnitude (recombinant PSCA—FIG. 68A, K562-PSCA—FIG. 68B) and duration of stimulation (FIG. 68C). We also tested the stability of FD at 37° C. by measuring FD in medium obtained from FD-transduced 293T (293T-FD), which suggested that the half-life of this soluble decoy molecule is approximately 12 days (FIG. 68D). Taken together, these results demonstrate the feasibility of engineering CAR T cells to produce FD.

### Example 3: Fas Decoy-Engineered T Cells Exhibit Superior Function in Presence of FasL

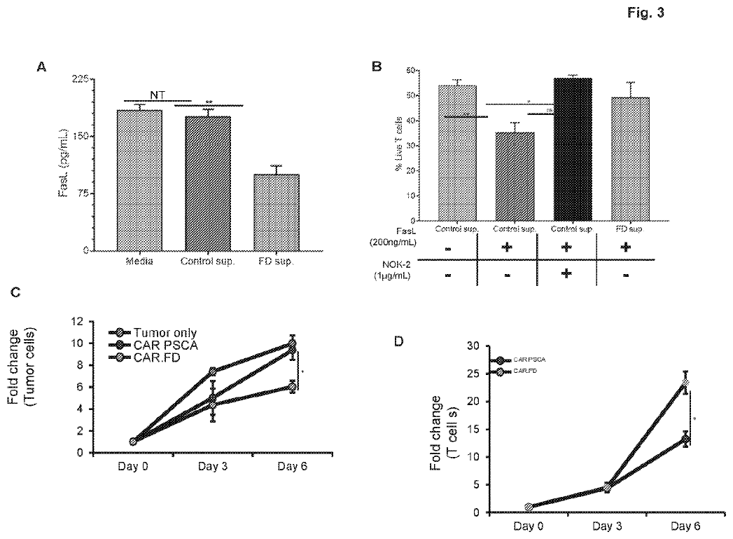

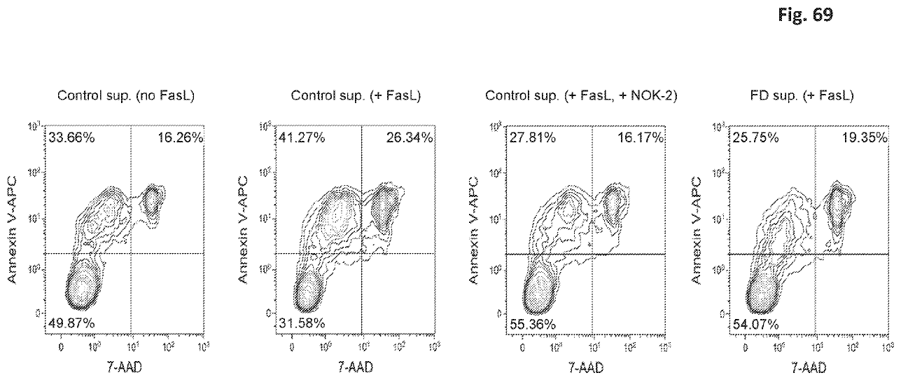

To evaluate the functional characteristics of the decoy and its impact on transgenic T cells we first examined the FasL neutralizing properties of FD using an ELISA-based neutralization assay. As shown in FIG. 3A, after 1-hr incubation FasL detection in 293T-FD conditioned medium was significantly reduced compared to the control (293T) supernatant or fresh medium, suggesting sequestration of FasL by the decoy. To investigate if the neutralizing effects of FD was sufficient to protect T cells from FasL-induced apoptotic death, CAR PSCA T cells were cultured with recombinant FasL (200 ng/mL) and either control (293T) or decoy-containing supernatant (293T-FD). Control supernatant supplemented with FasL-blocking antibody (NOK-2) was used as an additional control. After 24 hr of exposure, cell viability was assessed by Annexin V and 7-AAD staining. As shown in FIG. 3B (summary data) and FIG. 69 (representative donor), culture with the decoy-containing supernatant was associated with superior T cell viability, similar to NOK-2 containing supernatant and supernatant lacking recombinant FasL, whereas viability was markedly reduced in control supernatant containing FasL. This improved T cell survival translated to superior anti-tumor activity as measured in a 6-day coculture experiment where CAPAN-1 tumor cells were mixed with either CAR PSCA or CAR.FD cells at a low E:T (1:10) to recreate the challenging conditions of the TME. As shown in FIG. 3C, tumor cells expanded rapidly when left untreated and while CAR T cells transiently controlled tumor growth between days 0 and 3, tumor cells eventually outgrew by day 6. In contrast, CAR.FD T cells exhibited enhanced anti-tumor activity indicated by fewer residual tumor cells on day 6. This improved cytolytic activity of CAR.FD cells correlated with their enhanced survival/expansion compared to CAR PSCA T cells (FIG. 3D). In summary, these results from our in vitro functional assessment of FD demonstrated that the decoy receptor interacts with and neutralizes FasL, resulting in improved T cell survival and anti-tumor effects upon encounter with FasL-expressing tumors.

### Example 4: Combining the Fas Decoy and IL-15 Fusion Protein (FD.15) to Enhance T Cell

### Activity

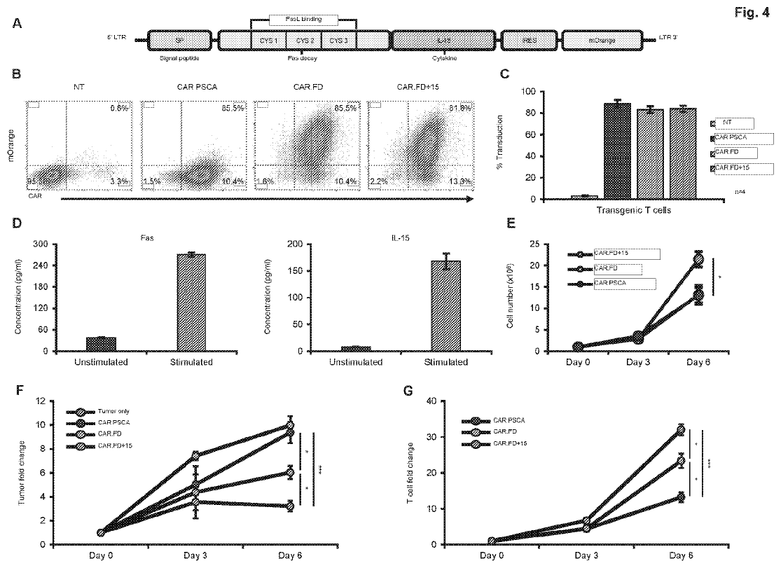

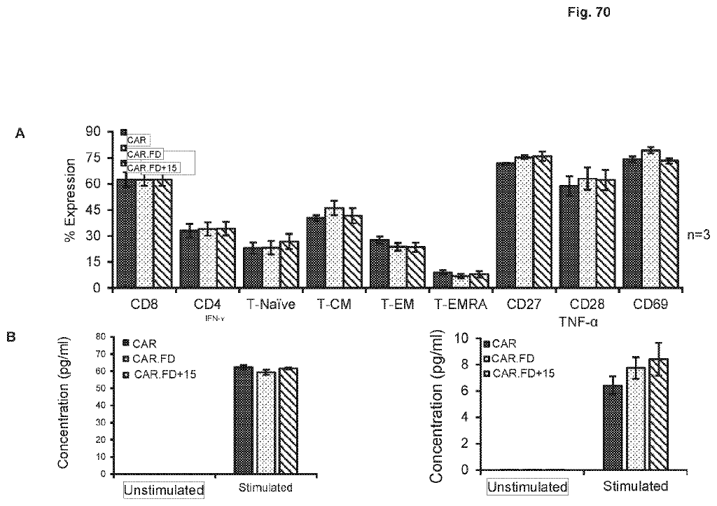

To investigate whether the incorporation of a growth-promoting cytokine signal could further augment the anti-tumor effects of decoy-modified CAR T cells, we developed a new retroviral construct encoding FD and human IL-15 cytokine fusion molecule (FD+15 -FIG. 4A). CAR PSCA T cells were transduced to express co-express FD+15 at levels comparable to FD alone, as shown in FIG. 4B (representative donor) and FIG. 4C (summary data). Next, to assess the effects of FD+15 expression on T cells, we evaluated the phenotype and effector cytokine production of CAR.FD+15 cells, which was similar CAR only and CAR.FD cells (FIG. 70A, 70B). Next, to confirm that the fusion molecule was secreted, we collected supernatant from CAR.FD+15 T cells cultured with or without recombinant PSCA antigen for 48 hrs and measured both soluble Fas (FD) and IL-15 by ELISA. As illustrated in FIG. 4D, T cells secreted both Fas decoy and IL-15 upon activation, whereas minimal quantities were detected in the absence of activation. When stimulated with K562-PSCA cells, CAR.FD+15 T cells demonstrated superior expansion compared to CAR PSCA or CAR.FD cells (FIG. 4E), likely due to transgenic IL-15. To assess if co-expression of FD+15 with the CAR further enhanced T cell effector function upon exposure to FasL+ tumor cells, we compared the anti-tumor activity and expansion of CAR only, CAR.FD, and CAR.FD+15 engineered cells following co-culture with CAPAN-1 cells (E:T of 1:10). As shown in FIG. 4F, tumor cell numbers increased rapidly when left untreated, and CAR PSCA T cells were only able to transiently slow tumor growth. While CAR.FD T cells exhibited improved anti-tumor effects compared to CAR-only modified cells, CAR.FD+15 T cells exhibited the greatest anti-tumor activity of all treatment conditions. This enhanced tumor-killing activity correlated with superior survival/expansion of CAR.FD+15 T cells compared with both CAR PSCA and CAR.FD T cells, as demonstrated in FIG. 4G. Taken together, these results indicate that the FD+15 fusion molecule further potentiates the anti-tumor activity and survival/expansion of T cells exposed to FasL+ tumors.

### Example 5: Decoy Engineered CAR T Cells Demonstrate Enhanced In Vivo Anti-Tumor Activity

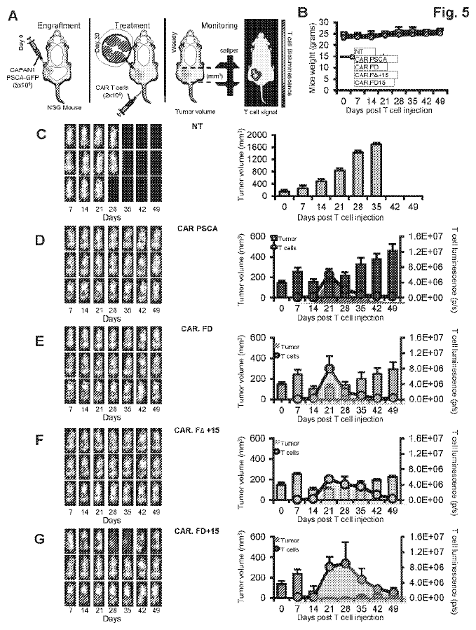

To assess the in vivo activity of decoy-engineered T cells, we utilized a xenograft model of pancreatic cancer in which NSG mice were engrafted with 5×106 CAPAN1 cells subcutaneously (s.c.) in the left flank followed by an intravenous injection (i.v.) (single dose) of 3×106 GFP FFLuc-labeled T cells once the tumor reached approximately 150mm3. An additional cytokine-only control for this experiment included T cells modified with a construct encoding FΔ+15 -a mutant Fas incapable of binding to FasL and functional IL-15 fusion. Cell expansion and persistence was measured by bioluminescence imaging while tumor volume was monitored by caliper measurement (FIG. 5A). To first assess the safety profile of decoy-modified T cells in vivo, we monitored mouse weight over the course of the experiment and additionally routinely examined animals for evidence of ruffled coat, hair-loss, and/or skin redness. As shown in FIG. 5B mouse weight remained stable in all groups, irrespective of treatment and even during peak T cell expansion (3-4 weeks post T cell injection). Furthermore, no other manifestations of toxic side-effects were noted, indicating the safety of the decoy. T cells localized to the tumor within 7 days of infusion in all animals except recipients of non-transduced (NT) T cells, as assessed by bioluminescence imaging (FIGS. 5C, D, E, F, and G-representative mice images on the left). However, compared to the CAR-only group (FIG. 5D, line graph), CAR.FD-treated mice showed improved T cell expansion compared with CAR only and CAR.FΔ+15 T cells, the latter exhibited improved persistence (FIG. 5E, day 21; FIG. 5F). However, CAR.FD+15 T cells, which were protected from FasL and also received IL-15 cytokine support, exhibited superior expansion and persistence compared to all treatment groups (FIG. 5G, line graph), resulting in robust and durable anti-tumor effects as illustrated in FIG. FIGS. 5C, D, E, F, and G (bar graphs). In summary, results from our in vivo study indicate that decoy-engineered CAR T cells localize to the tumor and mediate their cytotoxic potential at disease sites absent evidence of systemic toxicity. Furthermore, protection from the TME via FasL and the growth-promoting effects of IL-15 provided by the FD+15 fusion molecule resulted in greater T cell proliferation and functional persistence at the tumor leading to robust anti-tumor activity.

### Example 6: Decoy-Engineered T Cells Promote Activity of Bystander Effector Cells

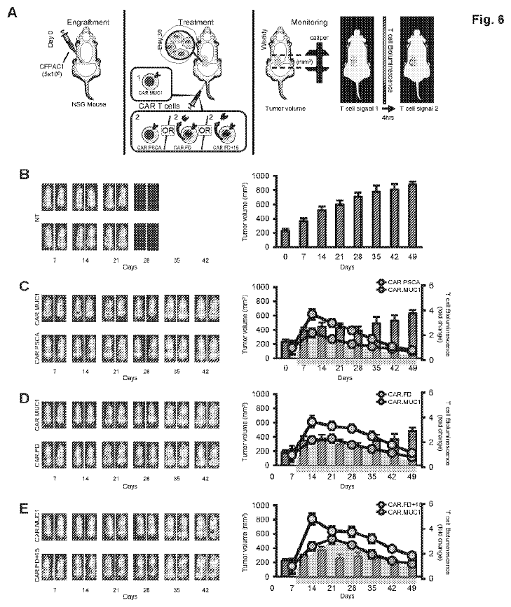

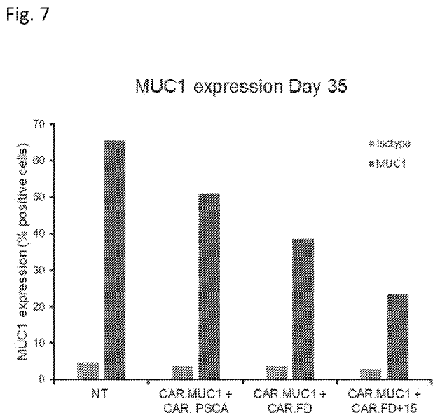

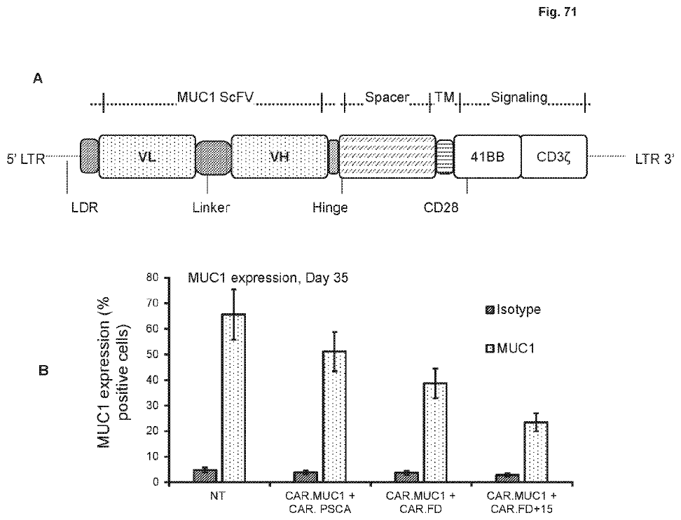

Certain embodiments herein focus on assessing the impact of transgenic decoy expression on cells transduced with the construct. However, the decoy molecule is a secreted compound that, in some embodiments, also provides benefit to neighboring (non-transgenic) bystander cells that might also be able to mediate anti-tumor effects if released from the suppressive effects of the tumor microenvironment. To this point our efforts have focused on assessing the impact of transgenic decoy expression on the transduced cells. However, the decoy molecule is a secreted compound that should theoretically also provide benefit to non-transgenic bystander cells. Thus, to address this potential “bystander benefit”, we engrafted NSG mice with the pancreatic cancer cell line CFPAC-1 and subsequently treated these animals with a 1:1 mix (2×106 of each) of CAR MUC1 T cells (which served as bystander cells) and either CAR PSCA, CAR.FD, or CAR.FD+15 T cells. To track the fate of each T cell type in vivo, CAR.MUC1 cells were co-transduced with click-beetle luciferase (CBG99) while CAR PSCA T cells were labeled with a mutant firefly luciferase (Akaluc) while tumor volume was measured by calipers (FIG. 6A). As illustrated in FIGS. 6B, C, D, and E (representative mice images on the left), T cell localization at the tumor was seen in all animals, with greater expansion of CAR.PSCA/CAR.FD/CAR.FD+15-expressing cells compared to bystander cells (FIGS. 6C, D, and E, line graphs). However, CAR.MUC1 T cell expansion in the animals co-administered with CAR.FD surpassed that seen in animals co-injected with CAR PSCA only T cells, and expansion was further improved when delivered with CAR.FD+15 T cells. This data supports the potential for bystander protection, which provides additive anti-tumor effects in vivo (CAR.MUC1+ CAR.FD—FIG. 6D; CAR.MUC1+ CAR.FD+15 T cells—FIG. 6E, bar graphs). We ascribe this benefit to enhanced CAR.MUC1 T cell activity since tumors resected 5-weeks post T cell infusion had fewer residual MUC1+ tumor cells in mice treated with CAR.MUC1+ CAR.FD and CAR.MUC1+ CAR.FD+15 treatments vs CAR.MUC1+ CAR.PSCA or groups, as shown in FIG. 71A. Taken together, these results support the tumor-localized bystander benefit of the decoy.

### Example 7: Fas Decoy Strategies

Redirecting the cellular immune response against cancer by engineering T cells to express CARs with specificities against tumor associated antigens has resulted in remarkable clinical success, particularly in patients with CD19+ malignancies. Unfortunately, solid tumors have proven more challenging to treat due to a number of factors including inhibitory mechanisms deployed by the tumor to restrict T cell persistence and limit their cytolytic function. These include upregulation of checkpoint molecules (e.g., PD-L1, CTLA-4), production of suppressive cytokines (e.g., IL-4, TGFβ), and expression of death receptor ligands such as FasL—an immunomodulatory molecule that induces apoptotic cell death upon engagement with its receptor—Fas, expressed by activated T cells. In this study, we show the benefit of protecting T cells from tumor-induced FasL-mediated apoptosis with our novel Fas decoy. Using CAR PSCA T cells in a FasL+ pancreatic cancer model system, we demonstrate that co-expression of both the CAR and decoy produce superior T cell expansion and anti-tumor effects both in vitro and in vivo. Additionally, the provision of additional cytokine support by employing a bi-functional fusion molecule of decoy and IL-15 cytokine further improved T cell proliferation and persistence and potentiated anti-tumor activity in a FasL-rich tumor environment. Finally, we showcase the paracrine immune-enhancing properties of our decoy molecule in a second pancreatic tumor (CFPAC-1) model where we demonstrate the beneficial effects conferred to bystander effectors.

Given the major detrimental impact of tumor-expressed FasL on adoptively transferred T cells, our group and others have previously explored genetic engineering strategies to render T cells “immune” to both tumor-FasL and activation induced cell death (AICD) by ablating Fas in T cells. For example, Dotti and colleagues used a siRNA to knock-down Fas on EBV-specific T cells to render cells resistant to EBV-associated lymphoma-expressed FasL, and demonstrated enhanced T cell persistence and anti-tumor activity. Similarly, Ren et al. applied CRISPR/Cas9 gene-editing to ablate Fas expression by CD19-targeting CAR T cells, resulting in reduced activation-induced T cell death following exposure to CD19+K562 CML tumor cells. However, the major issue with both these strategies is the risk associated with permanently eliminating Fas/FasL signaling in T cells, particularly given the crucial role FasL plays in maintaining T cell homeostasis. An alternate to ablation is the use of a non-signaling dominant negative receptor (DNR), which acts as a sink for FasL. Indeed, Yamamoto and colleagues explored such a strategy and reported improved persistence and effector function of TCR- and CAR-modified T cells in FasL+ murine melanoma and human xenograft B-ALL models, respectively. Oda et al extended upon this approach by developing a chimeric “switch” receptor with the incorporation of the 41BB endodomain, thereby allowing T cells to harness and invert the apoptotic FasL signal to one that was costimulatory. This produced pro-survival signaling and metabolic changes in T cells, resulting in enhanced proliferation and anti-tumor activity in leukemia and pancreatic cancer models. While these strategies have proven effective in augmenting T cell activity in the TME, each was designed to benefit only the engineered cells and confer no protective advantage to adjacent cells with effector potential. In contrast, our secreted decoy was designed to not only provide autocrine protection but also paracrine protection (as illustrated in our CFPAC-1 xenograft model), thereby potentiating the benefit of our strategy.

The TME is replete with cells including TILs, NK cells and NKT cells whose inherent effector potential is inhibited due to tumor-imposed immunosuppression or exhaustion. Such a phenomenon has been reported by several groups including Woroniecka et al, who documented high level expression of molecules synonymous with T cell exhaustion including PD-1, LAG-3, TIGIT, and CD39, as well as diminished production of effector cytokines such as IFN-γ, IL2, and TNFα by human glioblastoma-resident TILs vs matched peripheral blood T cells. Not surprisingly, therefore, TME modulation with recombinant immunostimulatory cytokines and checkpoint inhibitors has been explored as a means to rejuvenate their effector potential. For instance, Klapper et al. demonstrated that administration of high-dose IL-2, with resultant lymphocytosis, in metastatic renal cell carcinoma patients produced durable complete and partial responses. Similarly, the clinical use of checkpoint inhibitors to block tumor-expressed suppressive molecules like PD-1 and CTLA-4 has also been associated with clinical benefit. For instance, Tumeh et al. reported increased T cell infiltration and proliferation in metastatic melanoma patients treated with anti PD-1 therapy, which directly correlated with reduction in tumor size. While effective in producing clinical responses in patients with certain tumor types (e.g., metastatic melanoma, renal carcinoma, and non-small cell lung cancer), systemic therapies like high-dose IL-2 and checkpoint inhibitors often exhibit non-tumor tropic drug-related adverse effects including thrombocytopenia, liver toxicity, pneumonitis and other autoimmune phenomena that can result in patient mortality. Thus, considering the pivotal role that endogenous effectors can play in mediating anti-tumor responses, particularly if this benefit could be concentrated at the TME (thereby mitigating off target effects), we sought to design our decoy to be soluble in order to shield both engineered and bystander cells from FasL-mediated apoptosis. Furthermore, since the soluble decoy molecule is secreted only upon CAR engagement with its target on tumor cells, its benefit is concentrated in the TME. Finally, to also augment the proliferation and persistence of both engineered and bystander cells we linked our decoy to IL-15 -a cytokine known to promote growth and maintenance of effectors like memory CD8+ T cells and NK cells. Indeed, the additive benefit of both shielding (from FasL) and supporting the proliferation of both engineered and bystander cells can be appreciated in our CFPAC1 model, where we observed superior anti-tumor activity with PSCA CAR T cells engineered with F+15 administered in combination with CAR.MUC1 T cells.

While the current study focused on the Fas-FasL axis using CAR engineered cells directed to pancreatic cancer-expressed TAAs, our platform can be customized for different disease types and extended to virtually any suppressive molecule at the TME (e.g., PD-L1, TGFβ, and IL-10). Furthermore, our decoy is compatible with various stimulatory signals including cytokines (e.g., IL-7, IL-21) as well as other co-stimulatory ligands such as 4-1BBL and CD70. Additionally, as all adoptively transferred cellular therapies are prone to tumor-drive immunosuppression, our approach could be applied to strengthen the clinical efficacy of a range of cellular therapies beyond CAR T cells such as TIL, transgenic TCR T cell, and NK cell therapies. Finally, one of the pitfalls of adoptively transferred CAR- and TCR-modified T cells is that the engineered cells target a single TAA, leading to the risk of tumor immune escape as has been reported in the context of CAR-CD19-treated patients where approximately half of the relapses were due to loss of CD19. While testing this hypothesis was beyond the scope of this study, we speculate that incorporation of the decoy can indirectly mitigate this risk of tumor immune escape due to its ability to harness bystander effector cells with the potential to recognize a heterogeneous range of tumor-expressed target antigens beyond those recognized by the adoptively transferred cells.

In nature, the Fas-FasL axis is designed to maintain homeostasis of immune effectors such as antigen-specific T cells by limiting hyperactivation and uncontrolled expansion. Thus, any engineered approach to modulate this pathway requires careful consideration, given the risk of systemic toxicity as a result of autonomous T cell proliferation. Indeed, naturally occurring mutations of Fas have been described and shown to result in autoimmune lymphoproliferative syndrome (ALPS) characterized by uncontrolled lymphoproliferation leading to disorders including hemolytic anemia, thrombocytopenia, and neutropenia. Of note however, in our in vitro as well as in vivo assessment of the decoy-engineered T cells, we saw no evidence of autonomous T cell proliferation. Indeed, both T cell proliferation and persistence were dependent on the presence of target antigen and upon removal, as shown in our in vivo study (FIG. 5), T cells declined in number gradually with decreasing antigen availability as the tumor shrank. While these properties of Fas decoy secreting cells in our preclinical assessment indicates their safety profile, incorporation of a suicide switch (e.g., inducible caspases) to eliminate transgenic T cells in the event of unexpected toxicity could be considered during clinical translation.

To summarize, this study demonstrates the feasibility of engineering T cells to express a secreted Fas decoy receptor that can enhance T cell expansion/persistence and anti-tumor activity when targeting FasL+ tumor. The potency of T cells was further increased by incorporating IL-15 into the decoy, resulting in a Fas decoy-IL-15 fusion molecule, without adversely impacting T cell phenotype, antigen-specificity, and dependency. Importantly, our soluble decoy strategy exhibits bystander effects and thus, bears the potential to support survival as well as tumor-killing activity of endogenous T and other effector (e.g., NK, NKT, and 76) cells in the tumor periphery.

### Example 8: Useful Methods and Materials for Practicing Embodiments Herein

The methods, materials, compositions, and techniques described below in this Example provide examples for practicing embodiments herein. The examples are not limiting in that there may be other methods, materials, compositions, and/or techniques known to one of ordinary skill in the art that can be used to practice embodiments herein.

### Donors and Cell Lines

Peripheral blood mononuclear cells (PBMCs) were obtained from healthy volunteers after informed consent on a protocol approved by the Baylor College of Medicine Institutional Review Board. CAPAN1, CFPAC1, K562, and 293T cell lines were obtained from the American Type Culture Collection (Rockville, MD) and were grown in complete IMDM medium—Iscove's Modified Dulbecco's Medium (IMDM, Gibco BRL Life Technologies, Inc., Gaithersburg, MD) supplemented with 10% heat-inactivated fetal bovine serum (FBS) (Hyclone, Waltham, MA) and 2 mM L-GlutaMAX (Gibco BRL Life Technologies, Inc., Gaithersburg, MD). All cell lines were maintained in a humidified incubator containing 5% carbon dioxide (CO₂) at 37° C.

### Generation of Retroviral Constructs and Retroviral Supernatant

A human, codon-optimized CAR was synthesized with specificity against PSCA using the published scFv sequences (Leyton et al, Clinical Cancer Research, 748-96; 2008 and Leyton et al, Protein Engineering, Design and Selection, 209-16; 2009), which was cloned in-frame with the IgG2-CH3 domain (spacer), CD28 co-stimulatory domain, and the zeta (ζ) chain of the T cell receptor (TCR) CD3 complex in an SFG retroviral backbone to make a 2^{nd} generation CAR. A 2^{nd} generation MUC1-specific CAR containing 41BB as a costimulatory domain was constructed as described previously (Bajgain et al., Journal for ImmunoTherapy of Cancer (2018) 6:34). To generate the Fas decoy (FD) vector, using DNA 2.0 (Menlo Park, CA), a codon-optimized sequence was designed encoding the signal peptide and extracellular domain of the human Fas (CD95) protein with the restriction sites Xho1 and Sph1 incorporated up and downstream, respectively (IDT DNA Technologies, Coralville, IA). The FD DNA insert was incorporated into an SFG retroviral vector that contained the fluorescent marker mOrange linked by an IRES sequence downstream of the insertion site. To generate the FD+15 construct, a codon-optimized sequence encoding IL-15 without the signal peptide was linked to the C-terminus of the FD sequence using a G-S(GGGSGGGGSGGGGSGGG; SEQ ID NO:1) linker. Similarly, to generate the FΔ+15 vector in which the IL-15 component was functional but the decoy was defective through the incorporation of an amino acid substitution (R86S) mutation that was previously published to prevent interaction with FasL. Constructs encoding PSCA antigen, click beetle green luciferase CBG99, and a previously described firefly luciferase mutant Akaluc were generated by incorporating synthesized linear DNA fragments (IDT DNA Technologies, Coralville, IA) encoding these proteins into an SFG retroviral vector backbone upstream of GFP tag linked by an IRES sequence. Retroviral supernatant for all constructs was generated by transfection of 293T cells, as described previously (ref). Briefly, 3.5×10⁶ 293T cells were plated in 100 mm tissue culture treated dishes (BD Biosciences, Bedford, MA) in 10 mL complete IMDM medium. A day later, cells were transfected with PegPam, RDF, and the DNA construct loaded into GeneJuice Transfection Reagent (Millipore, Burlington, MA). Retroviral supernatant for GFP-Firefly luciferase (GFP_FFLuc) vector was obtained from a previously described producer cell line(ref). Supernatant from the transfected cells was collected at 48 and 72 hrs, pooled together, sterile filtered using 0.2 μm filters (Pall Corporation, Port Washington, NY) and stored at −80° C. until transduction.

Generation of CAR T cells

To generate CAR T cells, 1×10⁶ PBMCs were plated in each well of a non-tissue culture-treated 24-well plate that had been pre-coated with OKT3 -an anti-CD3 antibody (1 mg/mL) (Ortho Biotech, Inc., Bridgewater, NJ) and CD28 (1 mg/mL) (Becton Dickinson & Co., Mountain View, CA). Cells were cultured in complete media (RPMI-1640 containing 45% Clicks medium (Irvine Scientific, Inc., Santa Ana, CA), 10% FBS and 2 mM L-GlutaMAX), which was supplemented with recombinant human IL-2 (50 U/mL, National Institutes of Health, Bethesda, MD) on day 1. On day 3 post OKT3/CD28 T blast generation, 1 mL of retroviral supernatant was added to a 24-well non-tissue culture-treated plate pre-coated with recombinant fibronectin fragment (FN CH-296; Retronectin; Takara Shuzo, Otsu, Japan) and centrifuged at 2000G for 90 minutes. OKT3/CD28 activated T cells (0.2×10⁶/mL) were resuspended in complete media supplemented with IL-2 (100 U/mL) and then added to the wells and centrifuged at 400G for 5 minutes. To generate CAR and FD, FΔ+15 or FD+15-expressing cells, activated T cells were transduced sequentially, first with the CAR construct (on day 3) and then with either FD, FΔ+15, or FD+15 on day 4, respectively. T cells used in the in the in vitro experiments involving live cell bioluminescence imaging or in vivo experiments underwent a third transduction on day 5 with either CBG99, Akaluc, or GFP-FFLuc retroviral supernatant. Transduction efficiency was measured 3 days after the last transduction by flow cytometry.

### CAPAN1 Transduction

CAPAN1 cell lines that expressed transgenic PSCA were generated to ensure homogeneous antigen expression. To do this, PSCA-GFP retroviral supernatant was plated in a non-tissue culture-treated 24-well plate (1 mL/well), which was pre-coated with a recombinant fibronectin fragment. CAPAN1 cells (0.2×10⁶ per well) were added to the plates and then transferred to a 37° C., 5% CO₂ incubator. Transgene expression was analyzed by flow cytometry 1 week post-transduction. Cells were subsequently sorted based on GFP expression using a MoFlo flow cytometer (Cytomation, Fort Collins, CO).

### Flow Cytometry

The following antibodies were used for T cell phenotyping: CD3-APC, CD4-Krome Orange, CD8-Pacific Blue, CD69-ECD, CD45RO (Beckman Coulter Inc. Brea, CA), CCR7-FITC, CD25-FITC, CD28-PC7, CD95-PC5.5 (BD Biosciences, San Jose, CA). PSCA antigen and FasL expression on tumor cells was measured using anti-PSCA (Santa Cruz Biotechnology. Inc., Dallas, TX) and anti-human CD178-APC (BD Biosciences, San Jose, CA), respectively. CAR molecules were detected using Goat anti-human F(ab′)2 antibody conjugated with AlexaFluor647 (109-606-097) (Jackson ImmunoResearch Laboratories, Inc., West Grove, PA). Cells were stained with saturating amounts of antibody (˜5 μL) for 20 min at 4° C., washed (PBS, Sigma-Alrich, St. Louis, MO), and then acquired on Gallios™ Flow Cytometer (Beckman Coulter Inc., Brea, CA). Analysis was performed using Kaluza® Flow Analysis Software (Beckman Coulter Inc.).

### ⁵¹Chromium-Release Assay

The cytotoxicity and specificity of engineered T cells was evaluated in a standard 4-6 hr ⁵¹Cr-release assay, using E:T ratios of 40:1, 20:1, 10:1, and 5:1. Effector T cells were co-incubated in triplicate with target cells labeled with ⁵¹Cr in a V-bottomed 96-well plate. At the end of the incubation period at 37° C. and 5% CO₂, supernatants were harvested, and radioactivity counted in a gamma counter. The percentage of specific lysis was calculated as follows: % specific cytotoxicity=[experimental release (cpm)-spontaneous release (cpm)]/[maximum release (cpm)-spontaneous release (cpm)]×100.

### Cell Viability Assay

Recombinant Fas ligand (BioLegend, San Diego, CA) (200 ng/mL) was added to CAR T cells maintained in fresh T cell medium or conditioned medium obtained from activated cultures of CAR-only or CAR.FD T cells. After overnight incubation, cells were labeled with Annexin V-APC and 7AAD according to manufacturer's protocol and cell viability was monitored using a Gallios™ flow cytometer.

### T Cell Stimulation Assay

To measure T cell expansion upon antigen stimulation, 1×10⁶ T cells were cultured with 1×10⁶ irradiated K562 or CAPAN1 tumor cells overexpressing PSCA in the absence of exogenous cytokines. Tumor cells were irradiated (100Gy) to halt their expansion using Rad Source RS2000 Biological X-Ray Irradiator (Rad Source Technologies, Buford, GA) before placing them in culture with T cells.

### Measurement of Decoy and Cytokine Production

Transgenic Fas-decoy production was measured in supernatant harvested from activated cultures using a soluble Fas ELISA kit (R&D Systems, Minneapolis, MN) according to manufacturer's instructions. To measure the production of the FΔ+15 and FD+15 fusion molecules, identical sample replicates were used in soluble Fas and IL-15 ELISA assays. To measure the production of effector cytokines by T cells, supernatant harvested at 48 hrs post-activation was used to perform a 13-plex multiplex assay (Millipore Sigma, Danvers, MA) according to manufacturer's instructions.

### Co-Culture Experiments

T cell:tumor co-culture experiments were performed to assess the in vitro anti-tumor activity of T cells using CFPAC1 (0.1×10⁶ cells) or CAPAN1 (0.25×10⁶ cells) tumor cells overexpressing PSCA as targets. Tumor cells were plated in a 6-well tissue culture treated plates in 3 mL of culture medium. A day later, 5×10⁴ CAR, CAR.FD, CAR.FΔ+15, or CAR.FD+15-modified T cells were added to tumor cells. Anti-tumor activity was monitored on days 3 and 6 using flow cytometer to quantify cells. All cells in the wells were collected, labeled with a CD3-APC antibody, mixed with CountBright™ Absolute Counting Beads (C36950; Invitrogen, Eugene, OR), and 7-AAD was added to exclude dead cells. Acquisition was halted at 2500 beads.

### In Vivo Studies

To assess the in vivo activity of decoy-engineered T cells, 6-to-8 week old female NSG mice (NOD.Cg-Prkdcscid IL-2rgtm1Wjl/SzJ, Jackson ImmunoResearch Laboratories, Inc., West Grove, PA) were injected with 5×10⁶ PSCA-overexpressing CAPAN1 cells suspended in 1×PBS subcutaneously (s.c.) into the left flank. Once the tumor reached a size of approx. 140 mm³ (˜4 weeks), animals were injected intravenously (i.v.) with 3×10⁶ GFP-FFLuc+ CAR, CAR.FD, CAR.FΔ+15 or CAR.FD+15 T cells. Tumor volume was monitored by caliper measurement and calculated using the formula: length ×width ×width/2. T cell expansion and persistence was monitored using the IVIS Lumina In vivo Imaging system (Caliper Life Sciences, Hopkinton, MA) 10 minutes after injection (i.p.) with 100 μL of D-luciferin (15 mg/mL) and the images were analyzed using Living Image software (Caliper Life Sciences, Hopkinton, MA). Mice were euthanized once the tumor volume reached the protocol limit (1500 mm³) or in the event of tumor ulcerations that grew >2 mm in diameter despite treatment or recurred. To assess the bystander effects of the decoy-producing T cells, 6-to-8 week old female NSG mice (NOD.Cg-Prkdcscid IL-2rgtm1Wjl/SzJ, Jackson ImmunoResearch Laboratories, Inc., West Grove, PA) were injected with 5×10⁶ wildtype CFPAC1 cells suspended in 1×PBS s.c. into the left flank. Once the tumor reached a size of approximately 140 mm³ (-4 weeks), animals were injected intravenously with 2×10⁶ CBG99+ CAR MUC1 T cells together with 2×10⁶ Akaluc+ CAR PSCA, or CAR.FD, or CAR.FD+15 T cells. Tumor size was measured by weekly caliper measurement and tumor volume (mm³) was calculated by calipers (length ×width ×width/2). T cell expansion and persistence was monitored using the IVIS Lumina In vivo Imaging 10 minutes after intraperitoneal injection with 100 μL of D-luciferin (15 mg/mL) to capture luminescence produced by the bystander (CAR MUC1) T cells. Four hours later, imaging was repeated with an i.p. injection of 100 μL solution of 5 mM Akalumine-HCL (Tokeoni) (Millipore Sigma, St. Louis, MO) to capture luminescence of CAR PSCA, CAR.FD, or CAR.FD+15 T cells. All in vivo analysis was performed using Living Image software (Caliper Life Sciences, Inc., Hopkinton, MA). Experiments were performed according to Baylor College of Medicine Animal Husbandry guidelines.

### Statistical Analysis

Results are reported as mean±SEM unless stated otherwise. All statistical analyses were performed using GraphPad Prism software. Statistical significance between/among groups was determined using one-way ANOVA, two-way ANOVA, or unpaired two-tailed t tests. P-values less than 0.05 were considered statistically significant.

### Example 9: Description of Cancer Immunotherapies

### Cancer Immunotherapy

For many years, the cornerstones of cancer treatment have been surgery, chemotherapy, and radiation. Although these approaches have contributed to improved outcomes, most malignancies still carry a poor prognosis.

To increase the probability of success, current research has focused on targeted anticancer approaches that can provide individualized therapy to combat the complexity of most malignancies. These approaches rely on both the identification of different immune effector cells and an understanding of how they exert their effector functions. Researchers have exploited this knowledge to develop immunotherapeutic approaches that can be grouped into three main categories—vaccines, antibodies, and cell based therapies.

### Cancer Vaccine

Cancer vaccines utilize immunogens to elicit endogenous immune responses against the tumor. A wide range of cancer vaccines have been preclinically and clinically explored including malignant cells or their components (e.g. whole cell lysate, recombinant protein/peptide), as well as DNA plasmids and/or viral vectors encoding tumor antigens (with or without adjuvants) as well as DC-based vaccines.²²⁻²⁴ A number of these have advanced to late stage clinical trials, including the whole cell-based vaccine GVAX, which has been explored for the treatment of a range of solid tumor indications including prostate cancer, non-small cell lung cancer (NSCLC), and pancreatic cancer.

To date, the only Food and Drug Administration (FDA)-approved vaccine is sipuleucel-T (Provenge®)—an antigen-loaded [fusion protein consisting of prostatic acid phosphatase (PAP) and granulocyte-macrophage colony-stimulating factor (GM-CSF)] DC vaccine for the treatment of advanced prostate cancer. While vaccines have demonstrated therapeutic potential, clinical responses have been limited. Vaccines rely on activating endogenous immune effector cells, which are often anergized/tolerized due to prolonged exposure to the immunosuppressive tumor milieu and/or scarce in heavily pre-treated patients with advanced diseases.

### Antibodies

Antibodies have been used as immunotherapeutic agents to treat cancer for over 2 decades. They induce death of tumor cells by various pathways including (i) antibody-dependent cellular cytotoxicity (ADCC), where antibody-target antigen engagement induces cell death, (ii) inhibition of signaling pathways essential for tumor survival and progression, and (iii) complement/Fc-receptor-expressing immune cell activation to initiate anti-tumor responses.^{29,30}

The FDA approval of rituximab (Rituxan)—a monoclonal antibody that binds to CD20 -a tumor antigen expressed in various B cell malignancies—has revolutionized the treatment of CD20+ leukemia and lymphoma. Upon engagement with CD20, rituximab induces ADCC, complement-dependent cytotoxicity (CMC), and apoptosis by disrupting signaling pathways associated with cell survival such as p38 MAPK, NF-κB, ERK ½, and AKT anti-apoptotic signaling cascades³¹. Similarly, trastuzumab (targeting HER-2) which causes tumor lysis by ADCC and disrupts MAPK and PI3/Akt cell survival pathways, has been utilized to treat HER-2+ breast and gastric cancer.³⁴⁻³⁶

Apart from directly targeting cancer cells, antibodies can also be deployed to perturb the engagement of tumor-expressed inhibitory ligands (often known as checkpoint molecules) and their receptors on immune cells. For instance, ipilimumab (Yervoy) targeting CTLA-4 and pembrolizumab/nivolumab (Keytruda/Opdivo) targeting PD-1 have been approved for the treatment of various types of cancers including melanoma, renal cell carcinoma, colorectal cancer, NSCLC, head and neck cancer, and cervical cancer, with ongoing trials exploring the extension to other malignancies either as monotherapies or in combination with other modalities.³⁷⁻⁴⁰

However, due to their short half-life (2-3 weeks on average), repeated administrations are often necessary to achieve durable anti-tumor benefits, which increases the associated costs.⁴⁴ Additionally, penetration across barriers such as the blood-brain barrier and dense tumor stroma in certain solid tumors (e.g. pancreatic cancer) limit the clinical benefit in some malignancies.⁴⁵

### Cell Based Therapies

Cell-based therapy involves modification of autologous or allogeneic immune cells to fight a range of medical conditions. Since cell based approaches utilize the natural effector functions of endogenous immune cells, such therapies minimize off target toxicities and provide durable long term responses.

### T Cell Therapy

Adoptive T cell transfer involves the ex vivo preparation and administration of T cells to patients to treat diseases. Rosenberg and colleagues at the National Cancer Institute (NCI) isolated tumor-infiltrating lymphocytes (TILs) from resected melanoma lesions, which were non-specifically expanded ex vivo using IL-2 and allogeneic lymphocytes as feeders.^{50, 51} When administered to patients with metastatic melanoma at cell doses ranging from 1.1-16×10¹⁰ (average 6.3×10¹⁰ cells/patient) in combination with high-dose IL-2 (720,000 IU/kg every 8 hours) following lymphodepleting chemotherapy, these TILs produced objective (51% response rate) and durable benefit (2 to >30 months). Though associated with clinical benefit, the need for tumor material limits the spectrum of tumors for which this therapy can be applied.

To overcome this limitation, some groups have explored approaches to isolate and selectively expand tumor reactive T cells from patient peripheral blood. Ex-vivo expanded EBV-specific T cells [generated using patient-derived PBMCs co-cultured with autologous EBV-transformed lymphoblastoid cell lines (EBV-LCL)] for the treatment of EBV-associated Hodgkin and non-Hodgkin lymphoma (HL/NHL) demonstrated complete responses (CRs) in 52.4% (11 of 21) patients with relapsed/resistant disease and event-free survival (EFS) in 82% of the patients in remission with high-risk or multiply relapsed disease.^{53, 54}

More recently, strategies have been developed to expand T cells directed against non-viral TAAs including PRAME, SSX2, MAGEA4, NY-ESO-1, and Survivin using DCs loaded with overlapping peptide libraries (15mers overlapping by 11aa) spanning each of the target antigens. Phase I/II clinical trials using these cells to treat both hematologic (lymphoma, leukemia, myeloma) and solid tumors (breast, pancreatic cancer) are currently ongoing.

Thus, targeting tumors using ex vivo prepared T cell populations whose antigenic specificity is mediated via the native T cell receptor (TCR) is feasible, but manufacturing is complex, requiring repeated antigenic stimulations in open culture systems. The lead time for manufacturing antigen specific cell lines can range from 4-16 weeks. Generating a sufficient number of tumor-reactive T cells ex vivo is challenging especially as evidence suggests that prolonged culture time can result in inferior anti-tumor responses when transferred back to the patient.

### Genetically Engineered T Cells

Genetic engineering can be used to confer T cells with a variety of characteristics including the ability to target and kill malignant cells through the transgenic expression of tumor-targeted receptors. To date two approaches have been tested clinically—transgenic expression of (i) tumor-targeted peptide-specific αβ TCRs or (ii) chimeric antigen receptors (CARs), which are synthetic receptors that combine the antigen recognition properties of a monoclonal antibody with the signaling capacity of a TCR.

### Transgenic αβ TCR-Modified T Cells

Transgenic expression of genes encoding tumor-targeted αβTCRs isolated from TILs has been explored as a means of genetically conferring T cells with the ability to recognize malignant cells. The feasibility of this approach was first tested in the clinic by Rosenberg and colleagues at the NCI who isolated an HLA-A2-restricted MART-1-specific TCR (called DMF4) from a TIL clone obtained from a patient with metastatic melanoma who achieved near complete tumor regression post-TIL therapy.⁵⁵ Subsequently, this group engineered autologous T cells from HLA-A2+ individuals with the same transgenic TCR and among 31 patients treated with cell doses ranging from 1 to 86×10⁹ there were no reported toxicities and 4 patients (13%) achieved durable tumor regressions (lasting >20 months).⁵⁶ While this first in human experience demonstrated the feasibility and safety of MART1-targeted αβTCR therapy, the clinical effects were underwhelming, prompting investigation into strategies to enhance potency.

T cell activation and function primarily depends on the ability of the TCR to interact with the peptide-MHC (pMHC) complex and the higher the TCR affinity the more potent the consequent activation. Thus, the group at the NCI screened multiple T cell clones (using an IFNg ELISA-based system) to identify a higher affinity TCR [10,865 μg/mL IFNg vs 2,397 μg/mL; high affinity TCR (DMF5) vs DMF4]. In the subsequent clinical trial infusion of DFM5 TCR-modified T cells at doses ranging from 1.5-107×10⁹ cells produced objective responses in 30% of the 20 infused patients. However, the increased efficacy was achieved with an increase in ‘possibly related’ toxicities associated with transgenic T cell recognition of MART1 expressed on non-malignant tissues. The clinical consequences included anterior uveitis, vitiligo and ototoxicity resulting in hearing loss, which were detected in 42% of the patients infused and were managed by local administration of steroids.⁵⁷

However, the substantial clinical benefit achieved using the MART-1 TCR-modified T cells prompted extension to a wide-range of malignancies using a spectrum of TCRs. As highlighted by the DMF5 MART-1 TCR study, clinical efficacy is tightly linked with TCR affinity, and in nature, many naturally occurring TCRs targeting overexpressed self-antigens are low affinity due to thymic selection. Thus, to overcome this limitation, a number of investigators have explored strategies to affinity enhance TCRs by substituting one or two amino acids in the complementarity determining region (CDR) to enhance peptide-MHC engagement.⁵⁹ One of the first examples of the clinical use of such affinity-enhanced TCRs was a clinical trial conducted by Robbins and colleagues at the NCI who administered T cells modified with an HLA-A2-restricted NY-ESO-1-specific TCR [clone: 1G4 with 2 amino acid substitutions (threonine®leucine and serine®tyrosine] at cell doses ranging from 16-120×10⁹ in combination with systemic IL-2 (720,000 IU/kg every 8 hours) for the treatment of metastatic melanoma or metastatic synovial cell sarcoma. Overall, 11 of 20 (55%) patients with melanoma and 11 of 18 (61%) with synovial cell sarcoma achieved durable objective clinical responses (based on RECIST criteria) with complete regressions documented in 35% (n=7) of melanoma patients and 6% (n=1) of synovial cell sarcoma patients.⁶⁰ Of note, though, infused patients experienced toxicities related to the preconditioning regimen and high dose IL-2, including transient neutropenia and thrombocytopenia, no toxicities were attributed to the T cell infusions.⁶⁰ Reported toxicities included transient neutropenia, pancytopenia, dehydration, hypotension, and hyponatremia. Additionally, 3 patients developed skin rashes (grade 3 or lower) and 3 patients developed GVHD, which manifested as colon inflammation and diarrhea and resolved either spontaneously or upon administration of steroids.⁶¹

The examples show that while affinity-enhanced TCRs have proven clinically effective, trials of this approach have not always proven safe. Subsequent investigations demonstrated that the modifications made during affinity enhancement rendered the transgenic TCR specific for an epitope of titin—a striated muscle-specific protein expressed at high levels in cardiomyocytes. Indeed, autopsy results of these patients revealed substantial infiltration of the engineered MAGE-A3-specific T cells into the heart tissue and significant cardiac myonecrosis.⁶³ Thus, the clinical use of affinity-enhanced TCRs can provide significant clinical benefit to patients with metastatic disease but modification of the native TCR binding may also result in erroneous recognition of non-targeted self-antigens and cause significant toxicities and even death.

Apart from the “off tumor” effects of transgenic TCRs, there is the potential for cross-pairing between the transgenic and native TCR producing an autoreactive specificity. This has not been reported clinically but in a preclinical study Bendle and colleagues reported severe GVHD and death of mice treated with OT-1 TCR T cells, which was attributed to the mispairing of endogenous and transgenic TCR chains.⁶⁴

However, perhaps the major limitation of transgenic TCR therapy is the limited specificity of the infused product, which recognize a single HLA-restricted epitope of a single tumor associated antigen, precluding broad implementation of this approach. Furthermore, tumor cells are known to downregulate MHC class I molecules and modulate antigen/epitope expression as an immune evasion mechanism⁶⁹, making alternative forms of cell-based therapies that can bypass restricted TCR-MHC mediated recognition of tumor antigens an attractive alternative.

### CAR T Cells

One approach to cancer immunotherapy entails genetically engineering a patient's T cells to express chimeric antigen receptors (CARs) that recognize and attack tumor cells. The CAR consists of an antibody or ligand-derived targeting ectodomain fused with a hinge, a transmembrane domain, and intracellular T cell signaling domains. When expressed by a T cell, CARs confer antigen specificity determined by the targeting domain. In contrast to conventional T cell receptors (TCRs), which recognize antigens in a major histocompatibility complex (MHC)-dependent manner, CARs can potentially redirect the effector functions of a T cell toward any protein or non-protein target expressed on the cell surface. This strategy thereby avoids the requirement of antigen processing and presentation by the target cell and is applicable to non-classical T cell targets like carbohydrates. Circumventing human MHC-restriction renders the CAR T cell approach as a universal treatment, broadening the potential applicability of adoptive T cell therapy.

The earliest version (so-called 1^{st} generation CARs) simply combined a tumor-specific scFv with the CD3ζ chain. Although preclinical studies were promising, the results from early phase I clinical trials in patients with diseases including renal cell carcinoma, ovarian cancer, and neuroblastoma were underwhelming, which was primarily attributed to limited in vivo T cell expansion and persistence. Early clinical studies have established that CAR-engineered T cells could be safely administered to patients, but the lack of clinical responses emphasized the need for further improvement in CAR design to achieve durable anti-tumor responses.

Consequently, several groups started exploring strategies to enhance the persistence of T cells with the hypothesis that signal 1 alone (transmitted via the CD3ζchain) was insufficient to drive sustained T cell activity in vivo. This led to the development of 2^{nd} and 3^{rd} generation CARs containing two or three co-stimulatory endodomains respectively, including CD28, 41BB, and OX40 to promote crucial cellular functions such as proliferation, cytokine production, and survival.^{75, 76} And indeed, when clinically implemented substantial success has been achieved.

Grupp et al reported complete remissions in 2 pediatric patients with relapsed/refractory pre-β-cell ALL following administration of 2G.CD19.41BB CAR T cells (cell dose: 0.14-1.2×10⁷/kg).⁷⁸ This study was later expanded to treat 30 B-ALL patients (25 pediatric and 5 adults), with 90% of patients achieving complete responses, which correlated with significant in vivo T cell expansion and persistence for up to two years post-infusion in some patients.⁷⁹ To date, CD19 CAR T cells have been tested in clinical trials in patients with B cell tumors at various institutions (such as the NCI, MSKCC, CHOP, UPenn, Baylor College of Medicine and FHCRC) and has consistently demonstrated remarkable initial clinical response rates ranging from 70->90%.

Though initial results of 2G.CAR CD19 T cells based on short term follow-up was extremely impressive, durability has emerged as a major issue. For instance, in the relapsed/refractory B-ALL patients treated at UPenn, the initial complete remission rate was 93% when patients were assessed 1 month post-infusion. However, at the 1 year assessment the response rate had declined to 55%.⁸⁰ Similarly, FHCRC reported that 31% (9 of 29) of adult B-ALL patients relapsed during long-term follow up, despite initial bone marrow remission rates of 93% and the majority of relapses were ascribed to immune escape with the emergence of CD19 negative tumors.⁸⁰

Other significant drawbacks of adoptively transferring 2G.CD19 CAR T cells include “on target off tumor” effects resulting in B cell aplasia as well as cytokine release syndrome (CRS) and neurotoxicity frequently documented in infused patients and associated with rapid in vivo expansion and tumor lysis mediated by the infused cells.^{82,83} This leads to the overproduction of inflammatory cytokines such as IL-6, IFNg, and IL-1, which trigger a variety of complications including fever, fatigue, hypotension, vascular leakage, and multi-organ system failure.^{84, 85}

Approaches to address some of these limitations of CD19 CAR T cell therapy are currently being investigated by several groups. For instance, targeting multiple TAAs simultaneously to prevent CD19 negative immune escape/frequency of relapses (e.g. PLAT-05 trial targeting CD19 and CD22 in pediatric ALL), combination therapy using CD19 CAR T cells in combination with checkpoint inhibitors (e.g. pembrolizumab) to enhance the efficacy of infused CAR T cells⁸⁷(ASCO abstract, 2017) and blocking antibodies against mediators of CRS-associated toxicities such as tocilizumab (anti-IL-6 receptor antibody)^{82,83,88}.

Overall, CD19 CAR T cell therapy's outstanding remission rates in certain B cell malignancies have established CAR T cells as one of the most successful cancer immunotherapies and led to the FDA approval of two CD19 CAR T cell therapy products-Kymirah (tisagenlecleucel) for the treatment of pediatric ALL and Yescarta (axicabtagene clioleucel) for the treatment of adult relapsed/refractory B cell lymphoma.⁸⁹

The clinical responses achieved using CAR T cells for hematologic malignancies inevitably prompted the extension of this approach to solid tumors including breast cancer (targeting CEA, mesothelin), neuroblastoma (targeting GD2), glioma (targeting EGFRvIII), mesothelioma [targeting mesothelin (MSLN), FAP], pancreatic cancer (targeting MSLN), lung cancer (targeting GPC3, MSLN), and sarcoma (HER2).^{3, 90-95} However, durable and complete clinical responses have been rare. For example, Katz and colleagues administered 2G.CEA.28 CAR T cells to 6 patients with adenocarcinoma liver metastases. Of these one had stable disease for up to 102 weeks post-infusion while the rest succumbed to disease progression.⁹⁶ So, while the feasibility of CAR T cell therapy for solid tumors has been established with these clinical trials, there is a clear need for additional work in order to improve the efficacy profile.

### Challenges in Treating Solid Tumors

There are many features that discriminate solid tumors and hematologic malignancies and may limit the effectiveness of cell based therapies. The tumor stroma is composed of stromal cells such as cancer associated fibroblasts (CAFs) that secrete extracellular matrix proteins such as collagen and hyaluronan that act as a physical barrier limiting accessibility to malignant cells. Furthermore, solid tumors recruit or polarize accessory cells such as MDSCs, regulatory T cells, and M2 macrophages in order to directly suppress effector immune cells by either contact-mediated inhibition (i.e. upregulation of cell surface ligands such as PD-L1, B7-H4, and FasL), or secretion of inhibitory cytokines such as TGFβ, IL-4, IL-10, and IL-13.

### Tumor Stroma

Solid tumors that have grown beyond few cubic millimeters need to induce tumor angiogenesis, to receive nutrients, oxygen, and glucose for their high energy demand and growth. Tumor angiogenesis entails the development of new blood vessels from established vascular beds.

Pathological angiogenesis is mainly driven by an imbalance between pro-angiogenic and antiangiogenic signaling in the Tumor Microenvironment (TME). Key pro-angiogenic factors include, but are not limited to, VEGF-A, basic fibroblast growth factor (bFGF) and interleukin (IL)-8. These cytokines become ubiquitously abundant in the TME and overwhelm angiostatic signals, such as angiostatin and endostatin, thereby inducing a pro-angiogenic switch. In fact, not only do cancer cells secrete high amounts of VEGF and can contribute to VEGF-independent angiogenesis (by liberating various pro-angiogenic molecules, such as placental growth factor (PlGF), VEGF-C, VEGF-D, and platelet-derived growth factor (PDGF)-C) but they can also respond in an autocrine or paracrine manner to prosurvival and prometastatic VEGF signaling. Although tumor angiogenesis is meant to support blood supply to the tumor, the resulting vessel network is leaky, chaotically organized, immature, thin-walled, and ill-perfused.

This generates a hypoxic (i.e., less oxygenated) and acidic (due to increased anaerobic glycolysis of cancer cells) TME that facilitates the selection of cancer cells with genetic (i.e., enumeration of mutations favoring malignancy) and epigenetic alterations that enhance their aggressiveness. Importantly, hypoxia and acidosis facilitate attraction/development of immunosuppressive immune cells, reduce the cytotoxic activity of tumor-infiltrating effector T cells, and hamper delivery of chemotherapeutics and immunotherapeutic entities, as well as cancer cell killing in response to radio/chemotherapy and immunotherapy. This unproductive, highly aberrant angiogenesis contributes to maintain the protumorigenic and immunosuppressive TME and profoundly influences how cancer cells escape the anticancer immunosurveillance, metastasize, and respond to immunotherapy.

### Tumor Trafficking and Infiltration

Insufficient trafficking of immune cells to the tumor site represents another barrier for cell based therapies. Studies have shown that improved migration ability of infused immune cells to tumor sites may increase their antitumor immune response, and efficiency of adoptively transferred T cells infiltrating the tumor site correlates with clinical responses in patients. Trafficking to the tumor site requires expression and binding of adhesion receptors on both T cells and the tumor endothelium lining. In addition, T cell chemokine receptors must match the chemokines secreted by tumors. Chemokine/receptor mismatch has been shown to account for insufficient tumor localization of T cells. Many human tumors either secrete low levels of chemokines or chemokines for which effector T cells lack receptors. Consequently, adoptively transferred immune cells may fail find malignant cells.

### Tumor Antigen Expression and Heterogeneity

Once at the tumor site, there are another array of challenges faced by immune cells. A primary challenge in developing cell based therapies is identifying a tumor antigen that can be targeted safely and effectively. Ideally, cell based therapies should target a tumor-restricted antigen to avoid the risk of “on-target/off-tumor” toxicity that may result in an immune reaction against healthy tissues, and at least two criteria should be considered. First, the proposed Tumor Associated Antigen (TAA) should be differentially expressed on tumor cells compared with essential normal tissue. The CAR T-cell response is highly specific and can potentially bind to antigens even at low expression levels in normal tissues. Second, the TAA should be broadly expressed on the majority of tumor cells, as the success of CAR T cell therapy is largely dependent on expression of antigens on tumor cells. However, the major hurdle in the successful implementation of cell based therapies for heterogeneous solid tumors is the potential for immune escape when targeting a single tumor antigen.

Similarly, preclinical studies have reported improved tumor control when targeting more than one tumor antigen simultaneously, suggesting bi- or multi-tumor antigen specific CAR T cell products as a promising solution to mitigate immune evasion due to heterogeneous antigen expression/downregulation by the tumor. While these measures have mitigated the issue of immune escape, durable responses are yet to be seen due to the hostile solid tumor microenvironment which restricts immune cell expansion and survival as reported in various studies.

### The Suppressive Solid Tumor Microenvironment

In contrast to certain blood cancers that have responded well to CAR T cell therapy, solid tumors not only lack conventional co-stimulatory molecules, which are expressed on malignant and normal B lymphocyte targets in hematological malignancies, but also have evolved mechanisms to actively suppress the immune system. A number of immunosuppressive pathways can limit the full potential of cell based therapies. Inhibitory immune receptors are often expressed on T cells following persistent tumor antigen encounter, and these include T-cell membrane protein-3 (TIM-3), lymphocyte-activation protein-3 (LAG-3), T cell Ig and ITIM domain (TIGIT), cytotoxic T lymphocyte-associated antigen 4 (CTLA-4), and programmed death-1 (PD-1). The upregulation of these receptors limit the persistence and activity of the antitumor response immune cells (36).

Tumors employ multiple tactics to evade or misdirect tumor-specific immune response. Many soluble factors that suppress antitumor immune responses have been identified in tissue extracts, serum, and ascites fluid of cancer patients. Tumor cells and macrophages express prostaglandin E2 (PGE2), a soluble factor derived from arachidonic acid and produced by inducible cyclo-oxygenase 2 enzyme (8,36) that exerts its immunosuppressive effect through subversion of CD8 differentiation, suppression of T cell proliferation, and inhibition of CD4 T cell helper functions (97). The PGE2/EP2/protein kinase A (PKA) signaling pathway mediates immunosuppression through PGE2 (98), which in combination with adenosine activates PKA and blocks TCR activation. A small peptide called the “regulatory subunit I anchoring disruptor” (RIAD) dampens the negative effects of PKA on TCR activation-a function that researchers leveraged to improve T cell function. Increased inflammatory activity is a hallmark of the tumor microenvironment and creates an abundance of reactive oxygen species (ROS) that substantially impair antitumor activity.

Many tumors produce transforming growth factor beta (TGFη), which inhibits T cell activation, proliferation, and cytotoxicity. Transforming Growth Factor beta (TGF-β) is a pleiotropic cytokine produced in large amounts within cancer microenvironments that will ultimately promote neoplastic progression, notably by suppressing the host's T-cell immunosurveillance. This effect is mostly due to the well-known inhibitory effect of TGF-β on T cell proliferation, activation, and effector functions. Moreover, TGF-β subverts T cell immunity by favoring regulatory T-cell differentiation, further reinforcing immunosuppression within tumor microenvironments.

An example of such inhibition is that mediated by FasL—a molecule expressed by a range of cells including tumor cells and tumor endothelium, polymorphonuclear myeloid derived suppressor cells (PMN-MDSCs), M2 macrophages, Tregs, and CAFs—and upon engagement with the Fas receptor (expressed by activated T cells), induces apoptotic cell death. In nature, Fas/FasL signaling plays a central role in maintaining immune cell homeostasis. However, tumors have evolved to exploit this pathway in order to evade immune-mediated elimination. Indeed, elevated levels of tumor- and serum-FasL have been detected clinically in various solid tumors including bladder, breast cervical, gastric, and pancreatic cancers, where detection has been correlated with disease progression, increased metastasis, and poor survival.¹⁶⁵ These tumor-derived suppressive signals adversely impact immune cell effector function by various mechanisms such as limiting expansion and persistence, driving exhaustion, and inducing immune cell death.

### Example 10: Suppressive Effect of Tumor Microenvironment

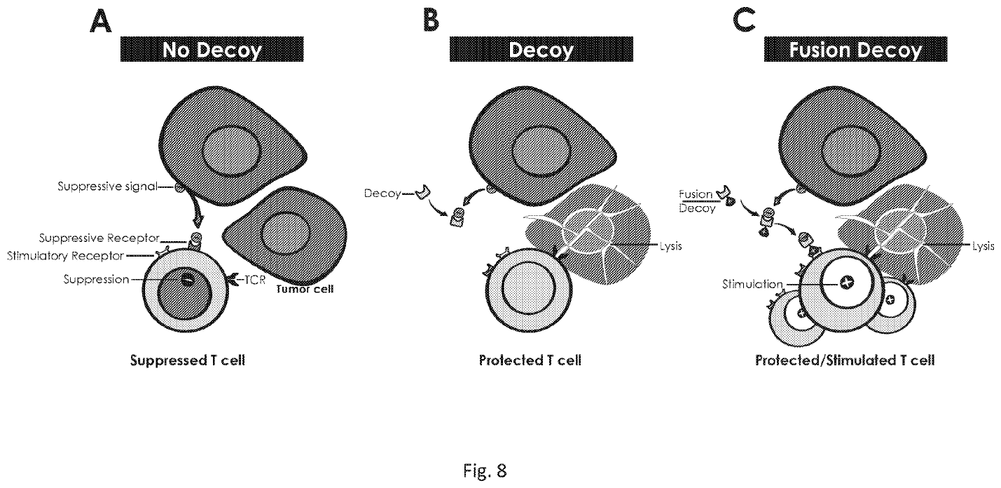

As shown in FIG. 8A, even when tumor-antigen-specific T cells are appropriately activated and home to tumor tissues, they must maintain their effector function and overcome local mechanisms of immune suppression in the tumor microenvironment in order for tumor eradication to be achieved. While we have the ability to effectively generate potent tumor-specific effector T cells through active immunization or adoptive T-cell transfer, cancer cells possess several strategies to successfully evade immune attack mediated by T cells. For instance, tumor cells can inhibit T-cell proliferation, cause dysfunction of T cells, and induce apoptosis of T cells through the secretion of tumor derived soluble factors.

Early experiments, dating back more than 30 years, provided a clue that tumors can alter functions of immune cells. In these experiments, supernatants of various tumor cells were found to suppress proliferation and/or cytotoxicity of normal human peripheral blood mononuclear cells (PBMC) in vitro. These observations from several independent laboratories were interpreted to mean that immunosuppressive soluble factors were produced by human tumors but not by control normal tissue cells.

Tumor cells can secrete a variety of inhibitory factors such as cytokines, retrovirus-like peptides, over-produced normal metabolites, and inducible nitric oxide synthases which are each mediated by distinct cellular mechanisms. Some of the inhibitory factors may be associated with the surface of tumor cells, while others are released into the tumor milieu.

The best described soluble factors that can be inhibitory for T-cell function are, IL-10, FAS-L and TGF-β. These cytokines can be produced by the tumor cells themselves or by non-tumor stromal cells. These factors have been shown to inhibit dendritic cell-mediated CD8+ T-cell priming in vitro, and its presence in the serum of patients appears to have negative prognostic import in certain cancers. Thus, in order to ensure durable anti-tumor response and survival of adoptively transferred T cells targeting solid tumors, incorporation of strategies to protect them from the inhibitory effects of the TME are essential.

### Example 11: Strategies to Mitigate Immunosuppressive Effects of Tumor Microenvironment

Embodiments herein include novel strategies to intercept the immunosuppressive ligands using soluble decoy receptors with a stimulatory moiety in order to simultaneously protect T cells from tumor ligand mediated apoptosis as well as provide stimulatory cytokine signal to enhance their in vivo proliferation and persistence, as shown in FIGS. 8B and 8C. The soluble decoy receptor approach not only protects the transgenic cells but also neighboring, non-modified immune-effectors, thereby maximizing the potential of harnessing bystander benefit.

### Example 12: Suppressive Immune Ligand Defense Systems (SIELDS)

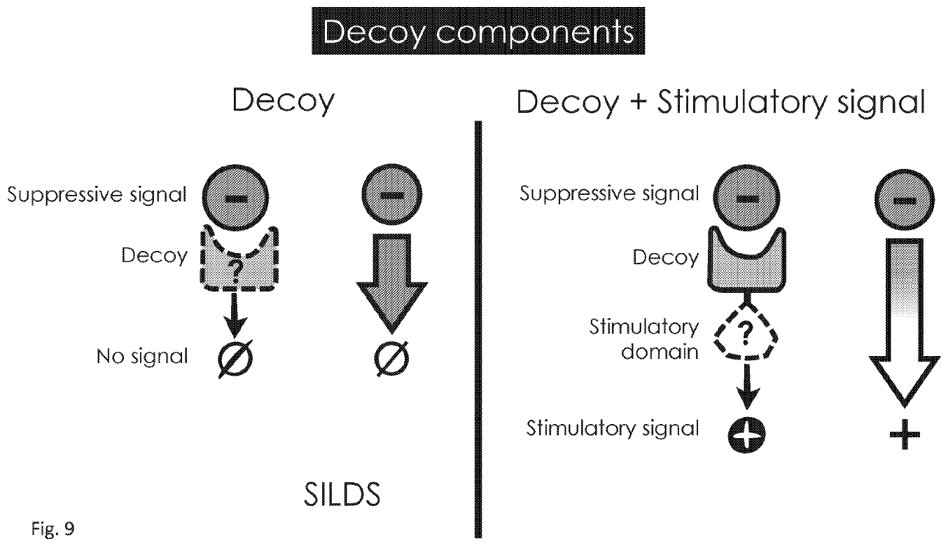

Embodiments herein include improvements of previous research around transgenically expressed soluble proteins. As shown in FIG. 9A, certain methods utilize soluble proteins that only have a ligand binding moiety. While this may negate the immunosuppressive effects of the target ligand, the effects are transient and only localized to the transgenically modified cells. The benefit gained from certain embodiments include modification of T cells to not simply block but rather invert the effects of inhibitory molecules.

Certain embodiments further improve the concept of soluble protein decoys by combing a stimulatory moiety in addition to the ligand binding domain, as shown in FIG. 9B. Certain embodiments demonstrate that arming T cells with such “switch receptors” comprised of extracellular domains derived from endogenous inhibitory receptors (e.g. IL-4Ra, PD-1, Fas) and cytoplasmic signaling domains derived from stimulatory receptors (e.g. IL-7Ra, CD28, 41BB) enhances the survival, expansion and anti-tumor activity of T cells in vivo

Thus, embodiments herein encompass a novel Fas decoy molecule that is secreted by transgenic T cells at the tumor site upon antigen engagement, protecting not only transgenic cells but also bystander (non-modified) endogenous immune cells.

### Example 13: Modularity of Suppressive Immune Ligand Defense Systems (SILDS)

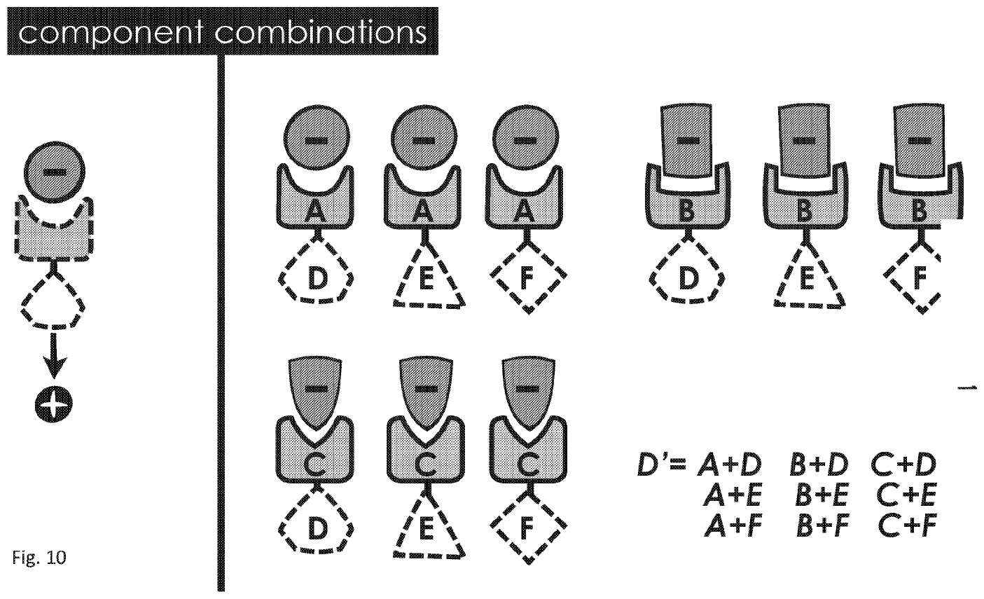

In some embodiments, the decoy described herein is a soluble fusion protein with two domains tethered by a Glycine-Serine linker. As shown in FIG. 10, the fusion proteins used herein may comprise different combinations of protein domain. The two domains can be categorized as a “negative” domain (which may be referred to as an “inhibitory protein domain”) and a “positive domain” (which may be referred to as an “activating protein domain”) wherein the negative domain functions to bind to the target immunosuppressive ligand and the positive domain functions to release immunostimulatory signals when activated. The negative and positive domains can be interchanged depending on the target ligand and preferred stimulatory signal. Such embodiments may be described herein as “Suppressive Immune Ligand Defense Systems” or “SILDS”

### Example 14: Theoretical Biological Activity of SILDS

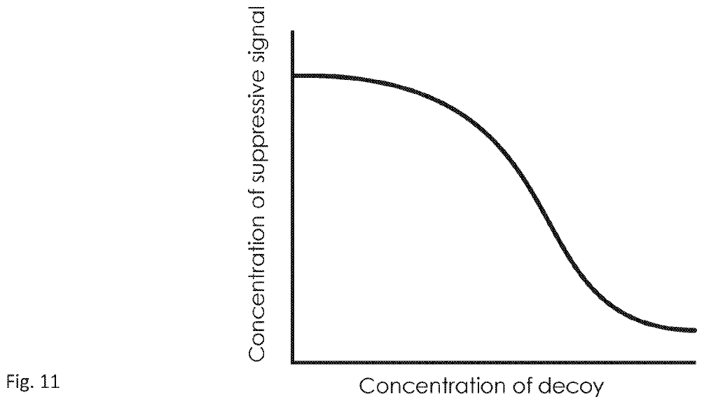

As shown in FIG. 11, inhibition of suppressive signal is concentration-dependent. Certain concentration of the decoy is required for inhibition of 50 percent of suppressive molecules. This concentration is known as inhibitory concentration 50 (IC50) and depends on the type of the suppressive molecule and the decoy.

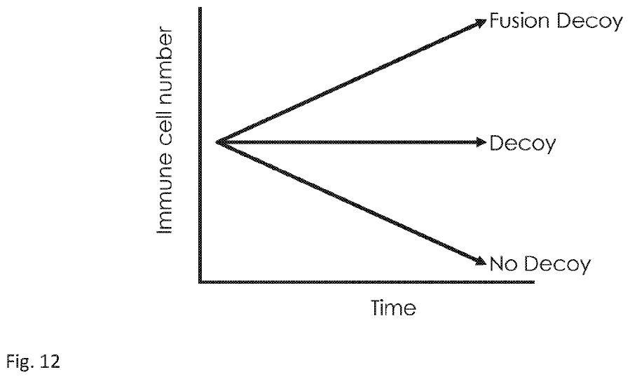

As shown in FIG. 12, in the presence of a suppressive signal and without any decoy present, the strength of immune response represented here by immune cell number declines. But, in the presence of the decoy, immune cells are protected from the destructive effects of suppressive signal and are able to persist. With fusion decoy present, immune cells are not only protected from the suppressive signal, but also activated receiving a stimulatory signal from the stimulatory moiety of the fusion decoy. Therefore, immune cells can grow beyond the number that they would grow in presence of non-fusion decoy.

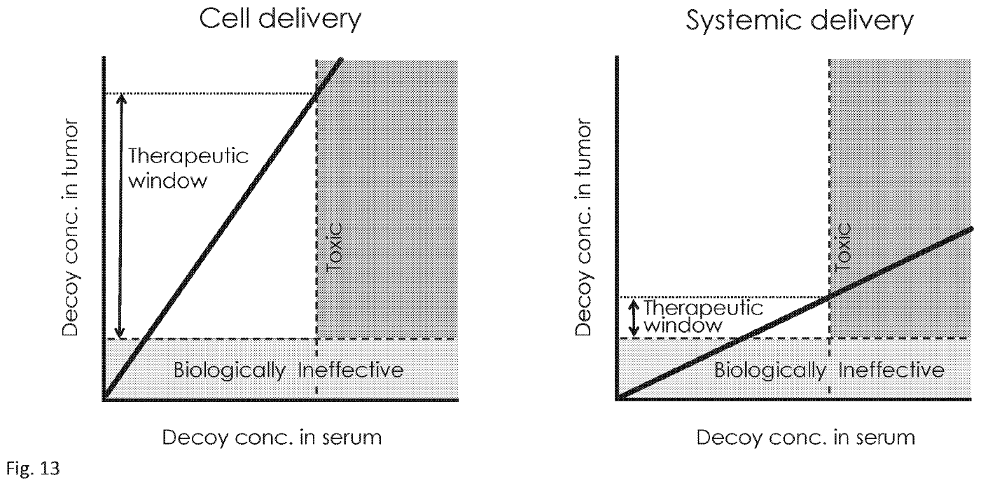

As shown in FIG. 13, there is an expected therapeutic window occurring in decoy concentrations above the threshold of biological ineffectiveness within the tumor, without exceeding toxic concentrations in serum by way of cell delivery (left) or systemic delivery (right). Excessive serum concentrations of exogenous proteins are potentially toxic beyond certain threshold (dark shadowed box). On the other hand, decoy concentrations in the tumor microenvironment below a certain threshold are biologically ineffective (light shadowed box). These two thresholds define the therapeutic window of the decoy.

In the case of systemic administration, the decoys concentration in tumor microenvironment is dependent on but lower than its concentration in serum (right). This makes the available therapeutic relatively narrow. On the other hand, cell delivery of the decoy provides higher concentrations in the tumor microenvironment while diffusion of a portion of the molecules into the serum results in a relatively lower systemic concentration. Hence, cell delivery of the decoy is expected to allow for a broader therapeutic window, which may be beneficial in certain embodiments.

### Example 15: Theorized Bystander Effect of SILDS

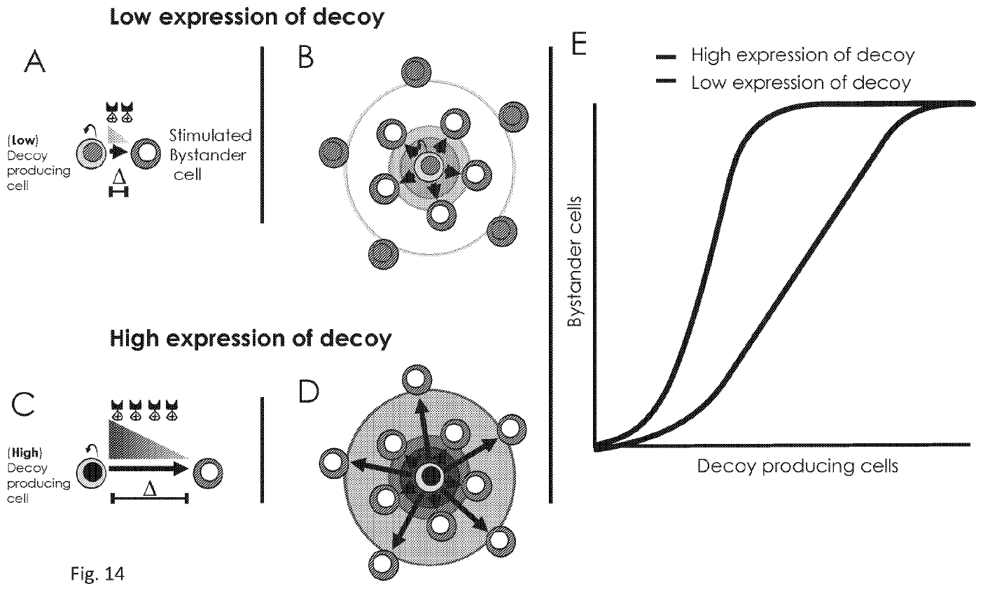

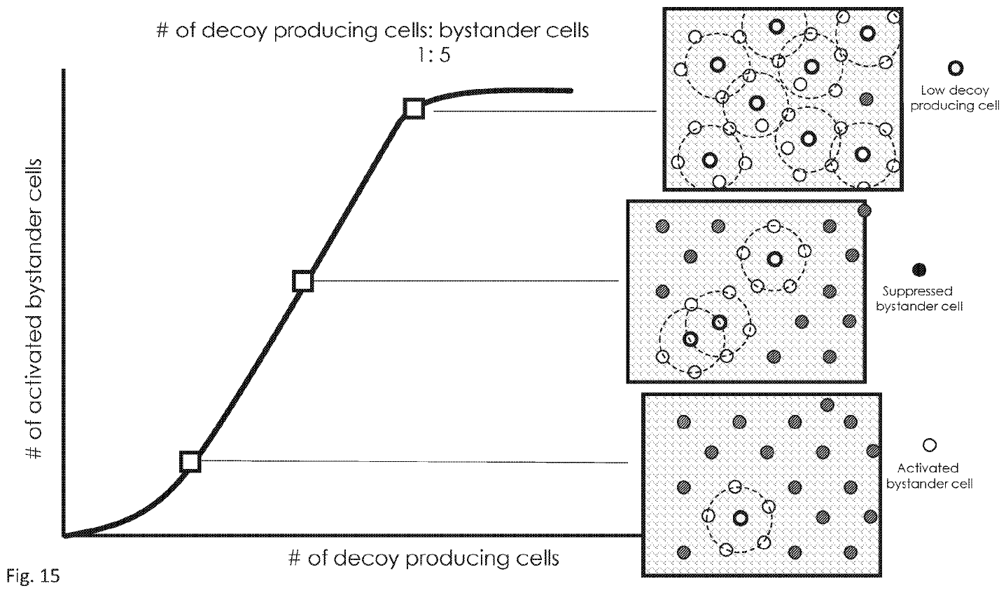

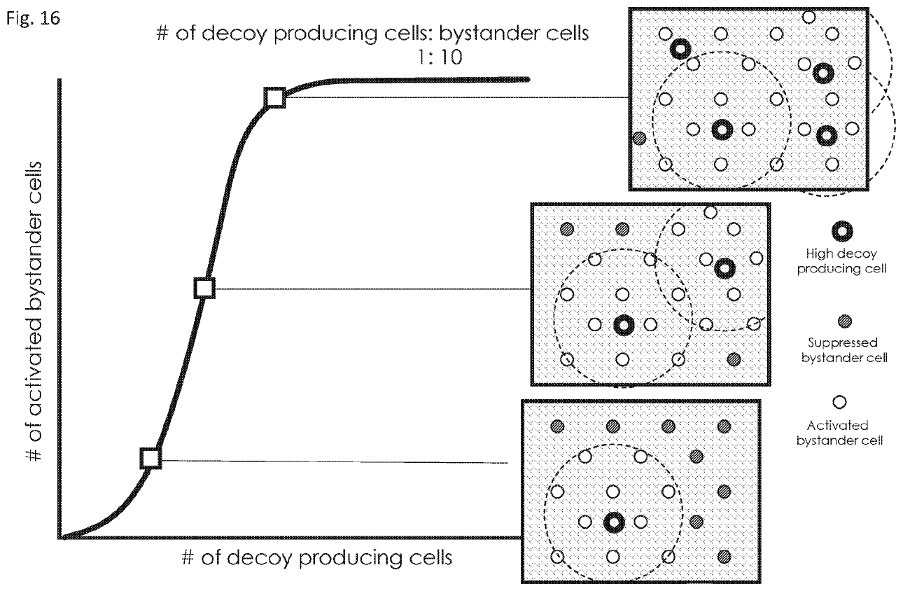

Cells expressing low levels of decoy reach a bystander cell within a short range (as shown in FIG. 14A) resulting in a stimulatory effect extending to nearby cells (as shown in FIG. 14B). Cells expressing high levels of decoy reach a bystander cell within a long range (as shown in FIG. 14C) resulting in a stimulatory effect extending beyond nearby cells (as shown in FIG. 14D). FIG. 14E shows the correlative comparison between the number of decoy producing cells and number of bystander cells. In certain embodiments, as shown in FIG. 15, the number of bystander cells activating for each present cell produces low amounts of decoy, including in a ratio of 1 to 5. In certain embodiments, as shown in FIG. 16, the number of bystander cells activating for each present cell produces high amounts of decoy, including in a ratio of 1 to 10.

### Example 16: Theorized Biochemical properties of SILDS

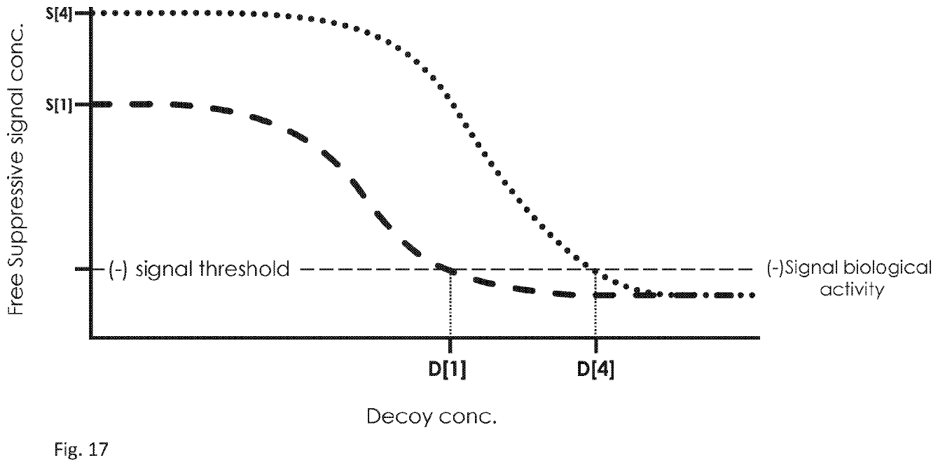

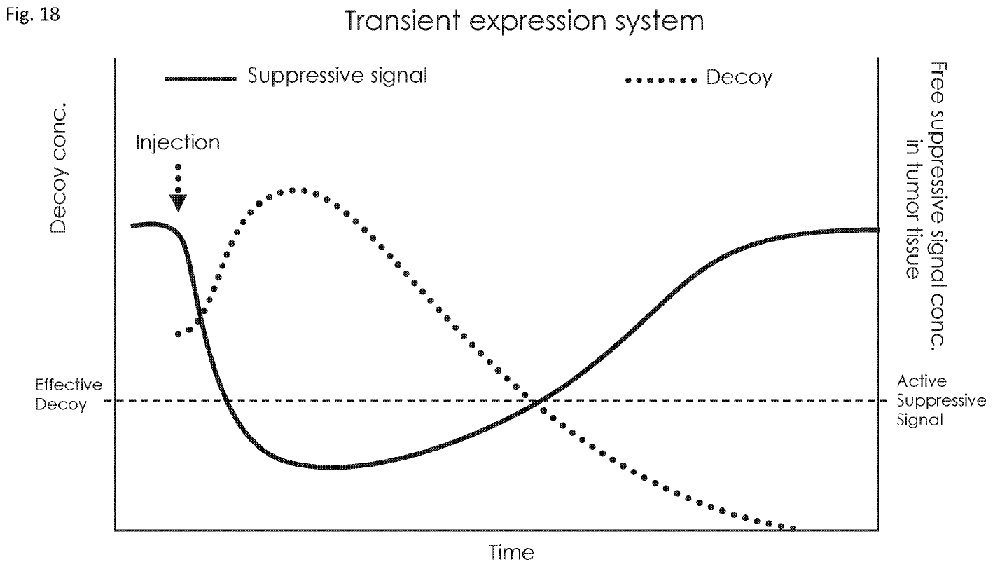

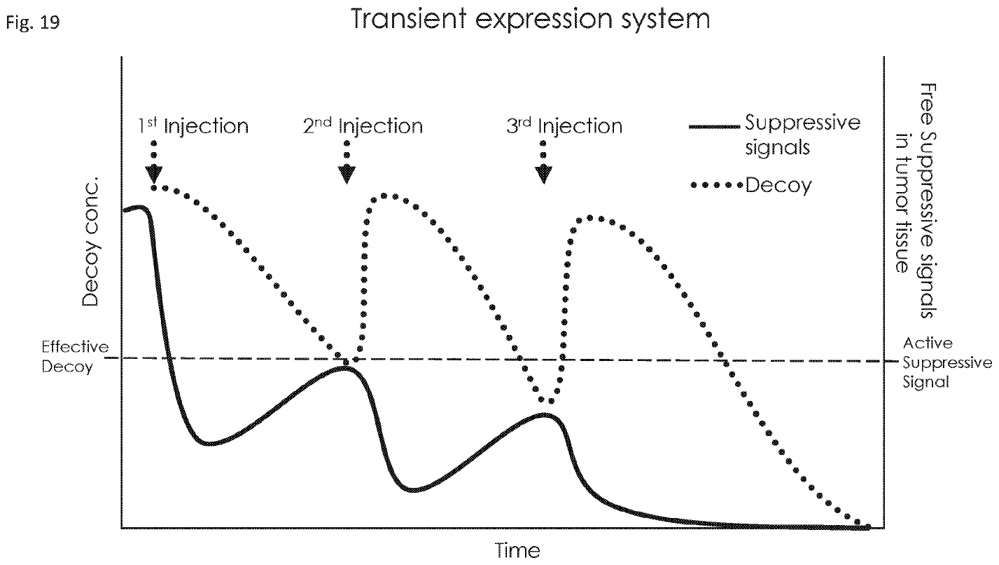

FIG. 17 demonstrates that concentrations of biologically active suppressive signals are controlled by a corresponding concentration of decoy. FIG. 18 demonstrates cells modified to transiently express decoy will temporarily control active suppressive signals in tumor tissue. FIG. 19 demonstrates that multiple administrations of cells modified to express decoy will permanently control active suppressive signals in tumor tissue.

### Example 17: Possible Derivations of SILDS

Cells isolated from donors in the following examples may be any type of cell, including peripheral blood mononuclear cells, placental and umbilical cord derived cells, lymphoid tissues derived cells, and tumor-infiltrating immune cells.

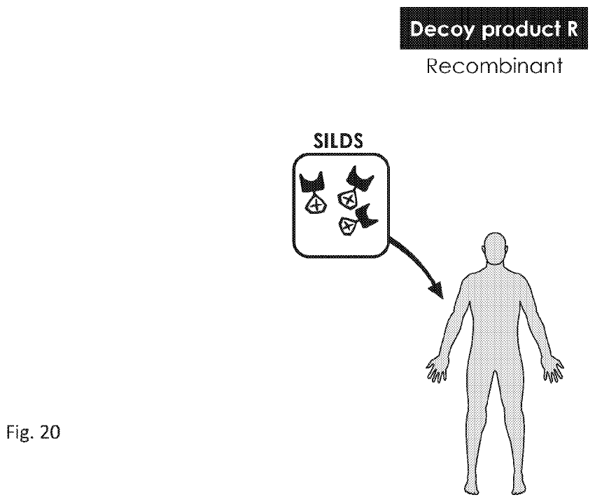

In certain embodiments, a decoy therapy, such as SILDS, includes decoy product manufactured from a recombinant source which can be derived from established engineered cell lines of mammalian origin or other organisms. In certain embodiments, the cells would then be modified to generate decoy that bind to the specific protein of interest, while providing a stimulatory signal to immune cells, and placed in media where they then release decoy throughout the duration of the culture. This media now contains a concentration of decoy which retains its engineered properties and comprises the product, which may be administered to the individual, as demonstrated in FIG. 20.

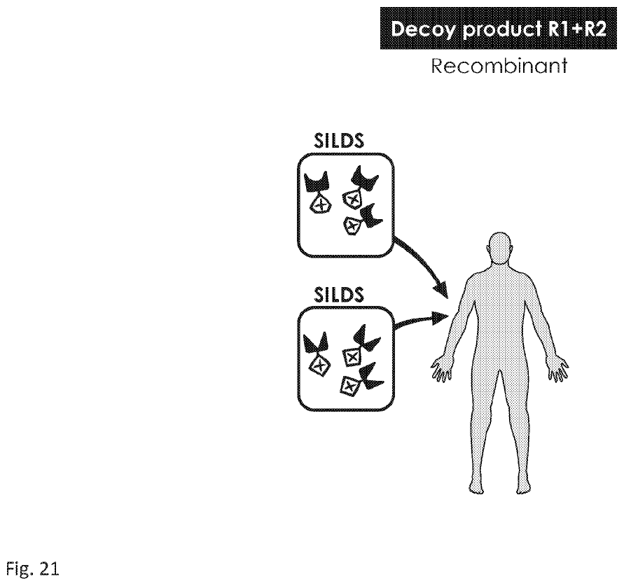

Certain embodiments use another form of decoy product manufactured from a recombinant source can be composed of a combination of more than one type of decoy derived from the same or different established engineered cell lines of mammalian origin or organisms, which are divided into groups. Each group is modified to generate a decoy that binds to a specific protein of interest while providing a stimulatory signal and placed in media where they release decoy throughout the duration of their culture. This media now contains a concentration of decoy which retains its engineered properties and comprises the product, which may be administered to the individual, as shown in FIG. 21.

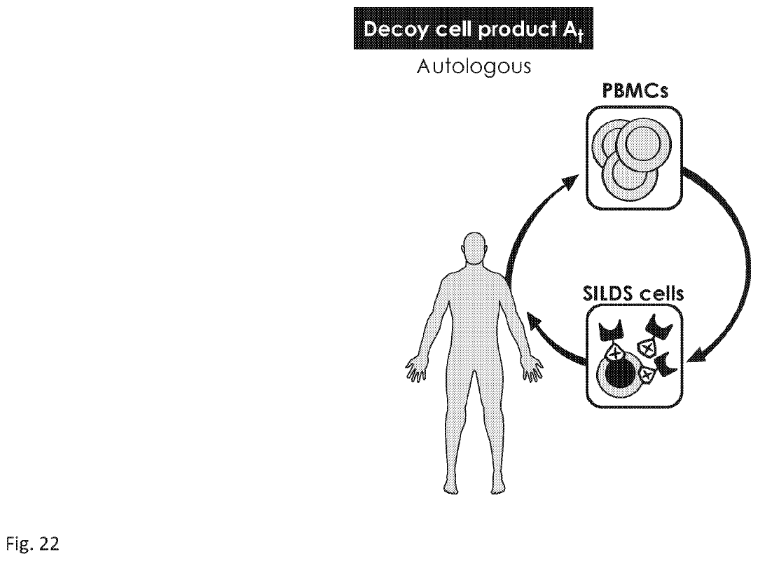

Certain embodiments encompass decoy product manufactured from an autologous source, which is derived from a patients cells that are engineered to produce decoy (Decoy cell product Aₜ). The patient to receive the product would have their PBMCs isolated, stimulated and modified to produce the decoy that bind to a specific protein of interest while providing a stimulatory signal. These decoy producing cells now comprise the product, which may be administered to the individual, as shown in FIG. 22.

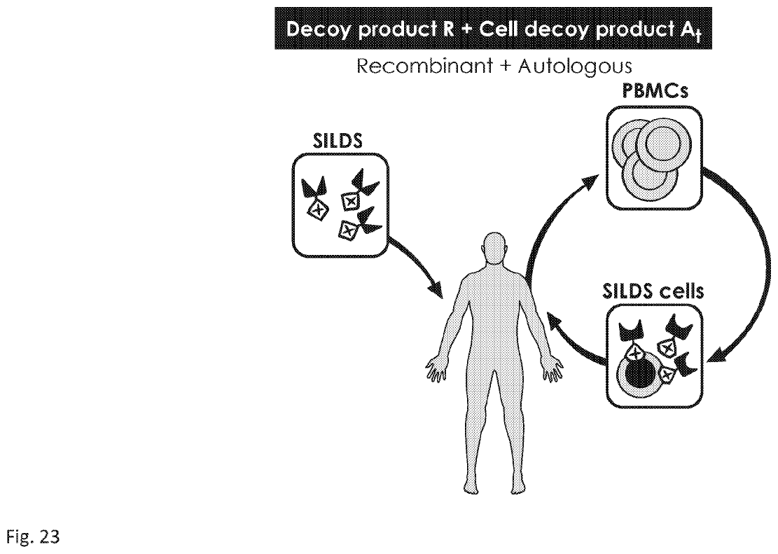

Certain embodiments encompass a decoy therapy that includes decoy product manufactured from a combination of more than one decoy derived from recombinant and autologous sources. The patient to receive the product would have their PBMCs isolated, stimulated and modified to produce decoy that binds to the specific protein of interest, while providing a stimulatory signal to immune cells. A different decoy is derived from established engineered cell lines of mammalian origin or organism that would then be modified to generate decoys decoy that binds to the specific protein of interest while providing a stimulatory signal to immune cells, and placed in media where they then release decoy throughout the duration of the culture. This media now contains a concentration of decoy that retains its engineered properties. Both the media of recombinant sourced decoy and autologously sourced decoy producing cells comprise the product, which may be administered to the individual, as shown in FIG. 23.

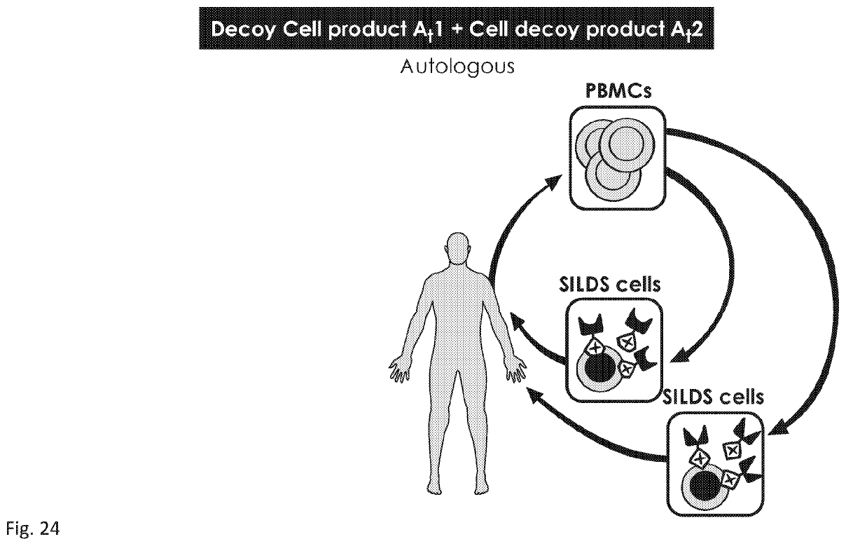

Certain embodiments utilize another strategy, wherein the decoy is generated from autologously derived cells, which are engineered to produce more than one decoy of interest. The patient to receive the product would have their PBMCs isolated, stimulated and divided into groups each of which is modified to produce a decoy targeting a different molecule while providing a different stimulatory signal. The decoy and/or the decoy producing cells may be administered to the individual as shown in FIG. 24.

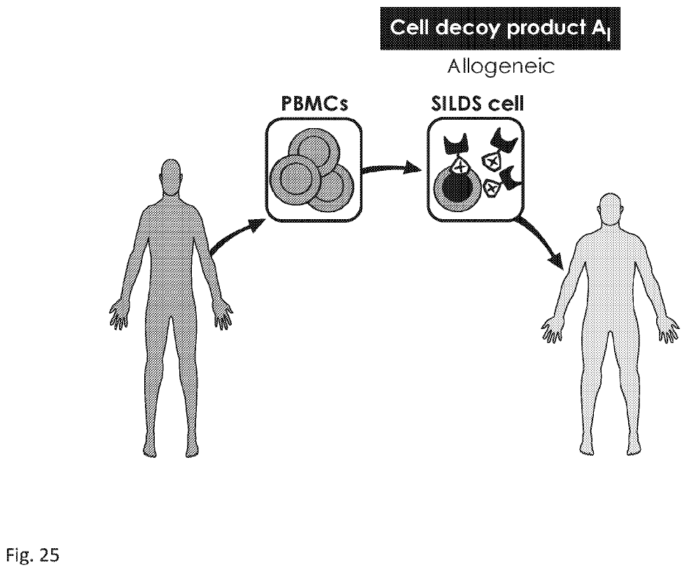

In certain embodiments, a decoy is generated using cells from a donor, which are engineered to produce the decoy product. First, the cells are isolated from a donor. The cells are stimulated and modified to produce the decoy, which is released as the product to the media. The product may share the same properties as an autologous engineered cells that produce the decoy therapy. As shown in FIG. 25. The cells can be administered to the patient, including as a conventional therapeutic infusion product. The cells may provide a benefit in the tumor microenvironment to enhance therapies.

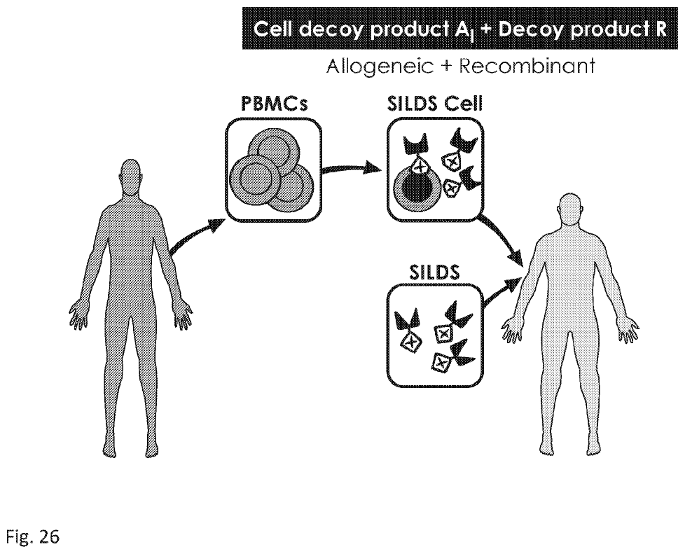

In certain embodiments, a decoy is generated using cells from a donor, which are engineered to produce the decoy product. First, the cells are isolated from a donor. The cells are stimulated and modified to produce the decoy, which is released as the product to the media. The product may share the same properties as an autologous engineered cells that produce the decoy therapy. In addition, a secondary decoy product targeting a different molecule and generate a different improvement as the decoy generate from autologous cells. This second decoy can be derived from established engineered cell lines (or modified organism that is used to generate biological products) that release the product to the media and produce a product that share the same properties as autologous engineered cells that produce the decoy therapy. As shown in FIG. 26, the decoy and cells can be administered to the patient, including as a conventional therapeutic infusion product. The cells and decoy may provide a benefit in the tumor microenvironment to enhance therapies.

Some embodiments encompass allogeneic source targeting. In certain embodiments, a decoy is generated using cells from a donor, which are engineered to produce the decoy product. First, the cells are isolated from the donor. The cells are stimulated and modified to produce the decoy, which is released as the product to the media. The product may share the same properties as an autologous engineered cells that produce the decoy therapy. Some embodiments employ recombinant technologies. As a non-limiting example, a secondary decoy product targeting a different molecule may be generated having a different improvement from the decoy generated from autologous cells. This second decoy can be derived from established engineered cell lines (or modified organism that is used to generate biological products) that release the product to the media and produce a product that share the same properties as autologous engineered cells that produce the decoy therapy.

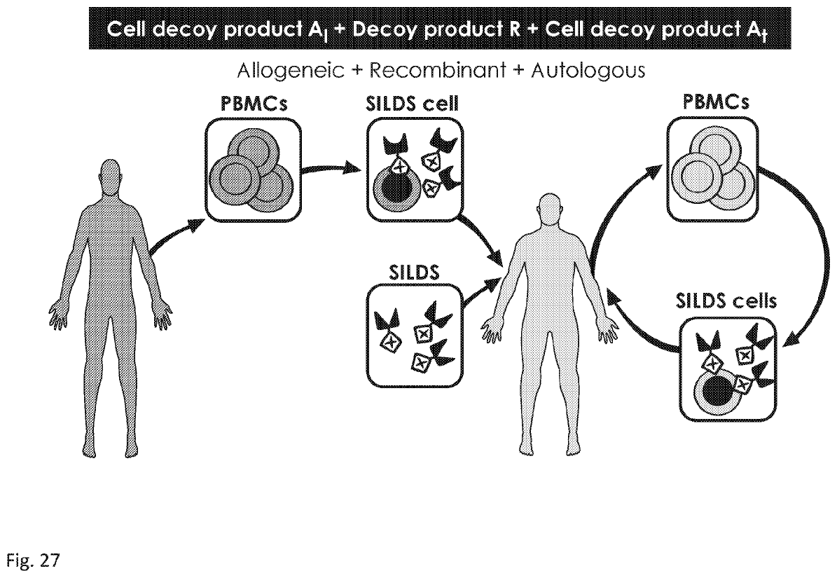

Certain embodiments encompass combinations of allogenic and autologous cells, either or both of which may be engineered to produce the product. For example, first the cells are isolated from a donor, then stimulated and modified to produce the decoy. As shown in FIG. 27, the cells and the decoy are administered to the same donor or an individual that is different from the donor. The cells, decoy, and/or product can be administered to the individual as a conventional therapeutic infusion product. The cells may provide a benefit in the tumor microenvironment to enhance therapies.

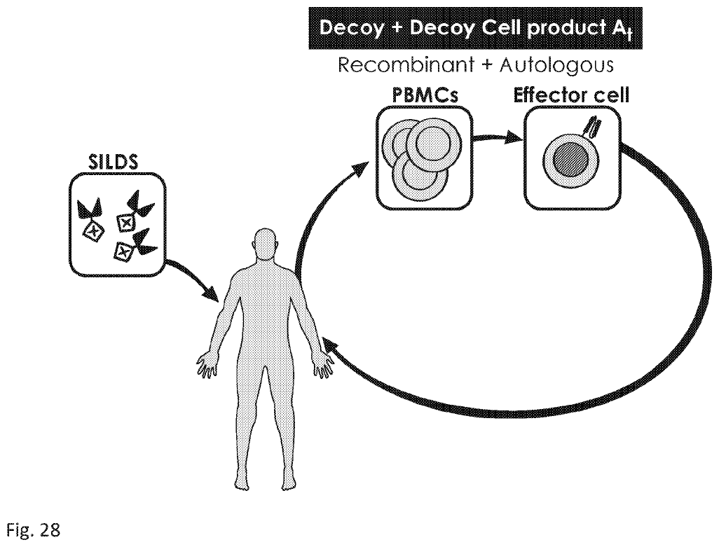

In some embodiments, the decoy product is manufactured from a recombinant source. The product can be derived from established engineered cell lines (or modified organism that is used to generate biological products) that release the product to the media and the product may share the same properties as an autologous engineered cells that produce the decoy therapy. As shown in FIG. 28, the cells and the decoy can be administered to the individual, including as a conventional therapeutic infusion product.

Certain embodiments use engineered autologous cells to achieve the desire effector cells. These cells are isolated from a donor, then stimulated and modified to produce the desired effector cells. These cells may be administered to the same donor from which the cells were isolated. The cells may receive the benefit from the decoy product in the tumor microenvironment to improve the efficacy of the therapies.

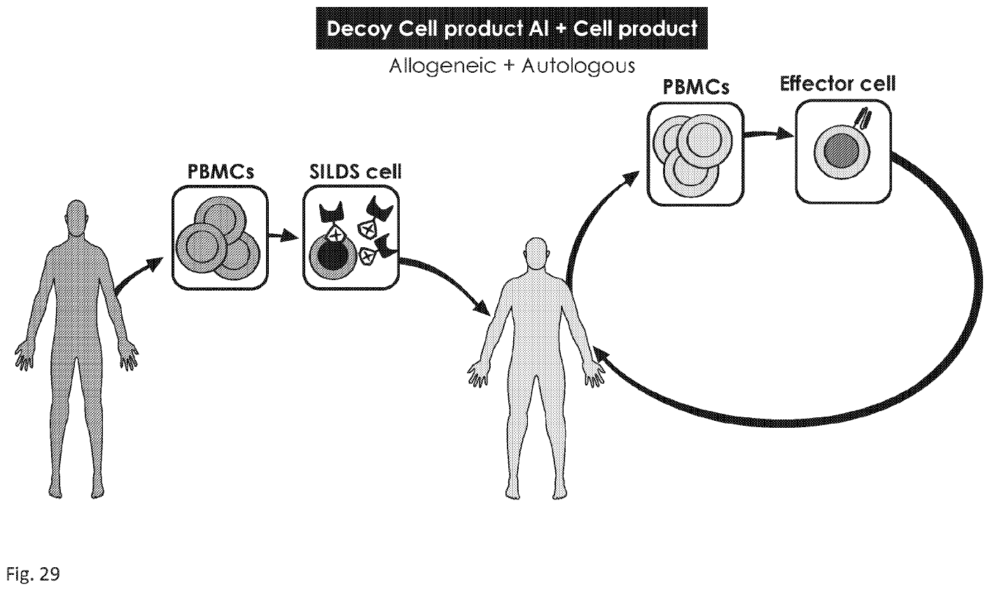

Certain embodiments generate the decoy therapy using cells from a donor. The cells are engineered to produce the decoy product. First the cells are isolated from the donor, they are stimulate and modified to produce the decoy, that release the product to the media and the product that share the same properties as an autologous engineered cells that produce the decoy therapy. As shown in FIG. 29, the effector cells and the cells producing the decoy can be administered to an individual, including as a conventional therapeutic infusion product.

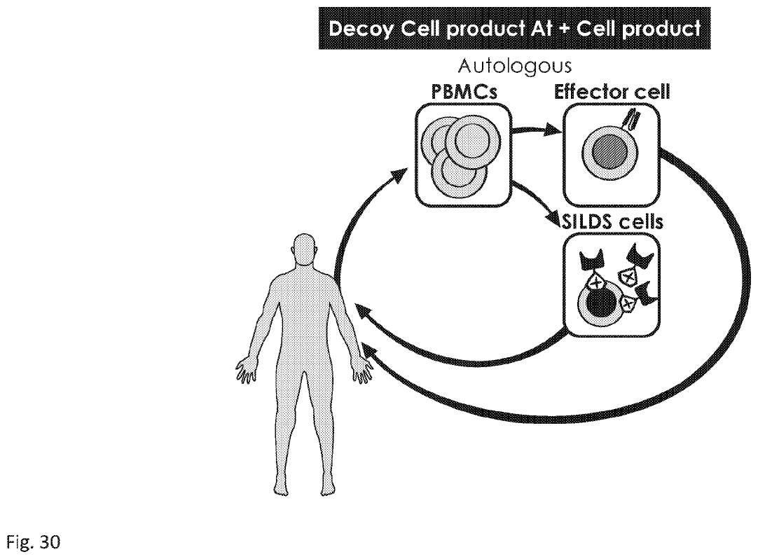

In some embodiments, a decoy therapy is generated using autologous cells that are engineered to generate the decoy product along with effector cells that receive benefit of the decoy. First, cells are isolated from a donor. The cells are then are stimulated, and one group of cells is modified to produce the decoy and another group of cells is used to generate the effector cells. As shown in FIG. 30, the effector cells and the cells producing the decoy are administered to the same donor from which the cells were isolated. The decoy cells may improve the effector cells function, including to enhance therapies.

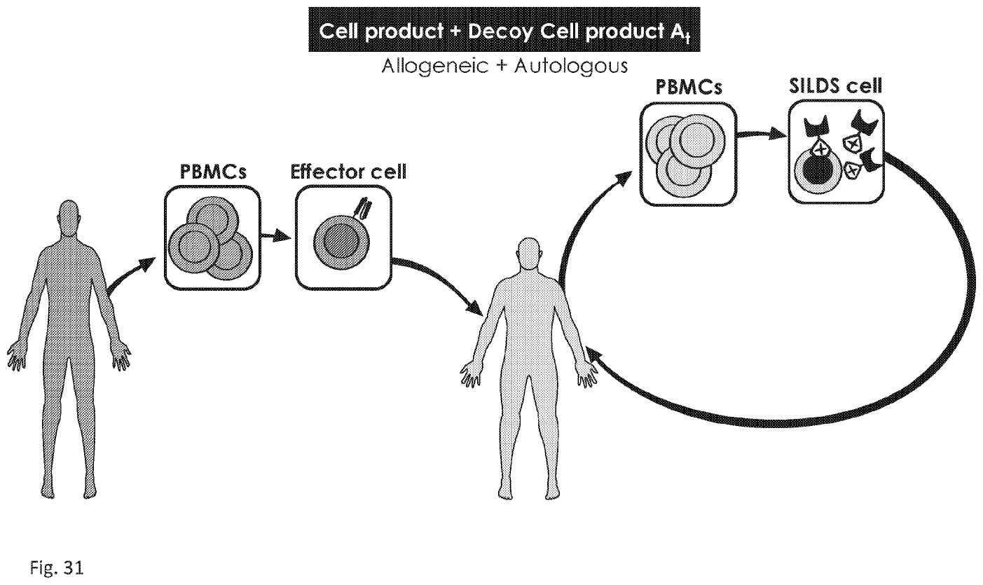

Some embodiment generate a decoy therapy is using autologous cells, which are engineered to produce a decoy product. First, cells are isolated from a first donor. The cells are then stimulated and modified to produce a decoy. The cells are administered to the same donor from which they were isolated. Further, cells from a different donor are engineered to become the desire effector cells. These cells are isolated from a selected donor, stimulated, and modified to produce the desired effector cells. As shown in FIG. 31, the effector cells and the cells producing the decoy are administered to the first donor. The effector cells may receive a benefit from the decoy producer cells in the tumor microenvironment to improve the efficacy of the therapy.

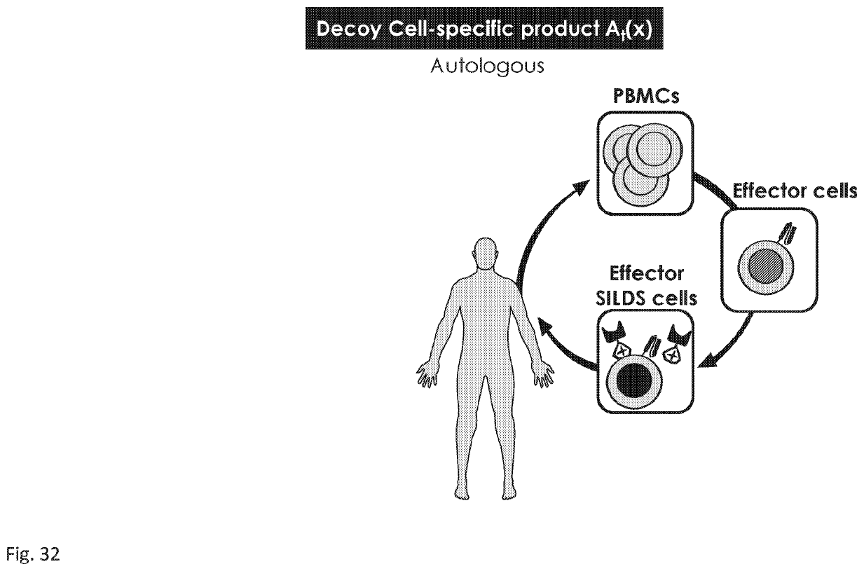

Some embodiments generate a decoy therapy using autologous cells, which are engineered to be desired effector cells and the decoy producer cells. First, the cells are isolated from the donor. The cells are then stimulated and modified to be the effector cells. The cells are then engineered to produce a decoy. Such cells can target different molecules and improve the effector cells function. The cells are also engineered to be effector cells. As shown in FIG. 32, the cells are administered to the same donor where from which they were isolated. The cells may provide a benefit in the tumor microenvironment to enhance therapies.

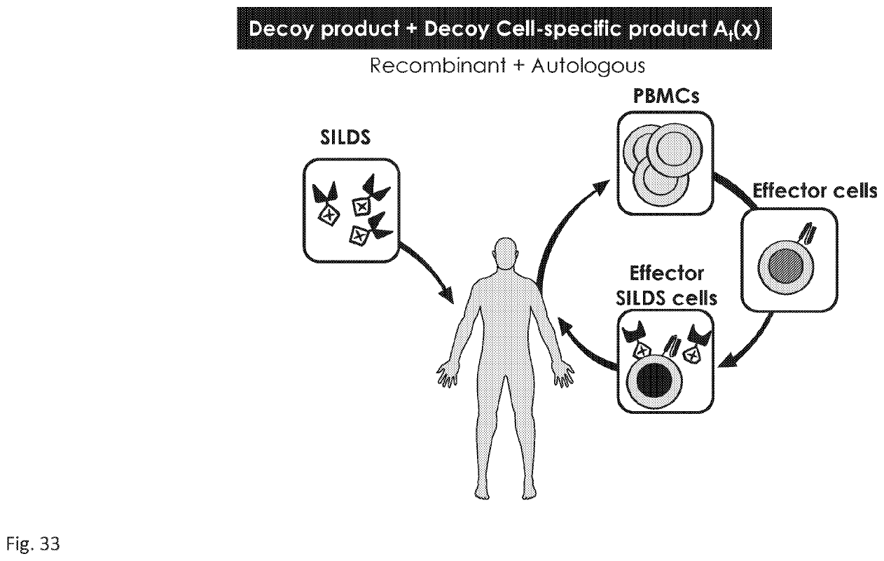

In certain embodiments, decoy product are also manufactured from a recombinant source. The product can be derived from established engineered cell lines (or modified organism that is used to generate biological products) that release the product to the media and produce a product that share the same properties as autologous engineered cells that produce the decoy therapy. As shown in FIG. 33, the decoy product and the engineered effector, decoy producing cells can be administered to an individual, including as a conventional therapeutic infusion product. The cells may provide a benefit in the tumor microenvironment to enhance therapies.

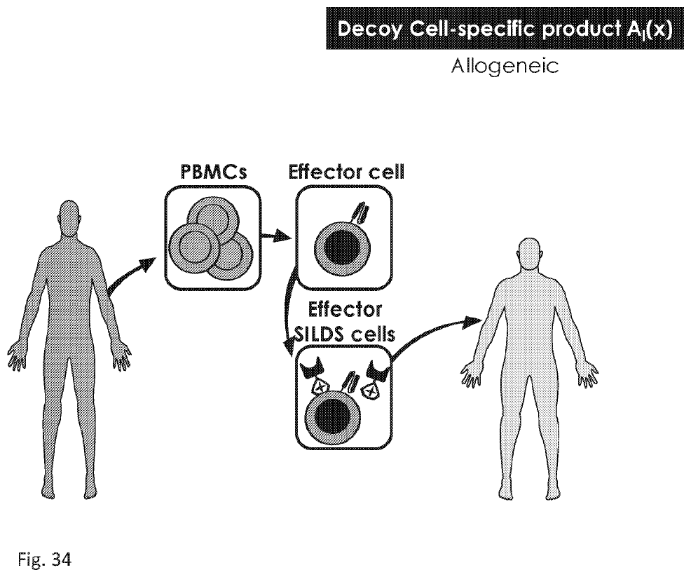

Certain embodiments generate a decoy therapy using allogeneic cells from a compatible donor. The cells are engineered to become a desired effector cell and produce the decoy. First, the cells are isolated from a donor. Then the cells are stimulated and modified to be the desired effector cell. The cells are then engineered to produce the decoy. The final engineered cells share the similar properties as an autologous engineered cells that produce the decoy therapy. As shown in FIG. 34, the cells can be administered to an individual, including as a conventional therapeutic infusion product. The cells may provide a benefit in the tumor microenvironment to enhance therapies.

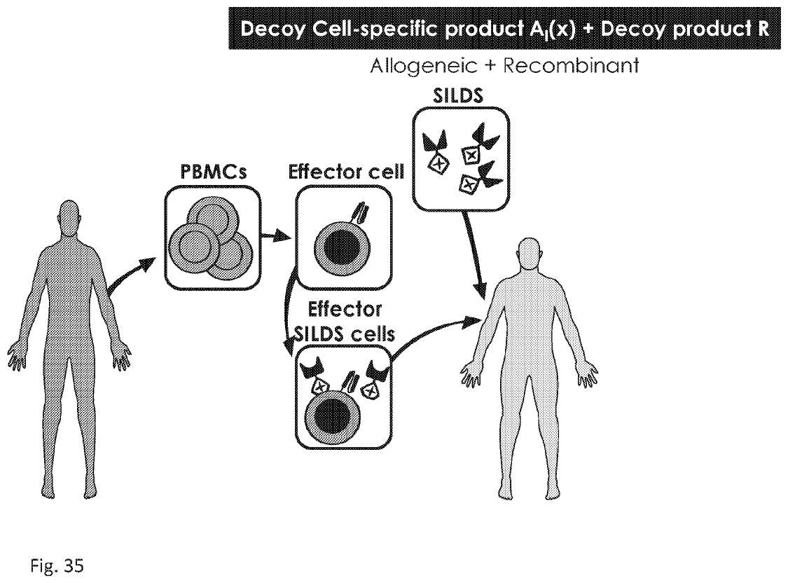

In certain embodiments, a decoy product can also be manufacture from recombinant sources. The product can be derived from established engineered cell lines (or modified organism that is used to generate biological products) that release the product to the media and produce a product that share the same properties as autologous engineered cells that produce the decoy therapy. As shown in FIG. 35, the cells and decoy product can be administered to an individual, including as a conventional therapeutic infusion product. The cells may provide a benefit in the tumor microenvironment to enhance therapies.

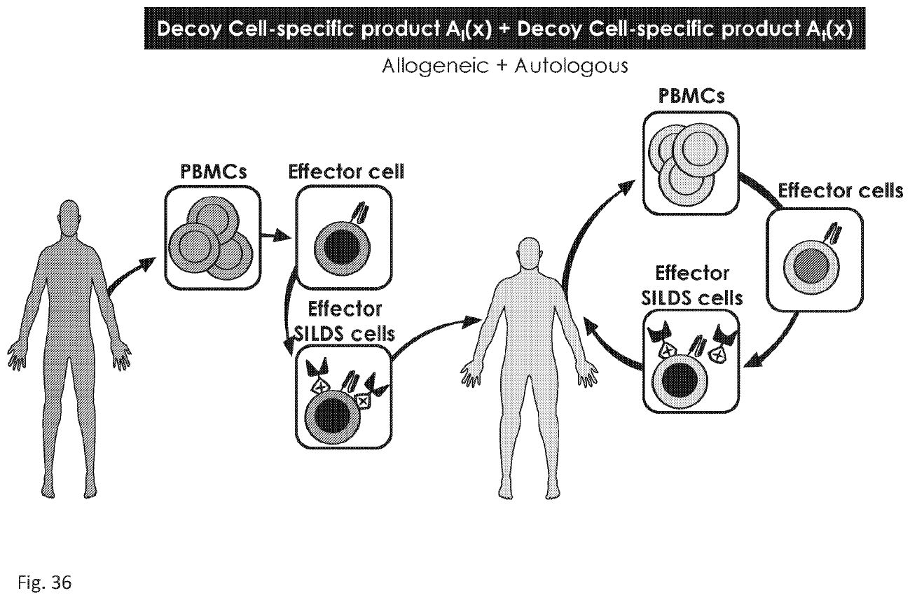

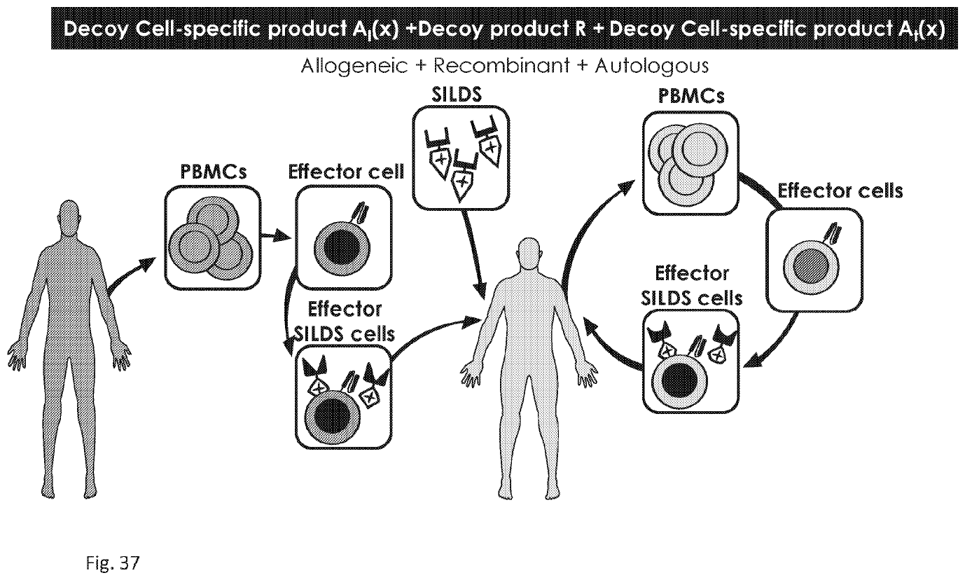

Certain embodiments generate a decoy therapy using allogeneic cells from a compatible donor. The cells are engineered to be desired effector cells and also produce the decoy. First, the cells are isolated from the donor. The cells are then stimulated and modified to be the desire effector cell. The cells are then engineered to produce the decoy. The final cell product may share similar properties as an autologous engineered cells that produce the decoy therapy. Also generated is a decoy therapy using autologous cells engineered to be both effector cells and the decoy producer cells. First, the cells are isolated from the donor. The cells are then stimulated and modified to be the desire effector cell. The cells are then engineered to produce the decoy. The cells can target different molecules and improve the effector cells function. As shown in FIG. 36, both the allogeneic and autologous effector, decoy producing cells are administered to an individual, including as a conventional therapeutic infusion product. The cells may provide a benefit in the tumor microenvironment to enhance therapies. In certain embodiments, as shown in FIG. 37, decoy products manufactured from recombinant sources (including as described herein), are also administered with the allogeneic and autologous effector, decoy producing cells to an individual. The cells and decoy products may provide a benefit in the tumor microenvironment to enhance therapies.

### Example 18: Examples of Cells Producing Decoys

In embodiments encompassing a decoy therapy, effector cells can by in combination with any decoy product described herein. The decoy may improve the effector cell function, including in a tumor microenvironment as well as improving other effector cell products function that can be affected by the same tumor microenvironment.

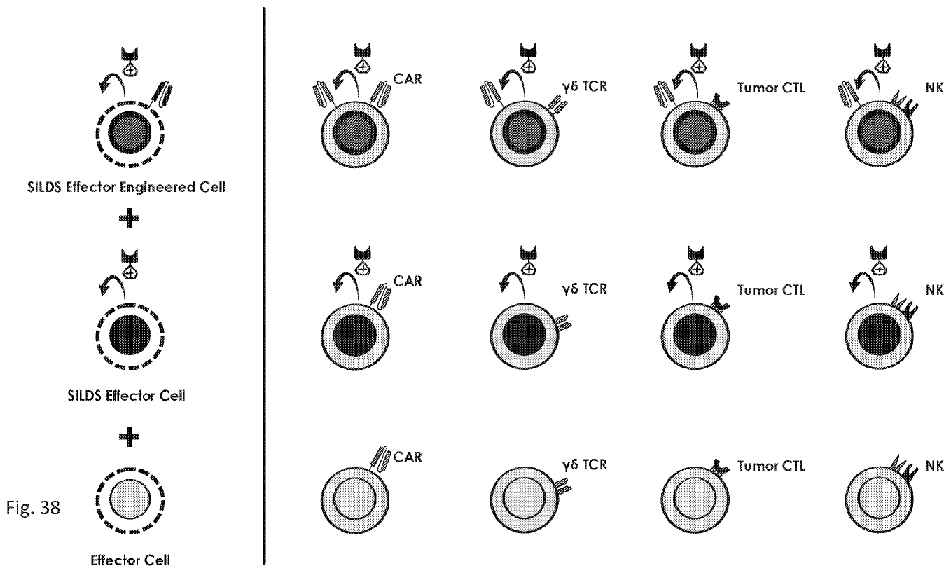

Any cell to be engineered to generate the desire effector cell may either produce decoy or not produce decoy, including any immune cells derived from an individual to be administered therapies described herein, from a single donor, rom more than one donor, or a combination thereof. As shown in FIG. 38, effector cells and/or decoy producing cells encompassed herein may be generated from cells including, but is not limited to, CAR T cells, T6 TCRs, tumor CTLs, NK cells, or other cellular platforms used for adoptive cell immunotherapy.

### Example 19: Manufacture of transient expressing decoy T cells

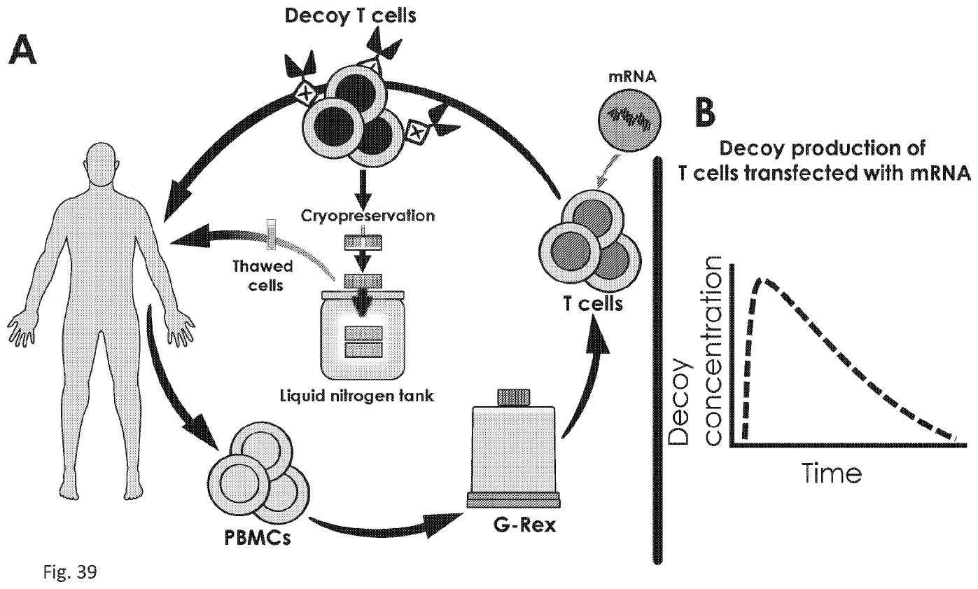

Certain embodiments concern the manufacture cells, such as T cells, producing a decoy. In such embodiments, first PBMCs from an individual are collected and isolated. The PBMCs are then stimulated to get the desired population and expand to get a large number of the cells. The cells are placed in optimal conditions, which are known to one skilled in the art and may include a G-Rex® or other suitable platform, to allow the cells to be engineered with a genetic construct to produce the decoy product. The genetically modified cells producing decoy may then be administered to the individual and/or cryopreserved for future administration to any individual. An embodiment of this method is shown as FIG. 39A. As shown in FIG. 39B, the production of the decoy by the T cells transfected with mRNA may decrease over the time; at the beginning the expression is higher, then as the amount of mRNA decreases inside the cell, the expression of the decoy molecule also decreases.

### Example 20: Theorized Biological Behavior of SILDS

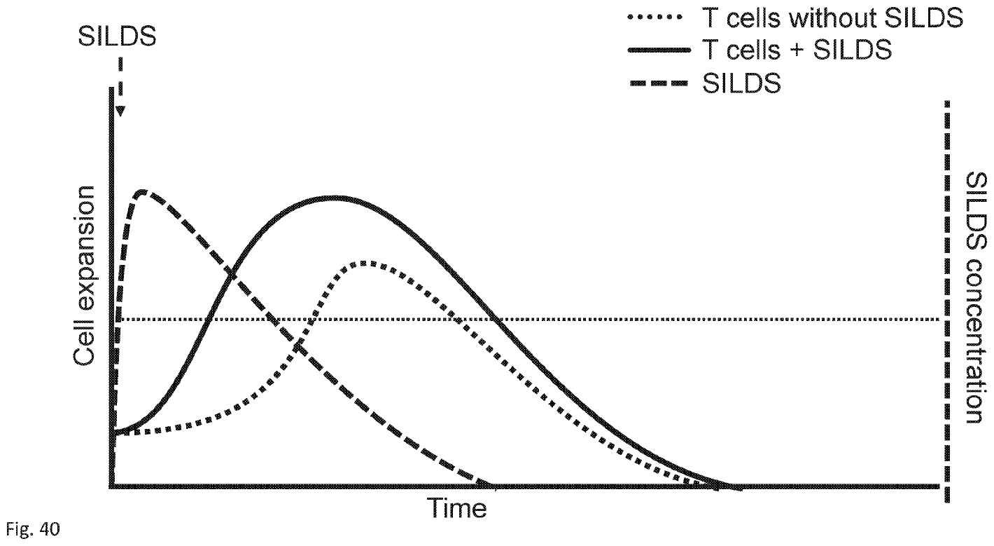

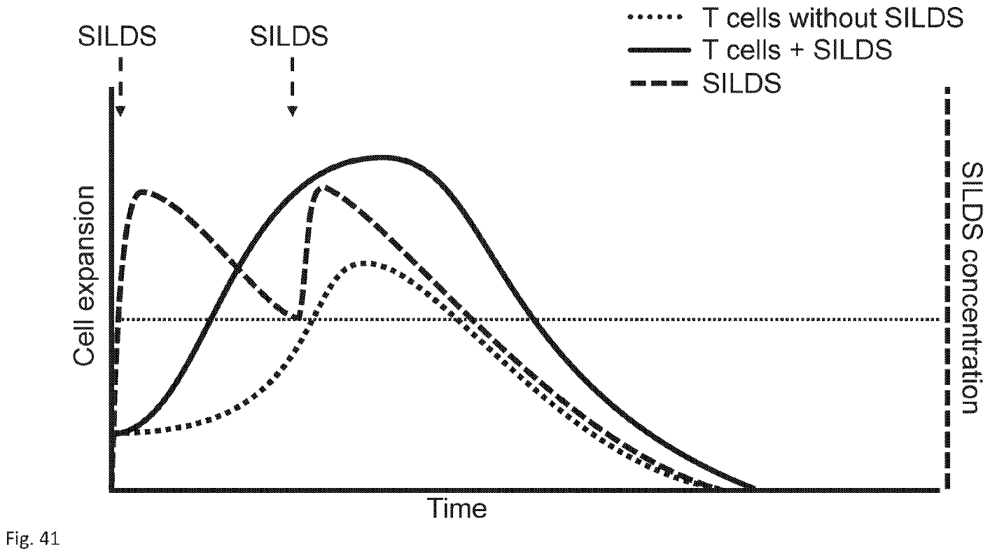

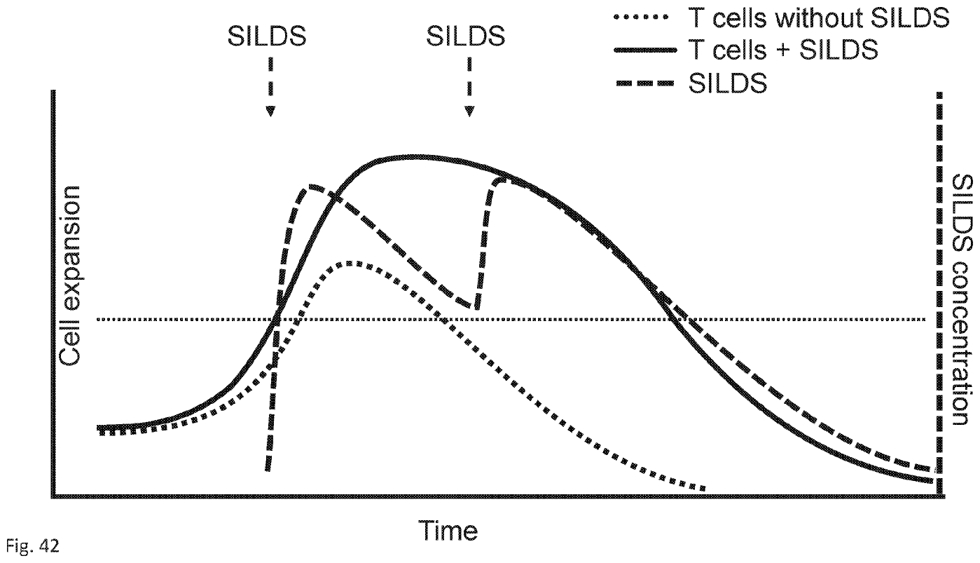

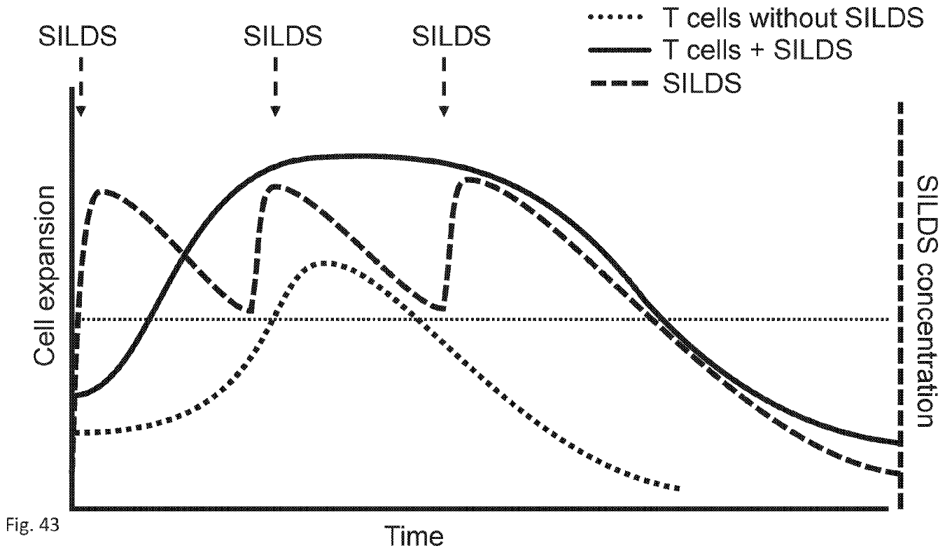

T cells expand more quickly and more robustly when decoy is introduced at the beginning of culture than culture without decoy (FIG. 40). T cells continue to expand when decoy is introduced at the beginning and during culture and/or when introduced multiple times during culture (FIGS. 41-43).

### Example 21: Possible Therapeutic Dosing of SILDS

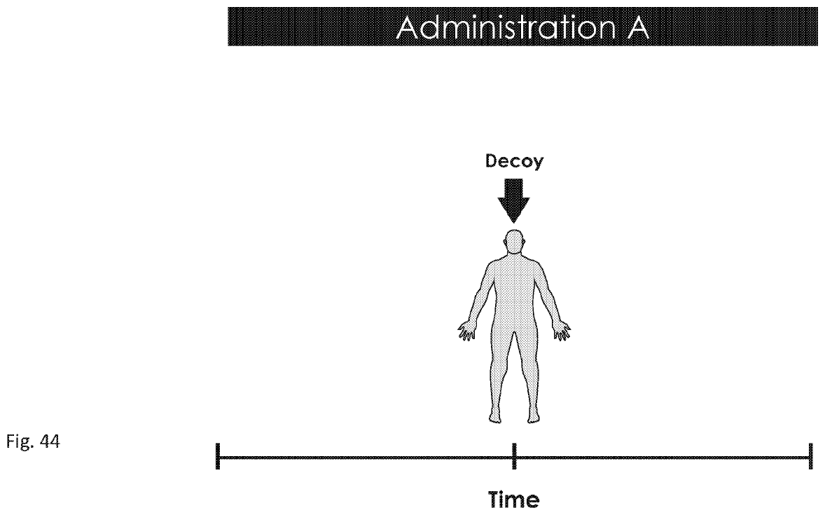

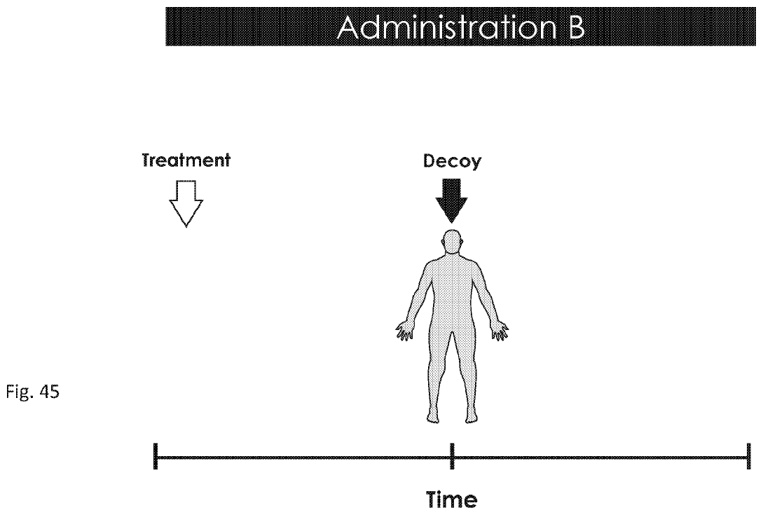

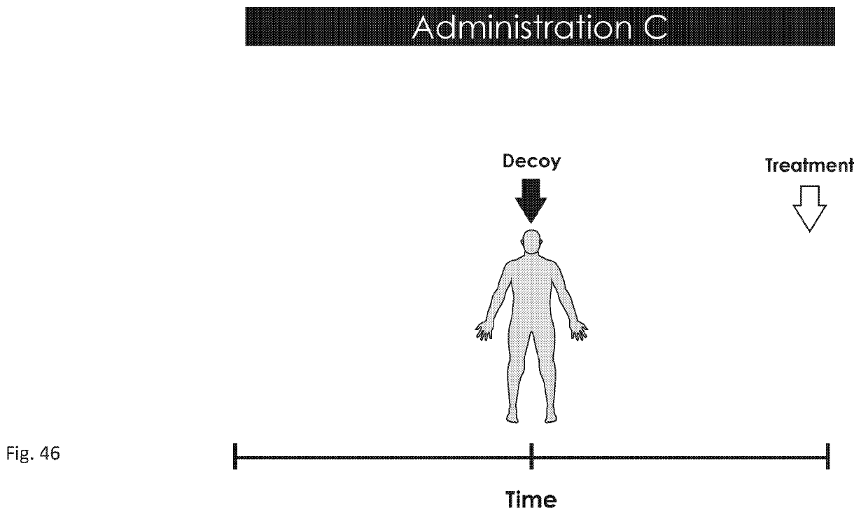

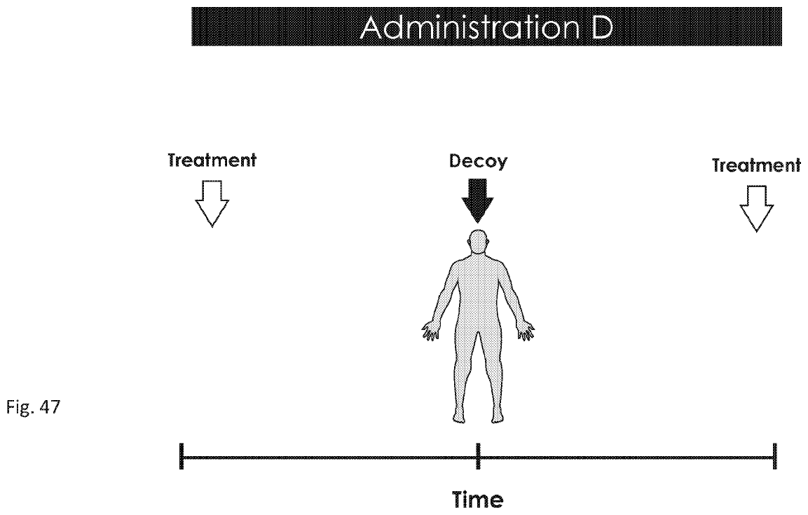

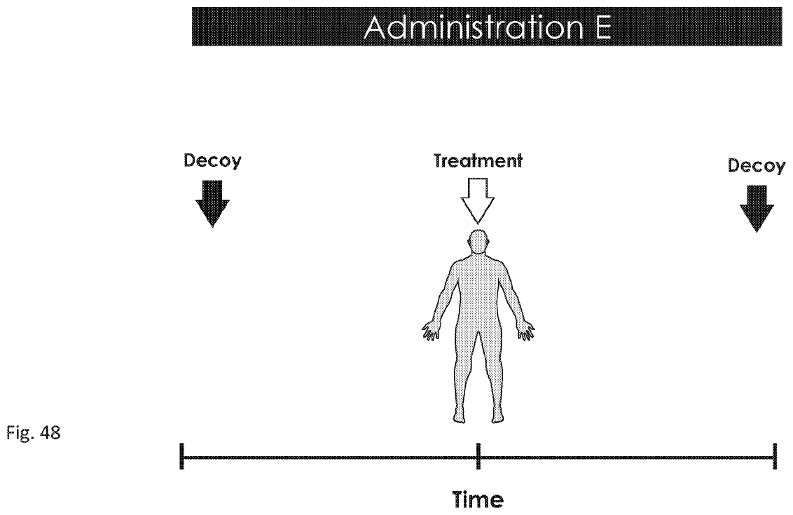

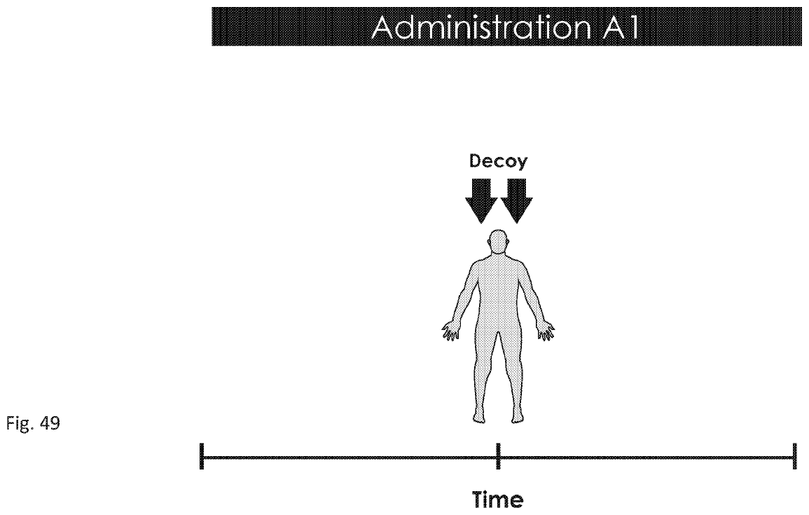

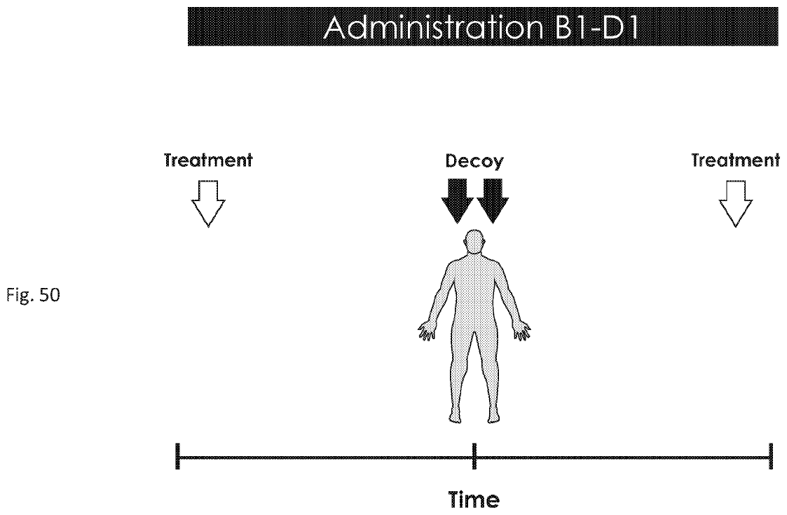

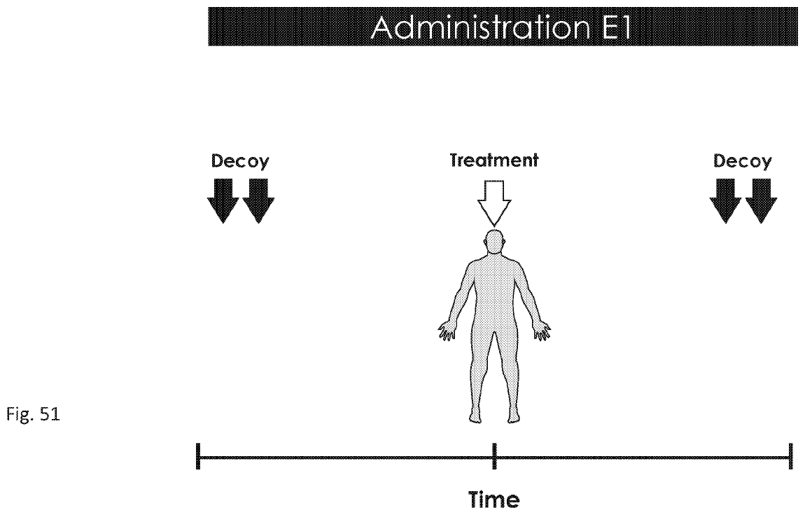

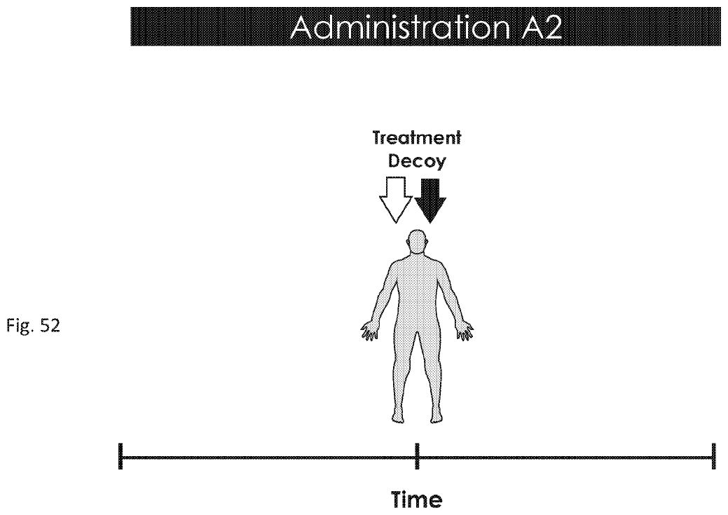

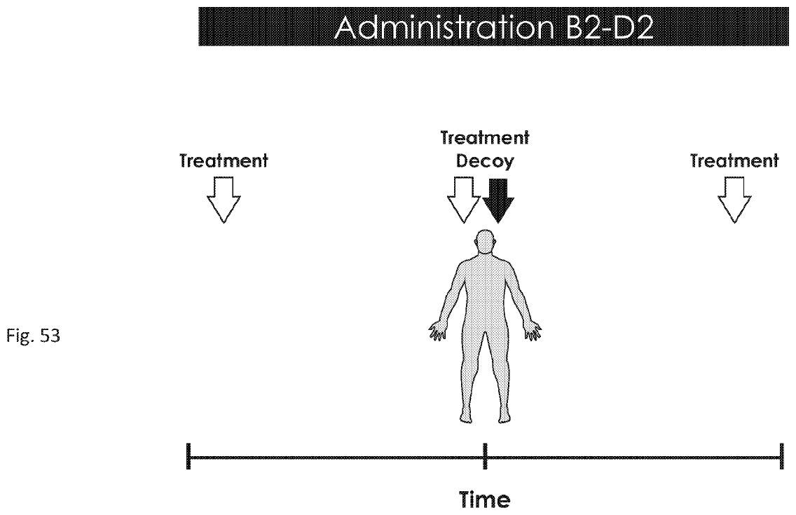

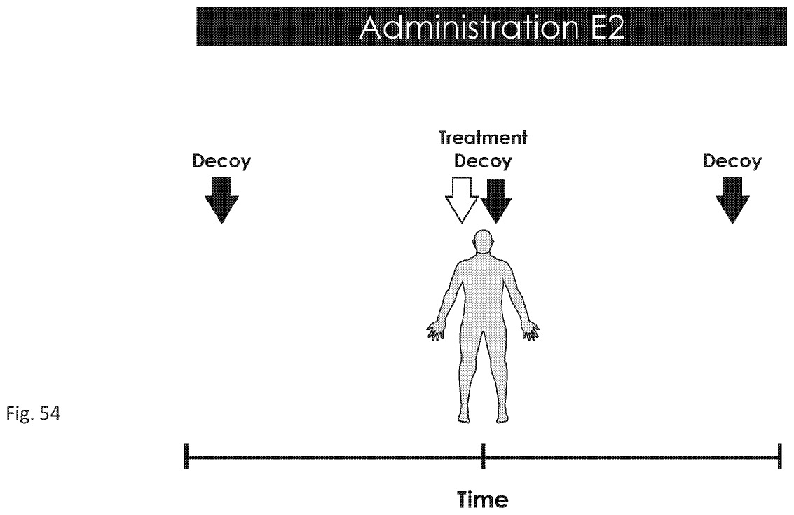

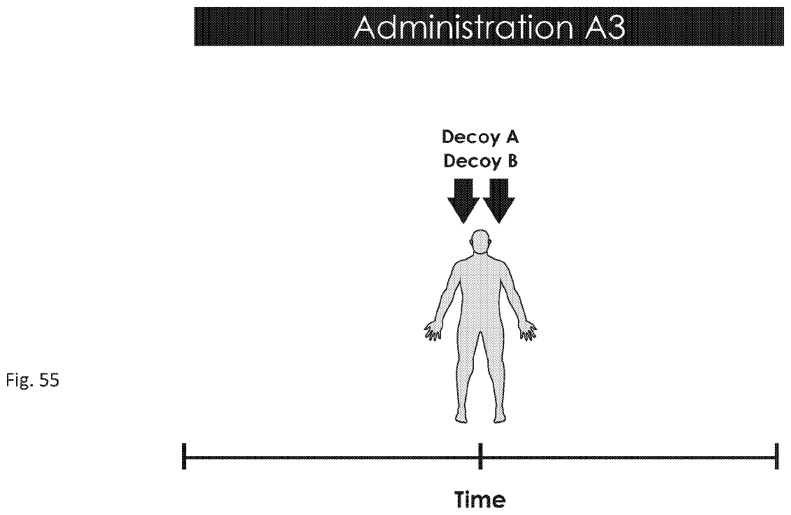

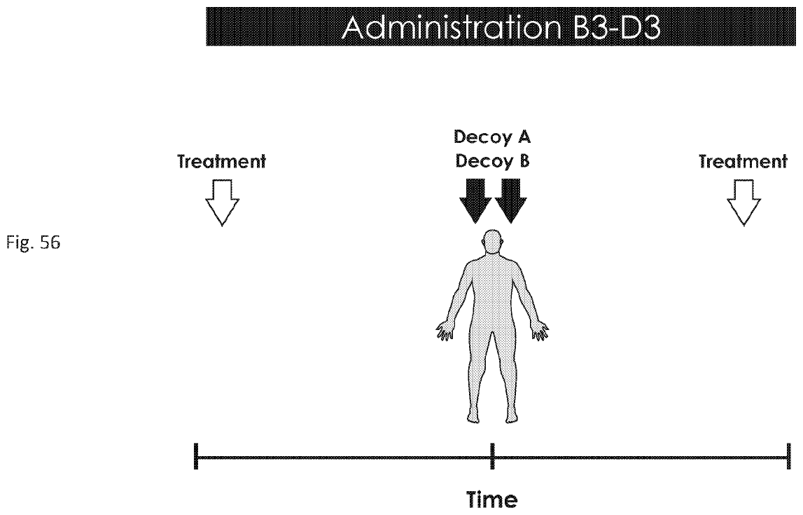

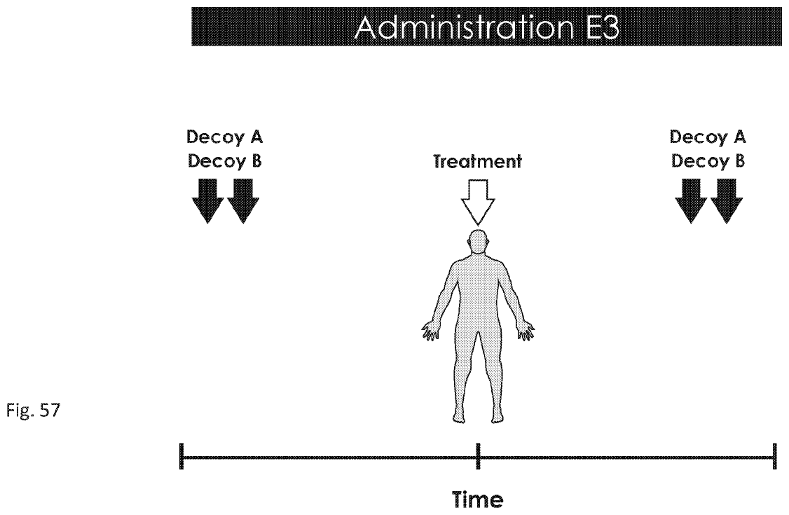

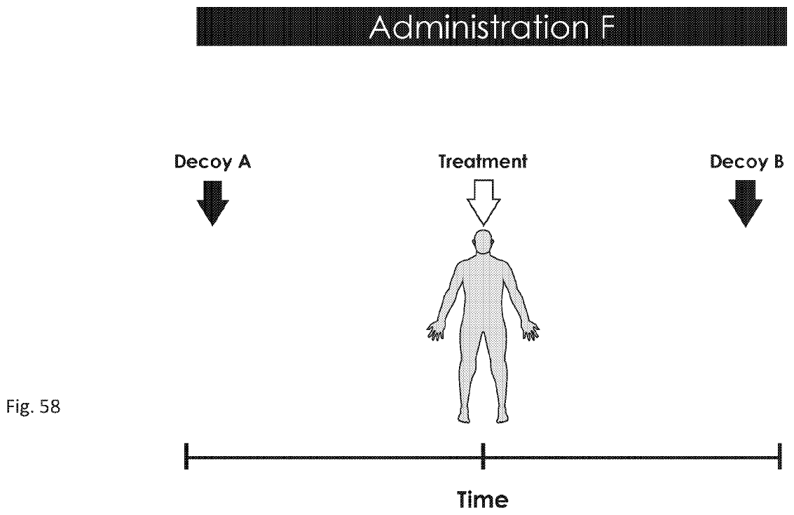

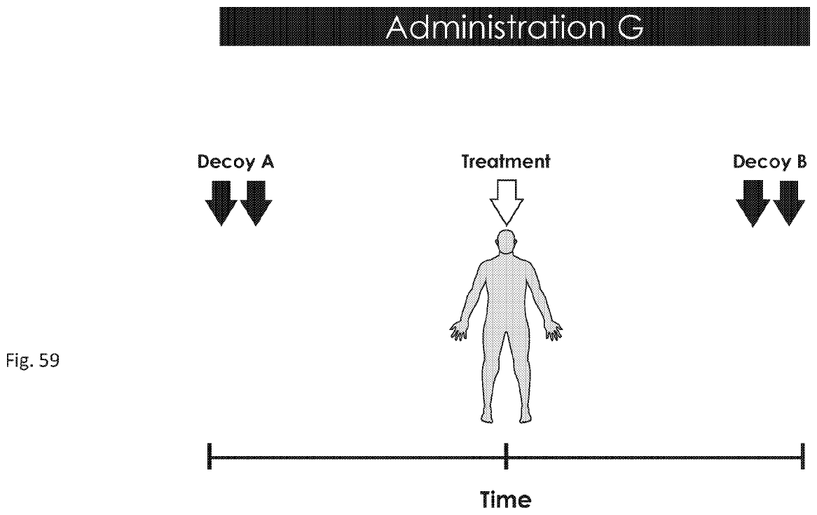

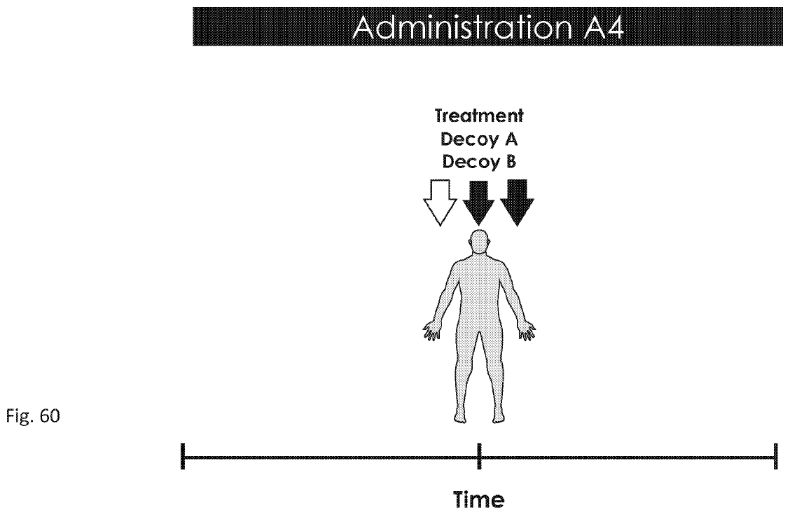

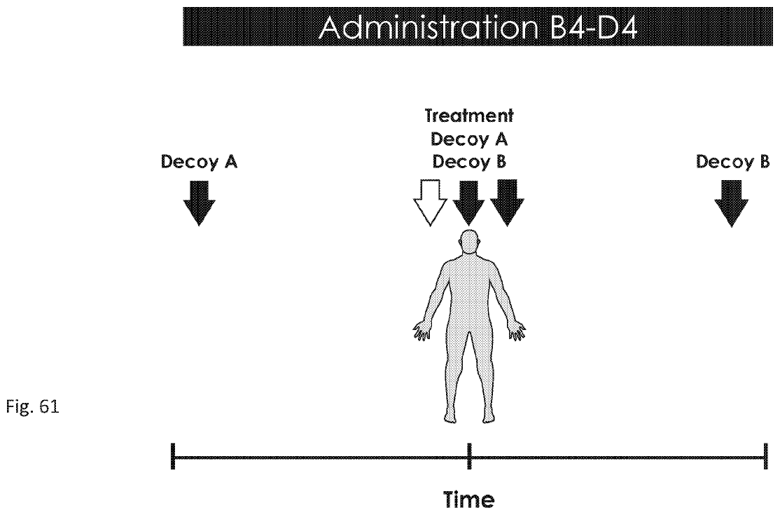

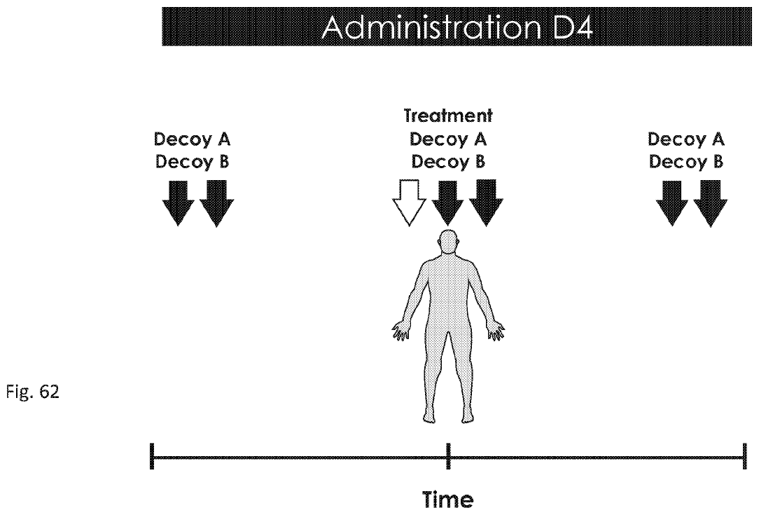

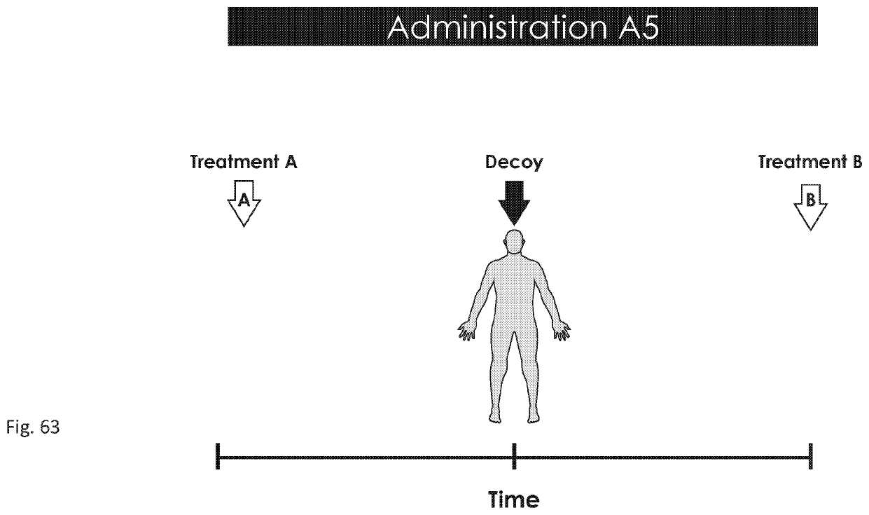

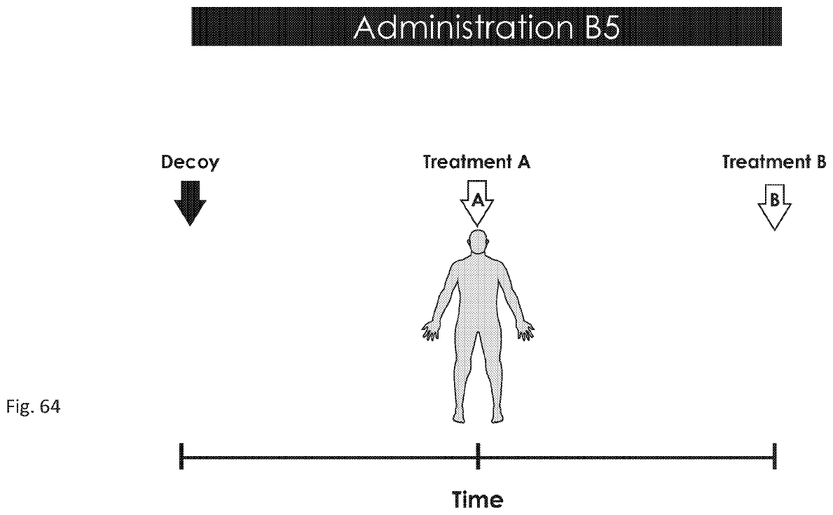

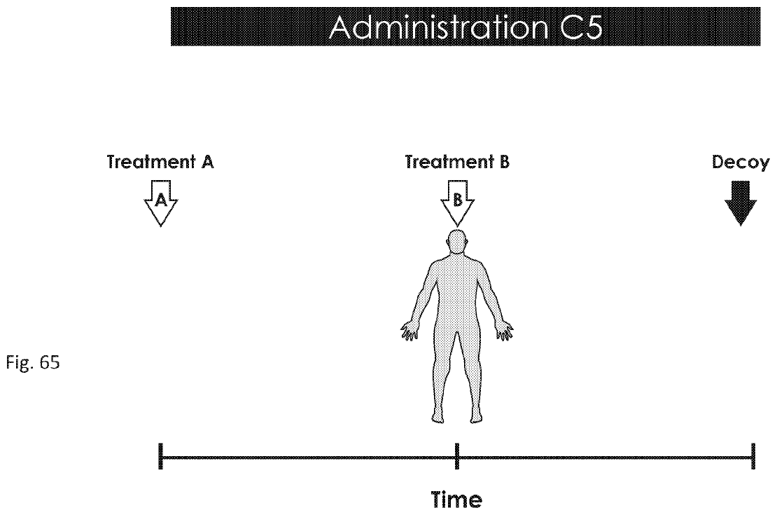

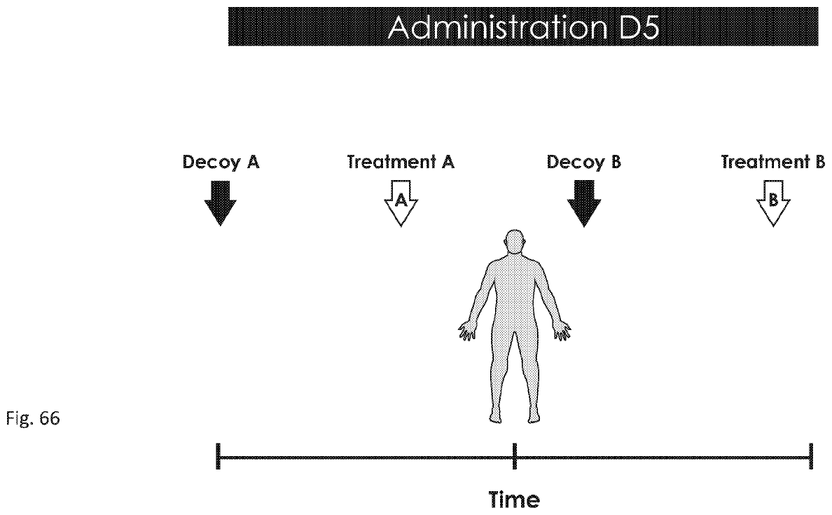

Any decoy product or therapy encompassed herein may be administered to an individual as a standalone treatment (FIG. 44). The decoy may be administered after (FIG. 45), or before (FIG. 46), or before and after (FIG. 48) a non-decoy treatment. A non-decoy treatment may be administered before and after the decoy is administered (FIG. 47). A decoy may be administered more than once to an individual (FIG. 49) including between administration of a non-decoy therapy (FIG. 50) or administered more than once before and after the administration of a non-decoy therapy (FIG. 51). A decoy may be administered concurrently with another treatment (FIG. 52). The decoy may be administered concurrently with a non-decoy treatment to an individual that is undergoing a dosing regimen of the non-decoy treatment (FIG. 53). The decoy may be administered concurrently with a non-decoy therapy to an individual that is undergoing a dosing regimen of the decoy (FIG. 54). The decoy therapy administered to the individual may comprise two non-decoy decoy compositions (FIG. 55), which may be administered in between administrations of a non-decoy treatment (FIG. 56) or before and after administration of a non-decoy treatment (FIG. 57). A first decoy therapy may be administered before administration of a non-decoy therapy followed by administration of a second decoy (FIG. 58). The first decoy and the second decoy may be administered more than once before or after the administration of the non-decoy therapy (FIG. 59). A first decoy, a second decoy, and a non-decoy therapy may be administered to an individual concurrently (FIG. 60). The first decoy, second decoy, and non-decoy therapy may be administered concurrently after a first administration of the first decoy or second decoy and before a second administration of the first decoy or second decoy (FIGS. 61-62). A first non-decoy treatment may be administered to an individual followed by a decoy followed by a second non-decoy treatment (FIG. 63). A decoy may be administered to an individual followed by a first non-decoy treatment followed by a second non-decoy treatment (FIG. 64). A first non-decoy may be administered to an individual followed by a second non-decoy treatment followed by a decoy treatment (FIG. 65). A first decoy may be administered to an individual followed by a first non-decoy treatment followed by a second decoy followed by a second non-decoy therapy (FIG. 66).

### REFERENCES

The following references, to the extent that they provide exemplary procedural or other details supplementary to those set forth herein, are specifically incorporated herein by reference.
Porter D L, Levine B L, Kalos M, Bagg A, & June C H (2011) Chimeric antigen receptor-modified T cells in chronic lymphoid leukemia. The New England journal of medicine 365(8):725-733.
Brentjens R J, Davila M L, Riviere I, Park J, Wang X, Cowell L G, Bartido S, Stefanski J, Taylor C, Olszewska M, Borquez-Ojeda 0, Qu J, Wasielewska T, He Q, Bernal Y, Rijo I V, Hedvat C, Kobos R, Curran K, Steinherz P, Jurcic J, Rosenblat T, Maslak P, Frattini M, & Sadelain M (2013) CD19-targeted T cells rapidly induce molecular remissions in adults with chemotherapy-refractory acute lymphoblastic leukemia. Science translational medicine 5(177):177ra138.
Ahmed N, Brawley V S, Hegde M, Robertson C, Ghazi A, Gerken C, Liu E, Dakhova 0, Ashoori A, Corder A, Gray T, Wu M F, Liu H, Hicks J, Rainusso N, Dotti G, Mei Z, Grilley B, Gee A, Rooney C M, Brenner M K, Heslop H E, Wels W S, Wang L L, Anderson P, & Gottschalk S (2015) Human Epidermal Growth Factor Receptor 2 (HER2) -Specific Chimeric Antigen Receptor-Modified T Cells for the Immunotherapy of HER2-Positive Sarcoma. J.Clin.Oncol. 33(15):1688-1696.
Morgan R A, Johnson L A, Davis J L, Zheng Z, Woolard K D, Reap E A, Feldman S A, Chinnasamy N, Kuan C T, Song H, Zhang W, Fine H A, & Rosenberg S A (2012) Recognition of glioma stem cells by genetically modified T cells targeting EGFRvIII and development of adoptive cell therapy for glioma. Human gene therapy 23(10):1043-1053.
Kershaw M H, Westwood J A, Parker L L, Wang G, Eshhar Z, Mavroukakis S A, White D E, Wunderlich J R, Canevari S, Rogers-Freezer L, Chen C C, Yang J C, Rosenberg S A, & Hwu P (2006) A phase I study on adoptive immunotherapy using gene-modified T cells for ovarian cancer. Clin.Cancer Res. 12(20 Pt 1):6106-6115.
Parker L L, Do M T, Westwood J A, Wunderlich J R, Dudley M E, Rosenberg S A, & Hwu P (2000) Expansion and characterization of T cells transduced with a chimeric receptor against ovarian cancer. Human gene therapy 11(17):2377-2387.
Pule M A, Savoldo B, Myers G D, Rossig C, Russell H V, Dotti G, Huls M H, Liu E, Gee A P, Mei Z, Yvon E, Weiss H L, Liu H, Rooney C M, Heslop H E, & Brenner M K (2008) Virus-specific T cells engineered to coexpress tumor-specific receptors: persistence and antitumor activity in individuals with neuroblastoma. Nat.Med. 14(11):1264-1270.
Louis C U, Savoldo B, Dotti G, Pule M, Yvon E, Myers G D, Rossig C, Russell H V, Diouf O, Liu E, Liu H, Wu M F, Gee A P, Mei Z, Rooney C M, Heslop H E, & Brenner M K (2011) Antitumor activity and long-term fate of chimeric antigen receptor-positive T cells in patients with neuroblastoma. Blood 118(23):6050-6056.
Scarfo I & Maus M V (2017) Current approaches to increase CAR T cell potency in solid tumors: targeting the tumor microenvironment. Journal for immunotherapy of cancer 5:28.
Newick K, O'Brien S, Moon E, & Albelda S M (2017) CAR T Cell Therapy for Solid Tumors. Annual review of medicine 68:139-152.
DeRenzo C & Gottschalk S (2019) Genetic Modification Strategies to Enhance CAR T Cell Persistence for Patients With Solid Tumors. Frontiers in immunology 10:218.
Martinez M & Moon E K (2019) CAR T Cells for Solid Tumors: New Strategies for Finding, Infiltrating, and Surviving in the Tumor Microenvironment. Frontiers in immunology 10:128.
Coley W B (1991) The treatment of malignant tumors by repeated inoculations of erysipelas. With a report of ten original cases. 1893. Clin Orthop Relat Res (262):3-11.
Kienle G S (2012) Fever in Cancer Treatment: Coley's Therapy and Epidemiologic Observations. Glob Adv Health Med 1(1):92-100.
McCarthy E F (2006) The toxins of William B. Coley and the treatment of bone and soft-tissue sarcomas. The Iowa orthopaedic journal 26:154-158.
Miller J F (1959) Role of the thymus in murine leukaemia. Nature 183(4667):1069.
Miller J F, Mitchell G F, & Weiss N S (1967) Cellular basis of the immunological defects in thymectomized mice. Nature 214(5092):992-997.
Cooper M D, Peterson R D, & Good R A (1965) Delineation of the Thymic and Bursal Lymphoid Systems in the Chicken. Nature 205:143-146.
Steinman R M & Cohn Z A (2007) Pillars Article: Identification of a novel cell type in peripheral lymphoid organs of mice. I. Morphology, quantitation, tissue distribution. J. Exp. Med.1973. 137: 1142-1162. Journal of immunology 178(1):5-25.
   Kiessling R, Klein E, Pross H, & Wigzell H (1975) “Natural” killer cells in the mouse. II. Cytotoxic cells with specificity for mouse Moloney leukemia cells. Characteristics of the killer cell. European journal of immunology 5(2):117-121.
Kiessling R, Klein E, & Wigzell H (1975) “Natural” killer cells in the mouse. I. Cytotoxic cells with specificity for mouse Moloney leukemia cells. Specificity and distribution according to genotype. European journal of immunology 5(2):112-117.
Melief C J, van Hall T, Arens R, Ossendorp F, & van der Burg S H (2015) Therapeutic cancer vaccines. The Journal of clinical investigation 125(9):3401-3412.
Banchereau J & Palucka K (2018) Immunotherapy: Cancer vaccines on the move. Nature reviews. Clinical oncology 15(1):9-10.
Kalinski P, Urban J, Narang R, Berk E, Wieckowski E, & Muthuswamy R (2009) Dendritic cell-based therapeutic cancer vaccines: what we have and what we need. Future oncology 5(3):379-390.
Lutz E, Yeo C J, Lillemoe K D, Biedrzycki B, Kobrin B, Herman J, Sugar E, Piantadosi S, Cameron J L, Solt S, Onners B, Tartakovsky I, Choi M, Sharma R, Illei P B,
Hruban R H, Abrams R A, Le D, Jaffee E, & Laheru D (2011) A lethally irradiated allogeneic granulocyte-macrophage colony stimulating factor-secreting tumor vaccine for pancreatic adenocarcinoma. A Phase II trial of safety, efficacy, and immune activation. Annals of surgery 253(2):328-335.
Kantoff P W, Higano C S, Shore N D, Berger E R, Small E J, Penson D F, Redfern C H, Ferrari A C, Dreicer R, Sims R B, Xu Y, Frohlich M W, Schellhammer P F, & Investigators IS (2010) Sipuleucel-T immunotherapy for castration-resistant prostate cancer. The New England journal of medicine 363(5):411-422.
Kalinski P, Muthuswamy R, & Urban J (2013) Dendritic cells in cancer immunotherapy: vaccines and combination immunotherapies. Expert review of vaccines 12(3):285-295.
Andersen M H, Junker N, Ellebaek E, Svane I M, & Thor Straten P (2010) Therapeutic cancer vaccines in combination with conventional therapy. Journal of biomedicine & biotechnology 2010:237623.
Weiner L M, Murray J C, & Shuptrine C W (2012) Antibody-based immunotherapy of cancer. Cell 148(6):1081-1084.
Weiner L M, Surana R, & Wang S (2010) Monoclonal antibodies: versatile platforms for cancer immunotherapy. Nature reviews. Immunology 10(5):317-327.
Weiner G J (2010) Rituximab: mechanism of action. Seminars in hematology 47(2):115-123.
Jonker D J, O'Callaghan C J, Karapetis C S, Zalcberg J R, Tu D, Au H J, Berry S R, Krahn M, Price T, Simes R J, Tebbutt N C, van Hazel G, Wierzbicki R, Langer C, & Moore M J (2007) Cetuximab for the treatment of colorectal cancer. The New England journal of medicine 357(20):2040-2048.
Mazzarella L, Guida A, & Curigliano G (2018) Cetuximab for treating non-small cell lung cancer. Expert Opin Biol Ther 18(4):483-493.
Gunturu K S, Woo Y, Beaubier N, Remotti H E, & Saif M W (2013) Gastric cancer and trastuzumab: first biologic therapy in gastric cancer. Ther Adv Med Oncol 5(2):143-151.
Maximiano S, Magalhaes P, Guerreiro M P, & Morgado M (2016) Trastuzumab in the Treatment of Breast Cancer. BioDrugs 30(2):75-86.
Valabrega G, Montemurro F, & Aglietta M (2007) Trastuzumab: mechanism of action, resistance and future perspectives in HER2-overexpressing breast cancer. Annals of oncology: official journal of the European Society for Medical Oncology 18(6):977-984.
Azoury S C, Straughan D M, & Shukla V (2015) Immune Checkpoint Inhibitors for Cancer Therapy: Clinical Efficacy and Safety. Current cancer drug targets 15(6):452-462.
Marin-Acevedo J A, Dholaria B, Soyano A E, Knutson K L, Chumsri S, & Lou Y (2018) Next generation of immune checkpoint therapy in cancer: new developments and challenges. J Hematol Oncol 11(1):39.
Postow M A, Callahan M K, & Wolchok J D (2015) Immune Checkpoint Blockade in Cancer Therapy. Journal of clinical oncology: official journal of the American Society of Clinical Oncology 33(17):1974-1982.
Wei S C, Duffy C R, & Allison J P (2018) Fundamental Mechanisms of Immune Checkpoint Blockade Therapy. Cancer discovery 8(9):1069-1086.
Robert C, Long G V, Brady B, Dutriaux C, Maio M, Mortier L, Hassel J C, Rutkowski P, McNeil C, Kalinka-Warzocha E, Savage K J, Hernberg M M, Lebbe C, Charles J, Mihalcioiu C, Chiarion-Sileni V, Mauch C, Cognetti F, Arance A, Schmidt H, Schadendorf D, Gogas H, Lundgren-Eriksson L, Horak C, Sharkey B, Waxman I M, Atkinson V, & Ascierto P A (2015) Nivolumab in previously untreated melanoma without BRAF mutation. The New England journal of medicine 372(4):320-330.
Larkin J, Chiarion-Sileni V, Gonzalez R, Grob J J, Cowey C L, Lao C D, Schadendorf D, Dummer R, Smylie M, Rutkowski P, Ferrucci P F, Hill A, Wagstaff J, Carlino M S, Haanen J B, Maio M, Marquez-Rodas I, McArthur G A, Ascierto P A, Long G V, Callahan M K, Postow M A, Grossmann K, Sznol M, Dreno B, Bastholt L, Yang A, Rollin L M, Horak C, Hodi F S, & Wolchok J D (2015) Combined Nivolumab and Ipilimumab or Monotherapy in Untreated Melanoma. The New England journal of medicine 373(1):23-34.
Kantarjian H, Stein A, Gokbuget N, Fielding A K, Schuh A C, Ribera J M, Wei A, Dombret H, Foa R, Bassan R, Arslan O, Sanz M A, Bergeron J, Demirkan F, Lech-Maranda E, Rambaldi A, Thomas X, Horst H A, Bruggemann M, Klapper W, Wood B L, Fleishman A, Nagorsen D, Holland C, Zimmerman Z, & Topp M S (2017) Blinatumomab versus Chemotherapy for Advanced Acute Lymphoblastic Leukemia. The New England journal of medicine 376(9):836-847.
Glassman P M & Balthasar J P (2014) Mechanistic considerations for the use of monoclonal antibodies for cancer therapy. Cancer biology & medicine 11(1):20-33.
Davis T A, White C A, Grillo-Lopez A J, Velasquez W S, Link B, Maloney D G, Dillman R O, Williams M E, Mohrbacher A, Weaver R, Dowden S, & Levy R (1999) Single-agent monoclonal antibody efficacy in bulky non-Hodgkin's lymphoma: results of a phase II trial of rituximab. Journal of clinical oncology: official journal of the American Society of Clinical Oncology 17(6):1851-1857.
Kolb H J, Mittermuller J, Clemm C, Holler E, Ledderose G, Brehm G, Heim M, & Wilmanns W (1990) Donor leukocyte transfusions for treatment of recurrent chronic myelogenous leukemia in marrow transplant patients. Blood 76(12):2462-2465.
Lokhorst H M, Schattenberg A, Cornelissen J J, Thomas L L, & Verdonck L F (1997) Donor leukocyte infusions are effective in relapsed multiple myeloma after allogeneic bone marrow transplantation. Blood 90(10):4206-4211.
Slavin S, Naparstek E, Nagler A, Ackerstein A, Samuel S, Kapelushnik J, Brautbar C, & Or R (1996) Allogeneic cell therapy with donor peripheral blood cells and recombinant human interleukin-2 to treat leukemia relapse after allogeneic bone marrow transplantation. Blood 87(6):2195-2204.
Frey N V & Porter D L (2008) Graft-versus-host disease after donor leukocyte infusions: presentation and management. Best Pract Res Clin Haematol 21(2):205-222.
Dudley M E & Rosenberg S A (2007) Adoptive cell transfer therapy. Seminars in oncology 34(6):524-531.
Rosenberg S A & Dudley M E (2009) Adoptive cell therapy for the treatment of patients with metastatic melanoma. Current opinion in immunology 21(2):233-240.
Dudley M E, Yang J C, Sherry R, Hughes M S, Royal R, Kammula U, Robbins P F, Huang J, Citrin D E, Leitman S F, Wunderlich J, Restifo N P, Thomasian A, Downey S G, Smith F O, Klapper J, Morton K, Laurencot C, White D E, & Rosenberg S A (2008) Adoptive cell therapy for patients with metastatic melanoma: evaluation of intensive myeloablative chemoradiation preparative regimens. Journal of clinical oncology: official journal of the American Society of Clinical Oncology 26(32):5233-5239.
Bollard C M, Gottschalk S, Leen A M, Weiss H, Straathof K C, Carrum G, Khalil M, Wu M F, Huls M H, Chang C C, Gresik M V, Gee A P, Brenner M K, Rooney C M, & Heslop H E (2007) Complete responses of relapsed lymphoma following genetic modification of tumor-antigen presenting cells and T-lymphocyte transfer. Blood 110(8):2838-2845.
Bollard C M, Gottschalk S, Torrano V, Diouf O, Ku S, Hazrat Y, Carrum G, Ramos C, Fayad L, Shpall E J, Pro B, Liu H, Wu M F, Lee D, Sheehan A M, Zu Y, Gee A P, Brenner M K, Heslop H E, & Rooney C M (2014) Sustained complete responses in patients with lymphoma receiving autologous cytotoxic T lymphocytes targeting Epstein-Barr virus latent membrane proteins. J.Clin.Oncol. 32(8):798-808.
Johnson L A, Heemskerk B, Powell D J, Jr., Cohen C J, Morgan R A, Dudley M E, Robbins P F, & Rosenberg S A (2006) Gene transfer of tumor-reactive TCR confers both high avidity and tumor reactivity to nonreactive peripheral blood mononuclear cells and tumor-infiltrating lymphocytes. Journal of immunology 177(9):6548-6559.
Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, & Rosenberg S A (2006) Cancer regression in patients after transfer of genetically engineered lymphocytes. Science 314(5796):126-129.
Johnson L A, Morgan R A, Dudley M E, Cassard L, Yang J C, Hughes M S, Kammula U S, Royal R E, Sherry R M, Wunderlich J R, Lee C C, Restifo N P, Schwarz S L, Cogdill A P, Bishop R J, Kim H, Brewer C C, Rudy S F, VanWaes C, Davis J L, Mathur A, Ripley R T, Nathan D A, Laurencot C M, & Rosenberg S A (2009) Gene therapy with human and mouse T-cell receptors mediates cancer regression and targets normal tissues expressing cognate antigen. Blood 114(3):535-546.
Wang M, Yin B, Wang H Y, & Wang R F (2014) Current advances in T-cell-based cancer immunotherapy. Immunotherapy 6(12):1265-1278.
Robbins P F, Li Y F, El-Gamil M, Zhao Y, Wargo J A, Zheng Z, Xu H, Morgan R A, Feldman S A, Johnson L A, Bennett A D, Dunn S M, Mahon™, Jakobsen B K, & Rosenberg S A (2008) Single and dual amino acid substitutions in TCR CDRs can enhance antigen-specific T cell functions. Journal of immunology 180(9):6116-6131.
Robbins P F, Kassim S H, Tran T L, Crystal J S, Morgan R A, Feldman S A, Yang J C, Dudley M E, Wunderlich J R, Sherry R M, Kammula U S, Hughes M S, Restifo N P, Raffeld M, Lee C C, Li Y F, El-Gamil M, & Rosenberg S A (2015) A pilot trial using lymphocytes genetically engineered with an N Y-ESO-1-reactive T-cell receptor: long-term follow-up and correlates with response. Clinical cancer research: an official journal of the American Association for Cancer Research 21(5):1019-1027.
Rapoport A P, Stadtmauer E A, Binder-Scholl G K, Goloubeva 0, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin H K, Ribeiro L, Holdich T, Yanovich S, Hardy N, Yared J, Kerr N, Philip S, Westphal S, Siegel D L, Levine B L, Jakobsen B K, Kalos M, & June C H (2015) N Y-ESO-1-specific TCR-engineered T cells mediate sustained antigen-specific antitumor effects in myeloma. Nature medicine 21(8):914-921.
Morgan R A, Chinnasamy N, Abate-Daga D, Gros A, Robbins P F, Zheng Z, Dudley M E, Feldman S A, Yang J C, Sherry R M, Phan G Q, Hughes M S, Kammula U S, Miller A D, Hessman C J, Stewart A A, Restifo N P, Quezado M M, Alimchandani M, Rosenberg A Z, Nath A, Wang T, Bielekova B, Wuest S C, Akula N, McMahon F J, Wilde S, Mosetter B, Schendel D J, Laurencot C M, & Rosenberg S A (2013) Cancer regression and neurological toxicity following anti-MAGE-A3 TCR gene therapy. J Immunother 36(2):133-151.
Linette G P, Stadtmauer E A, Maus M V, Rapoport A P, Levine B L, Emery L, Litzky L, Bagg A, Carreno B M, Cimino P J, Binder-Scholl G K, Smethurst D P, Gerry A B, Pumphrey N J, Bennett A D, Brewer J E, Dukes J, Harper J, Tayton-Martin H K, Jakobsen B K, Hassan N J, Kalos M, & June C H (2013) Cardiovascular toxicity and titin cross-reactivity of affinity-enhanced T cells in myeloma and melanoma. Blood 122(6):863-871.
Bendle G M, Linnemann C, Hooijkaas A I, Bies L, de Witte M A, Jorritsma A, Kaiser A D, Pouw N, Debets R, Kieback E, Uckert W, Song J Y, Haanen J B, & Schumacher T N (2010) Lethal graft-versus-host disease in mouse models of T cell receptor gene therapy. Nature medicine 16(5):565-570, 561p following 570.
Reuss S, Sebestyen Z, Heinz N, Loew R, Baum C, Debets R, & Uckert W (2014) TCR-engineered T cells: a model of inducible TCR expression to dissect the interrelationship between two TCRs. European journal of immunology 44(1):265-274.
Bunse M, Bendle G M, Linnemann C, Bies L, Schulz S, Schumacher T N, & Uckert W (2014) RNAi-mediated TCR knockdown prevents autoimmunity in mice caused by mixed TCR dimers following TCR gene transfer. Molecular therapy: the journal of the American Society of Gene Therapy 22(11):1983-1991.
Okamoto S, Amaishi Y, Goto Y, Ikeda H, Fujiwara H, Kuzushima K, Yasukawa M, Shiku H, & Mineno J (2012) A Promising Vector for TCR Gene Therapy: Differential Effect of siRNA, 2A Peptide, and Disulfide Bond on the Introduced TCR Expression. Mol Ther Nucleic Acids 1:e63.
Okamoto S, Mineno J, Ikeda H, Fujiwara H, Yasukawa M, Shiku H, & Kato I (2009) Improved expression and reactivity of transduced tumor-specific TCRs in human lymphocytes by specific silencing of endogenous TCR. Cancer research 69(23):9003-9011.
Garrido F, Aptsiauri N, Doorduijn E M, Garcia Lora A M, & van Hall T (2016) The urgent need to recover MHC class I in cancers for effective immunotherapy. Current opinion in immunology 39:44-51.
Eshhar Z, Waks T, Gross G, & Schindler D G (1993) Specific activation and targeting of cytotoxic lymphocytes through chimeric single chains consisting of antibody-binding domains and the gamma or zeta subunits of the immunoglobulin and T-cell receptors. Proceedings of the National Academy of Sciences of the United States of America 90(2):720-724.
Gross G, Waks T, & Eshhar Z (1989) Expression of immunoglobulin-T-cell receptor chimeric molecules as functional receptors with antibody-type specificity. Proceedings of the National Academy of Sciences of the United States of America 86(24):10024-10028.
Eshhar Z (2014) From the mouse cage to human therapy: a personal perspective of the emergence of T-bodies/chimeric antigen receptor T cells. Human gene therapy 25(9):773-778.
Lamers C H, Sleijfer S, Vulto A G, Kruit W H, Kliffen M, Debets R, Gratama J W, Stoter G, & Oosterwijk E (2006) Treatment of metastatic renal cell carcinoma with autologous T-lymphocytes genetically retargeted against carbonic anhydrase IX: first clinical experience. J.Clin.Oncol. 24(13):e20-e22.
Park J R, Digiusto D L, Slovak M, Wright C, Naranjo A, Wagner J, Meechoovet H B, Bautista C, Chang W C, Ostberg J R, & Jensen M C (2007) Adoptive transfer of chimeric antigen receptor re-directed cytolytic T lymphocyte clones in patients with neuroblastoma. Mol.Ther. 15(4):825-833.
Gross G & Eshhar Z (2016) Therapeutic Potential of T Cell Chimeric Antigen Receptors (CARs) in Cancer Treatment: Counteracting Off-Tumor Toxicities for Safe CAR T Cell Therapy. Annual review of pharmacology and toxicology 56:59-83.
Kershaw M H, Westwood J A, & Darcy P K (2013) Gene-engineered T cells for cancer therapy. Nature reviews. Cancer 13(8):525-541.
Brentjens R J, Davila M L, Riviere I, Park J, Wang X, Cowell L G, Bartido S, Stefanski J, Taylor C, Olszewska M, Borquez-Ojeda 0, Qu J, Wasielewska T, He Q, Bernal Y, Rijo I V, Hedvat C, Kobos R, Curran K, Steinherz P, Jurcic J, Rosenblat T, Maslak P, Frattini M, & Sadelain M (2013) CD19-targeted T cells rapidly induce molecular remissions in adults with chemotherapy-refractory acute lymphoblastic leukemia. Sci.Transl.Med. 5(177):177ra138.
Grupp S A, Kalos M, Barrett D, Aplenc R, Porter D L, Rheingold S R, Teachey D T, Chew A, Hauck B, Wright J F, Milone M C, Levine B L, & June C H (2013) Chimeric antigen receptor-modified T cells for acute lymphoid leukemia. N.Engl.J.Med. 368(16):1509-1518.
Maude S L, Frey N, Shaw P A, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter D L, & Grupp S A (2014) Chimeric antigen receptor T cells for sustained remissions in leukemia. N.Engl.J.Med. 371(16):1507-1517.
Ruella M & Maus M V (2016) Catch me if you can: Leukemia Escape after CD19-Directed T Cell Immunotherapies. Comput Struct Biotechnol J 14:357-362.
Majzner R G & Mackall C L (2018) Tumor Antigen Escape from CAR T-cell Therapy. Cancer discovery 8(10):1219-1226.
Bonifant C L, Jackson H J, Brentjens R J, & Curran K J (2016) Toxicity and management in CAR T-cell therapy. Molecular therapy oncolytics 3:16011.
Minagawa K, Zhou X, Mineishi S, & Di Stasi A (2015) Seatbelts in CAR therapy: How Safe Are CARS? Pharmaceuticals 8(2):230-249.
Fitzgerald J C, Weiss S L, Maude S L, Barrett D M, Lacey S F, Melenhorst J J, Shaw P, Berg R A, June C H, Porter D L, Frey N V, Grupp S A, & Teachey D T (2017) Cytokine Release Syndrome After Chimeric Antigen Receptor T Cell Therapy for Acute Lymphoblastic Leukemia. Critical care medicine 45(2):e124-e131.
Hay K A, Hanafi L A, Li D, Gust J, Liles W C, Wurfel M M, Lopez J A, Chen J, Chung D, Harju-Baker S, Cherian S, Chen X, Riddell S R, Maloney D G, & Turtle C J (2017) Kinetics and biomarkers of severe cytokine release syndrome after CD19 chimeric antigen receptor-modified T-cell therapy. Blood 130(21):2295-2306.
Ruella M, Xu J, Barrett D M, Fraietta J A, Reich T J, Ambrose D E, Klichinsky M, Shestova 0, Patel P R, Kulikovskaya I, Nazimuddin F, Bhoj V G, Orlando E J, Fry T J, Bitter H, Maude S L, Levine B L, Nobles C L, Bushman F D, Young R M, Scholler J, Gill S I, June C H, Grupp S A, Lacey S F, & Melenhorst J J (2018) Induction of resistance to chimeric antigen receptor T cell therapy by transduction of a single leukemic B cell. Nature medicine 24(10):1499-1503.
Yoon D H, Osborn M J, Tolar J, & Kim C J (2018) Incorporation of Immune Checkpoint Blockade into Chimeric Antigen Receptor T Cells (CAR-Ts): Combination or Built-In CAR-T. Int J Mol Sci 19(2).
Maude S L, Barrett D, Teachey D T, & Grupp S A (2014) Managing cytokine release syndrome associated with novel T cell-engaging therapies. Cancer journal 20(2):119-122.
Blumenthal G M & Pazdur R (2018) Approvals in 2017: gene therapies and site-agnostic indications. Nature reviews. Clinical oncology.
Adusumilli P S, Cherkassky L, Villena-Vargas J, Colovos C, Servais E, Plotkin J, Jones D R, & Sadelain M (2014) Regional delivery of mesothelin-targeted CAR T cell therapy generates potent and long-lasting CD4-dependent tumor immunity. Science translational medicine 6(261):261ra151.
Beatty G L, Haas A R, Maus M V, Torigian D A, Soulen M C, Plesa G, Chew A, Zhao Y, Levine B L, Albelda S M, Kalos M, & June C H (2014) Mesothelin-specific chimeric antigen receptor mRNA-engineered T cells induce anti-tumor activity in solid malignancies. Cancer Immunol.Res. 2(2):112-120.
Schmidts A & Maus M V (2018) Making CAR T Cells a Solid Option for Solid Tumors. Frontiers in immunology 9:2593.
Ali A1, Oliver A J, Samiei T, Chan J D, Kershaw M H, & Slaney C Y (2019) Genetic Redirection of T Cells for the Treatment of Pancreatic Cancer. Front Oncol 9:56.
Jiang Z, Jiang X, Chen S, Lai Y, Wei X, Li B, Lin S, Wang S, Wu Q, Liang Q, Liu Q, Peng M, Yu F, Weng J, Du X, Pei D, Liu P, Yao Y, Xue P, & Li P (2016) Anti-GPC3-CAR T Cells Suppress the Growth of Tumor Cells in Patient-Derived Xenografts of Hepatocellular Carcinoma. Frontiers in immunology 7:690.
Wang L C, Lo A, Scholler J, Sun J, Majumdar R S, Kapoor V, Antzis M, Cotner C E, Johnson L A, Durham A C, Solomides C C, June C H, Pure E, & Albelda S M (2014) Targeting fibroblast activation protein in tumor stroma with chimeric antigen receptor T cells can inhibit tumor growth and augment host immunity without severe toxicity. Cancer immunology research 2(2):154-166.
Katz S C, Burga R A, McCormack E, Wang L J, Mooring W, Point G R, Khare P D, Thorn M, Ma Q, Stainken B F, Assanah E O, Davies R, Espat N J, & Junghans R P (2015) Phase I Hepatic Immunotherapy for Metastases Study of Intra-Arterial Chimeric Antigen Receptor-Modified T-cell Therapy for CEA+ Liver Metastases. Clinical cancer research: an official journal of the American Association for Cancer Research 21(14):3149-3159.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A soluble recombinant protein comprising (1) at least one inhibitory protein domain, wherein the inhibitory protein domain can bind to at least one immunosuppressive ligand, and (2) at least one activating protein domain.

2. The recombinant protein of claim 1, wherein the inhibitory protein domain comprises an extracellular domain selected from the group consisting of TGFBR2, FAS, IL4R, IL10R, and a combination thereof.

3. The recombinant protein of claim 1 or 2, wherein the immunosuppressive ligand is selected from the group consisting of TGF-β, FASL, IL4, IL10, or a combination thereof

4. The recombinant protein of any one of claims 1-3, wherein the inhibitory protein domain comprises one or more mutations from a natural protein sequence of the inhibitory protein domain.

5. The recombinant protein of any one of claims 1-4, wherein the activating protein domain comprises at least one domain selected from the group consisting of IL-2, IL-7, IL-15, and a combination thereof.

6. The recombinant protein of any one of claims 1-5, wherein at least one inhibitory protein domain is linked to at least one activating protein domain via a protein linker.

7. The recombinant protein of claim 6, wherein the protein linker comprises a G-S linker.

8. The recombinant protein of claim 7, wherein the G-S linker comprises the protein sequence comprising GGGSGGGGSGGGGSGGG (SEQ ID NO:1).

9. The recombinant protein of any one of claims 1-8, wherein the protein comprises:
an extracellular domain of TGFBR2 and IL-2;
an extracellular domain of TGFBR2 and IL-7;
an extracellular domain of TGFBR2 and IL-15;
an extracellular domain of FAS and IL-2;
an extracellular domain of FAS and IL-7;
an extracellular domain of FAS and IL-15;
an extracellular domain of IL4R and IL-7;
an extracellular domain of IL4R and IL-15;
an extracellular domain of IL4R and IL-2 an extracellular domain of IL10R and IL-2;
an extracellular domain of IL10R and IL-7; or
an extracellular domain of IL10R and IL-15.

10. A nucleic acid comprising a sequence encoding the recombinant protein of any one of claims 1-9.

11. A vector comprising a nucleic acid sequence encoding the recombinant protein of any one of claims 1-9.

12. The vector of claim 11, wherein the vector comprises a viral vector or a non-viral vector.

13. The vector of claim 11 or 12, wherein the genetic vector comprises a transient expression vector or a stable expression vector.

14. A cell comprising the recombinant protein of any one of claims 1-9 and/or the vector of any one of claims 11-13.

15. The cell of claim 14, wherein the cell is a T lymphocyte, a natural killer cell, a macrophage, a mesenchymal stromal cell, tumor infiltrating cell, NK cell, NK T cell, or a fibroblast.

16. The cell of claim 14, wherein the cell is a T lymphocyte.

17. The cell of claim 14, wherein the cell is an NK cell.

18. The cell of any one of claims 15-17, wherein the cell is genetically modified to express at least one additional recombinant protein.

19. The cell of claim 18, wherein the additional recombinant protein comprises an engineered antigen receptor or an antibody.

20. The cell of claim 19, wherein the engineered receptor comprises a chimeric antigen receptor or a transgenic T cell receptor.

21. The cell of any one of claims 16-20, wherein the cell is a T cell, NK cell, or NK T cell comprising one or more chimeric antigen receptors or one or more transgenic T cell receptors.

22. The cell of any one of claims 16-21, wherein the cell is a tumor-specific T cell generated by ex vivo antigen/peptide stimulation.

23. The cell of any one of claims 16-22, wherein the cell comprises 1, 2, or more of the following:
an extracellular domain of TGFBR2 and IL-2;
an extracellular domain of TGFBR2 and IL-7;
an extracellular domain of TGFBR2 and IL-15;
an extracellular domain of FAS and IL-2;
an extracellular domain of FAS and IL-7;
an extracellular domain of FAS and IL-15;
an extracellular domain of IL4R and IL-2;
an extracellular domain of IL4R and IL-7;
an extracellular domain of IL4R and IL-15;
an extracellular domain of IL10R and IL-2;
an extracellular domain of IL10R and IL-7; or
an extracellular domain of IL10R and IL-15.

24. A method of treating an individual comprising administering a therapeutically effective amount of the recombinant protein of any one of claims 1-11 and/or a therapeutically effective amount of the cells of any one of claims 16-23.

25. The method of claim 24, wherein the method comprises administering a therapeutically effective amount of the cells of any one of claims 16-23 and comprises administering a therapeutically effective amount of cells comprising anti-cancer activity.

26. The method of claim 25, wherein the cells of any one of claims 16-23 that are autologous with respect to the individual and are further defined as immune cells engineered to express the protein.

27. The method of claim 25, wherein the cells of any one of claims 16-23 that are allogeneic with respect to the individual and are further defined as immune cells engineered to express the protein.

28. The method of claim 26 or 27, wherein the immune cells are T lymphocyte, a natural killer cell, a macrophage, a mesenchymal stromal cell, tumor infiltrating cell, NK cell, or NK T cells.

29. The method of claim 25, wherein the cells of any one of claims 16-23 that are autologous with respect to the individual and are further defined as virus-specific T cells.

30. The method of claim 25, wherein the cells of any one of claims 16-23 that are allogeneic with respect to the individual and are further defined as virus-specific T cells.

31. The method of any one of claims 25-30, wherein the cells comprising anti-cancer activity comprise one or more engineered antigen receptors or one or more antibodies, any of which target a cancer antigen.

32. The method of any one of claims 24-31, wherein the individual has or is suspected of having cancer.

33. The method of any one of claims 24-32, wherein the therapeutically effective amount comprises a single dose or multiple doses.

34. The method of any one of claims 24-33, further comprising administering a therapeutically effective amount of one or more additional therapeutic compositions to the individual.

35. The method of claim 34, wherein the additional therapeutic composition can activate immune responses directly or indirectly.

36. The method of any one of claims 24-35, further comprising the step of selecting the recombinant protein based on a cancer type of the individual.


SEQUENCE LISTING (13 sequences)

SEQ ID NO: 1 - protein, 17 aa, synthetic construct
GGGSGGGGSGGGGSGGG

SEQ ID NO: 2 - protein, 173 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 3 - DNA, 522 bp, synthetic construct
  source 1..522: mol_type = other DNA
atgttaggtatctggactctgctgcctctagtgttgacctctgtcgctagactgagcagtaaatcagtcaacgctcaggtgactgatattaactccaaagggctggagcttcggaaaacggtgacaacagttgagacccagaacctggagggacttcaccacgacggtcagttttgccacaaaccttgtccccctggcgaacgaaaagcacgagattgcactgtcaacggagacgagccagactgcgtcccctgtcaggagggcaaagagtacactgataaggcccacttttcctcaaagtgccgccgctgtaggctgtgtgacgaaggccacgggctcgaagttgaaatcaattgcacgagaacacagaacactaagtgtcgttgcaagccgaatttcttttgtaacagcacagtctgcgagcactgcgacccttgtacgaagtgtgagcacgggattattaaggagtgtaccctaacctccaataccaagtgcaaggaagaaggaagcaggagcaactga

SEQ ID NO: 4 - protein, 173 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCSRCRLCDEGHGLEVEINCTRTQNTKCRCKPNFFCNSTVCEHCDPCTKCEHGIIKECTLTSNTKCKEEGSRSN

SEQ ID NO: 5 - DNA, 522 bp, synthetic construct
  source 1..522: mol_type = other DNA
atgttaggtatctggactctgctgcctctagtgttgacctctgtcgctagactgagcagtaaatcagtcaacgctcaggtgactgatattaactccaaagggctggagcttcggaaaacggtgacaacagttgagacccagaacctggagggacttcaccacgacggtcagttttgccacaaaccttgtccccctggcgaacgaaaagcacgagattgcactgtcaacggagacgagccagactgcgtcccctgtcaggagggcaaagagtacactgataaggcccacttttcctcaaagtgctctcgctgtaggctgtgtgacgaaggccacgggctcgaagttgaaatcaattgcacgagaacacagaacactaagtgtcgttgcaagccgaatttcttttgtaacagcacagtctgcgagcactgcgacccttgtacgaagtgtgagcacgggattattaaggagtgtaccctaacctccaataccaagtgcaaggaagaaggaagcaggagcaactga

SEQ ID NO: 6 - protein, 631 aa, synthetic construct
MKWVTFLLLLFISGSAFSAAANGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 7 - DNA, 1896 bp, synthetic construct
  source 1..1896: mol_type = other DNA
atgaaatgggtgacatttctgctgctgctgtttatctccggttccgctttttccgccgctgccaacggggccgtgaaatttccacagctgtgcaagttttgtgacgtacggttttccacctgcgacaaccaaaagtcttgtatgtctaattgcagtatcacctccatttgtgagaagccacaggaggtctgcgttgccgtatggcggaagaatgacgaaaacattacacttgaaaccgtgtgccacgaccctaaacttccataccatgacttcatcctggaagatgccgctagtccgaagtgcattatgaaagagaagaaaaagcccggcgagacattcttcatgtgttcctgttcttctgatgagtgcaacgacaacatcatcttcagtgaggaatataacacaagcaaccctgacggactgggccccgtggaaagttccccaggccacggcttagacacagcagctgcaggtcctgagcccagtactcgatgcgagcttagtcccattaatgcttctcaccccgtccaagcactcatggagtctttcacagtactcagcggctgcgcatcgcacggcactaccggactgcccagggaggtccatgttttgaatctccgctcaactgatcagggcccaggccagaggcagcgagaggtgactctccatctgaatcccatcgcaagcgtccatacacaccacaagccaatagttttcctgttaaactctccacagccactagtatggagactcaagaccgagagactggctgccggggtgcctagacttttcctggtcagtgaaggttccgtggtccagtttccttccggcaacttcagcctgactgctgagaccgaagagcgtaacttcccacaagagaatgaacatttgctgagatgggctcaaaaggaatacggcgcagtaacctcctttacggagctgaagatcgcgcgcaacatctatattaaagtaggcgaggaccaagtctttcctccaacctgtaacatcggaaagaactttctgtcgctgaactatctggccgaatatctgcaacctaaagccgctgaggggtgcgttttaccatctcagccccatgaaaaggaggtacatatcatagagttaataacccccagtagcaacccatattccgcctttcaggttgacataatcgtcgatattagaccagcccaggaagaccccgaagtggtcaaaaaccttgtactcatcttgaaatcaaagaagagcgtgaattgggtgatcaaaagcttcgacgttaaagggaacctgaaagtgattgctcctaacagcatcggcttcgggaaggagtcggagcggagtatgacgatgacgaaactagtgcgggatgatattccatcaacgcaagagaacttaatgaaatgggcactggatgcaggttatagaccagtgaccagctataccatggcccccgtagccaatagattccacctaaggctagaaaacaacgaggaaatgcgcgacgaggaggtccatacaatccctccagagctgaggatcttgttggacccagacaagctgccacagttgtgtaagttctgtgatgtccgcttttcaacatgcgataaccagaagagctgcatgtcaaattgcagtattacatcaatttgtgaaaagccccaggaggtatgtgtggccgtttggaggaaaaacgatgagaacataacattggagacggtgtgtcatgatcccaaactgccgtatcatgatttcatactggaggatgccgcctcccccaaatgcatcatgaaggaaaagaaaaagcctggagaaacttttttcatgtgcagctgctcaagtgacgaatgcaatgataacattatcttcagcgaagaatataacacgtcaaatcctgactag

SEQ ID NO: 8 - protein, 365 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 9 - DNA, 1098 bp, synthetic construct
  source 1..1098: mol_type = other DNA
atgggtcgaggacttctaagaggtctgtggccacttcatattgttctgtggacgcgtatcgcttctacaataccacctcacgttcagaagtcagttaataacgacatgattgtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaaaccacaactcctgcgccacggcctccgacgcccgctcctacgatagccagtcagcccttgtcattgcgacccgaagcctgtcggcctgcagctggaggtgcagtgcacacgagggggctggacttcgcctgtgatatctacatctgggcgcccttggccgggacttgtggggtccttctcctgtcactggttatcaccctttactgcagagtgaagttcagcaggagcgcagacgcccccgcgtaccagcagggccagaaccagctctataacgagctcaatctaggacggagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtctacttacatgcggtgatgtggaagagaatcccggcccttag

SEQ ID NO: 10 - protein, 299 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNTRGGGSGGGGPWNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - DNA, 903 bp, synthetic construct
  source 1..903: mol_type = other DNA
atgttaggtatctggactctgctgcctctagtgttgacctctgtcgctagactgagcagtaaatcagtcaacgctcaggtgactgatattaactccaaagggctggagcttcggaaaacggtgacaacagttgagacccagaacctggagggacttcaccacgacggtcagttttgccacaaaccttgtccccctggcgaacgaaaagcacgagattgcactgtcaacggagacgagccagactgcgtcccctgtcaggagggcaaagagtacactgataaggcccacttttcctcaaagtgccgccgctgtaggctgtgtgacgaaggccacgggctcgaagttgaaatcaattgcacgagaacacagaacactaagtgtcgttgcaagccgaatttcttttgtaacagcacagtctgcgagcactgcgacccttgtacgaagtgtgagcacgggattattaaggagtgtaccctaacctccaataccaagtgcaaggaagaaggaagcaggagcaactgaacgcgtggcggaggttcaggaggaggtggcccatggaattgggtcaacgttattagtgacctaaaaaagattgaagacctgattcagtctatgcacatcgacgcaaccctctacacagagtcggacgtgcaccctagctgcaaggttacggcaatgaagtgcttccttctcgaacttcaggttatttcacttgaatccggtgacgcttcgattcacgacactgtcgaaaatctgataatactcgccaacaactcccttagctcgaacgggaacgtaaccgagagcgggtgcaaagagtgcgaagagttagaggaaaaaaatattaaggaatttctgcagtcctttgtgcatattgtccaaatgtttataaatactagctga

SEQ ID NO: 12 - protein, 299 aa, synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCSRCRLCDEGHGLEVEINCTRTQNTKCRCKPNFFCNSTVCEHCDPCTKCEHGIIKECTLTSNTKCKEEGSRSNTRGGGSGGGGPWNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 903 bp, synthetic construct
  source 1..903: mol_type = other DNA
atgttaggtatctggactctgctgcctctagtgttgacctctgtcgctagactgagcagtaaatcagtcaacgctcaggtgactgatattaactccaaagggctggagcttcggaaaacggtgacaacagttgagacccagaacctggagggacttcaccacgacggtcagttttgccacaaaccttgtccccctggcgaacgaaaagcacgagattgcactgtcaacggagacgagccagactgcgtcccctgtcaggagggcaaagagtacactgataaggcccacttttcctcaaagtgctctcgctgtaggctgtgtgacgaaggccacgggctcgaagttgaaatcaattgcacgagaacacagaacactaagtgtcgttgcaagccgaatttcttttgtaacagcacagtctgcgagcactgcgacccttgtacgaagtgtgagcacgggattattaaggagtgtaccctaacctccaataccaagtgcaaggaagaaggaagcaggagcaactgaacgcgtggcggaggttcaggaggaggtggcccatggaattgggtcaacgttattagtgacctaaaaaagattgaagacctgattcagtctatgcacatcgacgcaaccctctacacagagtcggacgtgcaccctagctgcaaggttacggcaatgaagtgcttccttctcgaacttcaggttatttcacttgaatccggtgacgcttcgattcacgacactgtcgaaaatctgataatactcgccaacaactcccttagctcgaacgggaacgtaaccgagagcgggtgcaaagagtgcgaagagttagaggaaaaaaatattaaggaatttctgcagtcctttgtgcatattgtccaaatgtttataaatactagctga